(19) United States
(12) Patent Application Publication
(10) Pub. No.: US 2023/0357751 A1
(43) Pub. Date: Nov. 9, 2023

(54) METHODS FOR PURIFICATION OF MESSENGER RNA

(71) Applicant: TRANSLATE BIO, INC., Waltham, MA (US)

(72) Inventors: Jonathan ABYSALH, Bolton, MA (US); Frank DEROSA, Chelmsford, MA (US); Cameron SMITH, Cambridge, MA (US); Jorel E. VARGAS, Derry, NH (US)

(21) Appl. No.: 18/247,607

(22) PCT Filed: Oct. 1, 2021
(86) PCT No.: PCT/US2021/053182

§ 371 Date: Mar. 31, 2023

Related U.S. Application Data
(60) Provisional application No. 63/086,095, filed on Oct. 1, 2020.

(51) Int. Cl. B04B 11/08; B01D 33/46; B01D 61/14; B01D 61/18; C12N 15/10; B04B 3/00; B04B 7/18; B04B 15/06; B01D 39/08; B01D 63/16; B01D 33/11

(52) U.S. Cl. CPC C12N 15/1017; B01D 33/11; B01D 33/463; B01D 39/083; B01D 61/145; B01D 61/147; B01D 61/18; B01D 63/16; B04B 3/00; B04B 7/18; B04B 11/08; B04B 15/06; B01D 2239/1216; B01D 2315/02

(57) ABSTRACT

The present invention relates, in part, to methods, systems and processes for large-scale purification of mRNA using a filtering centrifuge operating at lower gravitational forces. The invention also relates to compositions of purified mRNA and uses thereof.

### RELATED APPLICATIONS

This application claims priority to U.S. Provisional Application Serial No. 63/086,095, filed Oct. 1, 2020, the disclosures of which are hereby incorporated by reference.

### BACKGROUND OF THE INVENTION

Messenger RNA (mRNA) therapeutics are promising new therapeutic agents; for example, mRNA replacement therapeutics can be alternatives to traditional protein replacement therapies. In an mRNA replacement therapeutic, an intact mRNA encoding a specific protein sequence is delivered to a target cell and is translated into an intact protein by the cell’s native translational machinery. mRNA for such therapeutics typically are synthesized using in vitro transcription systems with enzymes such as RNA polymerases transcribing mRNA from a template such as plasmid DNA, along with or followed by addition of a 5′-cap and 3′-polyadenylation. The result of such reactions is a composition which includes full-length mRNA and various undesirable contaminants, e.g., proteins, salts, buffers, and non-RNA nucleic acids, which are typically omitted to provide a clean and homogeneous mRNA that is usable in an mRNA replacement therapeutic.

Traditionally, mRNA is purified from in vitro transcription reactions by either commercially-available silica-based column systems, such as the Qiagen RNeasy® kit, or by protein extraction into an organic mix (phenol:chloroform:isoamyl alcohol) and subsequent ethanol precipitation. These methods are limited in scale as they can provide maximally five to ten mg of clean and homogeneous mRNA; thus, they are inadequate for the needs of clinical and commercial uses of mRNA. Recent novel methods, such as tangential flow filtration (TFF), have been modified to purify precipitated mRNA from in vitro transcription reactions; this has greatly increased the scale of purification. Additional methods suitable for the large-scale purification of mRNA can be useful for the clinical and commercial development of mRNA therapeutics. For example, another method uses filtration centrifugation. However, many of these methods require large volumes of wash buffer in the purification process to achieve wash efficiency suitable for clinical preparations. These large volumes of wash buffer, often comprising ethanol, restrict batch size in light of safety regulations, which limit the amount of flammable solvent that can be stored in a facility. Accordingly, these known methods can often be employed only with smaller batch sizes, without reconfiguring existing facilities.

Accordingly, a need exists for a cost-effective manner and scalable method that avoids the disadvantages of the prior art processes and produces clean and homogeneous mRNA compositions with a level of purity and integrity that is acceptable for therapeutic uses.

### SUMMARY OF THE INVENTION

The present invention provides, among other things, a highly efficient and cost-effective method of purifying messenger RNA (mRNA). The method involves precipitating an impure RNA preparation and purifying it using a filtering centrifuge. The present invention is, in part, based on the surprising discovery that loading a suspension comprising precipitated mRNA into a filtering centrifuge and washing the retained precipitated mRNA can be done at lower centrifuge speed to those used previously. In particular, the loading step can be performed at a lower centrifuge speed while still ensuring that the mRNA can be effectively washed and purified. This is counterintuitive because higher centrifuge speeds are used in the art for loading a filtering centrifuge. It was thought that the higher speeds are necessary to ensure the precipitated mRNA in the suspension is effectively retained by the filter avoiding the resulting cake from being dislodged. Surprisingly, the inventors found that the use of lower centrifuge speeds at both the loading and washing steps reduces the volume of volatile organic solvent (e.g., ethanol) required during the purification process. Indeed, in some aspects of the invention, the use of volatile organic solvent (e.g., ethanol) can be avoided completely, while using a lower speed for loading and washing the precipitated mRNA. In line with these observations, the methods of the invention can use the same lower centrifuge speed for both the loading and washing steps, streamlining and automating the purification process, both lending themselves to increased scalability compared to previous methods. Therefore, the present invention provides an effective, reliable, and safer method of purifying mRNA, which can be adapted for large-scale manufacturing processes using existing manufacturing facilities, providing a very high yield of mRNA with clinical grade integrity and purity.

In one aspect, the present invention provides a method for purifying messenger RNA (mRNA), the method comprising the steps of a) precipitating mRNA from a solution comprising one or more protein and/or short abortive transcript contaminants from manufacturing the mRNA to provide a suspension comprising precipitated mRNA; b) loading the suspension comprising the precipitated mRNA into a filtering centrifuge comprising a filter wherein the precipitated mRNA is retained by the filter; c) washing the retained precipitated mRNA by adding a wash buffer to the filtering centrifuge; and d) recovering the retained precipitated mRNA from the filter, wherein the filtering centrifuge is operated during loading step (b) and washing step (c) at a centrifuge speed that exerts a gravitational (g) force of less than 1300 g.

In some embodiments, the centrifuge speed exerts a gravitational (g) force of between about 150 g and about 1300 g. In some embodiments, the centrifuge speed exerts a gravitational (g) force of between about 300 g and about 1300 g, for example, between about 400 g and about 1100 g. In some embodiments, the centrifuge speed exerts a gravitational (g) force of between about 500 g and about 900 g, for example, between about 550 g and about 850 g. In some embodiments, the centrifuge speed exerts a gravitational (g) force of between about 550 g and about 750 g. In some embodiments, the centrifuge speed exerts a gravitational (g) force of between about 650 g and about 750 g. In particular embodiments, the centrifuge speed exerts a gravitational (g) force of between about 700 g and about 900 g, for example between about 750 g and 850 g (e.g. about 800 g).

In some embodiments, the filtering centrifuge is operated at the same centrifuge speed during loading step (b) and washing step (c).

In some embodiments, the recovering the retained precipitated mRNA from the filter comprises the steps of (i) solubilising the retained precipitated mRNA; and (ii) collecting the solubilised mRNA.

In some embodiments, precipitating the mRNA comprises adding one or more agents that promote precipitation of mRNA, for example one or more of an alcohol, an amphiphilic polymer, a buffer, a salt, and/or a surfactant. In some embodiments, the one or more agents that promote precipitation of the mRNA are: a salt, and an alcohol or an amphiphilic polymer. In some embodiments, the alcohol is ethanol. In some embodiments, the salt is a chaotropic salt. In some embodiments, the salt is at a final concentration of 2-4 M, for example of 2.5-3 M. In particular embodiments, the salt is at a final concentration of about 2.7 M. Guanidinium thiocyanate (GSCN) is a chaotropic salt particularly suitable for the method of the present invention. In some embodiments, the amphiphilic polymer is selected from pluronics, polyvinyl pyrrolidone, polyvinyl alcohol, polyethylene glycol (PEG), triethylene glycol monomethyl ether (MTEG), or combinations thereof.

In some embodiments, the molecular weight of PEG is about 200 to about 40,000 g/mol. In some embodiments, the molecular weight of PEG is about 200-600 g/mol, about 2000-10000 g/mol, or about 4000-8000 g/mol. In particular embodiments, the molecular weight of PEG is about 6000 g/mol (for example, PEG-6000).

In some embodiments, the PEG is at a final concentration of about 10% to about 100% weight/volume. In some embodiments, the PEG is at a final concentration of about 50% weight/volume. In some embodiments, the PEG is at a final concentration of less than 25% weight/volume. In some embodiments, the PEG is at a final concentration of about 5% to 20% weight/volume. In particular embodiments, the PEG is at a final concentration of about 10% to 15% weight/volume.

In some embodiments, the amphiphilic polymer is MTEG. In some embodiments, the MTEG is at a final concentration of about 10% to about 100% weight/volume concentration. In some embodiments, the MTEG is at a final concentration of about 15% to about 45% weight/volume, for example of about 20% to about 40% weight/volume. In some embodiments, the MTEG is at a final concentration of about 20%, about 25%, about 30%, or about 35% weight/volume. In particular embodiments, the MTEG is at a final concentration of about 25% weight/volume.

In some embodiments, the suspension comprises precipitated mRNA, a salt and MTEG. In some embodiments, the salt in the suspension is guanidinium thiocyanate (GSCN). In some embodiments, the suspension is free of alcohol, for example ethanol.

In some embodiments, step (a) of the method of the invention further comprises adding at least one filtration aid to the suspension comprising precipitated mRNA. In some embodiments, the precipitated mRNA and the at least one filtration aid are at a mass ratio of about 1:2; about 1:5; about 1:10 or about 1:15. In particular embodiments, the precipitated mRNA and the at least one filtration aid are at a mass ratio of about 1:10. In some embodiments, the filtration aid is a dispersant. In some embodiments, the dispersant is one or more of ash, clay, diatomaceous earth, glass beads, plastic beads, polymers, polymer beads (e.g., polypropylene beads, polystyrene beads), salts (e.g., cellulose salts), sand, and sugars. In particular embodiments, the polymer is a naturally occurring polymer, e.g. cellulose (for example, powdered cellulose fibre).

In some embodiments, the suspension comprises at least 100 mg, 1 g, 10 g, 100 g, 250 g, 500 g, 1 kg, 10 kg, 100 kg, one metric ton, or ten metric tons, of mRNA or any amount there between. In some embodiments, the suspension comprises greater than 1 kg of mRNA.

In some embodiments, the filter comprises a porous substrate. In some embodiments, the porous substrate is a filter cloth, a filter paper, a screen and a wire mesh. In some embodiments, the filter is a microfiltration membrane or ultrafiltration membrane. In some embodiments, the filter has an average pore size is about 0.5 micron or greater, about 0.75 micron or greater, about 1 micron or greater, about 2 microns or greater, about 3 microns or greater, about 4 microns or greater, or about 5 microns or greater. In some embodiments, [0017] the filter has an average pore size of about 0.01 micron to about 200 microns, about 1 micron to about 2000 microns, about 0.2 microns to about 5 micron, or about one micron to about 3 microns, e.g. about 1 micron. In particular embodiments, the filter cloth is a polypropylene cloth having an average pore size of about 1 micron.

In some embodiments, the volume of wash buffer for washing the retained precipitated mRNA is between about 0.5 L/g mRNA and about 8 L/g mRNA. In some embodiments, the volume of wash buffer for washing the retained precipitated mRNA is less than 2 L/g mRNA. In some embodiments, the volume of wash buffer for washing the retained precipitated mRNA is between about 0.5 L/g mRNA and about 1.5 L/g mRNA, e.g., about 0.5 L/g mRNA. In particular embodiments, the volume of wash buffer for washing the retained precipitated mRNA is about 0.5 L/g mRNA or less.

In some embodiments, the wash buffer is loaded into the filtering centrifuge at a rate of about 1 liter/min to about 60 liter/min, e.g., at a rate of about 5 liter/min to about 45 liter/min. In some embodiments, the total volume of wash buffer is loaded into the filtering centrifuge in between about 0.5 hours to about 4 hours, for example by using filtering centrifuges having a rotor size (i.e. basket diameter) of about 30 cm to about 170 cm. In some embodiments, the retained precipitated mRNA is washed to a purity of between about 50% to about 100% in between about 0.5 hours to about 4 hours, for example less than about 90 minutes. In particular embodiments, the retained precipitated mRNA is washed to a purity of at least 95% in less than 90 minutes. In some embodiments, the wash buffer is loaded into the filtering centrifuge at a rate that depends on the surface area (i.e. m²) of the filter of the filtering centrifuge (e.g. about 5 liter/min/m² to about 25 liter/min/m², for example about 15 liter/min/m²).

In some embodiments, the wash buffer comprises one or more of an alcohol, an amphiphilic polymer, a buffer, a salt, and/or a surfactant. In some embodiments, the wash buffer comprises an alcohol or an amphiphilic polymer.

In some embodiments, the wash buffer comprises ethanol. In some embodiments, the ethanol is at about 80% weight/volume concentration.

In some embodiments, the wash buffer comprises an amphiphilic polymer selected from pluronics, polyvinyl pyrrolidone, polyvinyl alcohol, polyethylene glycol (PEG), triethylene glycol monomethyl ether (MTEG), or combinations thereof.

In some embodiments, the amphiphilic polymer is PEG. In some embodiments, the PEG is present in the wash solution at about 10% to about 100% weight/volume concentration. In some embodiments, the PEG is present in the wash solution at about 50% to about 95% weight/volume concentration. In particular embodiments, the PEG is present in the wash solution at about 90% weight/volume concentration. In some embodiments, the molecular weight of the PEG is about 100 to about 1,000 g/mol. In some embodiments, the molecular weight of PEG is about 200-600 g/mol. In some embodiments, the molecular weight of PEG is about 400 g/mol (for example PEG-400).

In some embodiments, wherein the amphiphilic polymer is MTEG. In some embodiments, the MTEG is present in the wash solution at about 75%, about 80%, about 85%, about 90% or about 95% weight/volume concentration. In some embodiments, the MTEG is present in the wash solution at about 90% weight/volume concentration or about 95% weight/volume concentration. In particular embodiments, the MTEG is present in the wash solution at about 95% weight/volume concentration.

In some embodiments, the wash buffer is free of alcohol, for example ethanol.

In some embodiments, the recovering the retained mRNA occurs while the filtering centrifuge is in operation. In some embodiments, the recovering the retained mRNA occurs via a blade that removes the retained precipitated mRNA from the filter of the filtering centrifuge. In some embodiments, the recovering the retained mRNA occurs while the filtering centrifuge is not in operation.

In some embodiments, the purification method according to the invention is free of alcohol, for example ethanol.

In some embodiments, the solubilising the retained mRNA comprises dissolving the mRNA in an aqueous medium. In some embodiments, the aqueous medium comprises water, a buffer (e.g., Tris- EDTA (TE) buffer or sodium citrate buffer), a sugar solution (e.g., a sucrose or trehalose solution), or combinations thereof. In some embodiments, the aqueous medium is water for injection. In some embodiments, the aqueous medium is TE buffer. In some embodiments, the aqueous medium is a 10% trehalose solution. In some embodiments, the solubilising occurs within the filtering centrifuge. In some embodiments, the solubilising occurs outside the filtering centrifuge.

In some embodiments, the collecting of the solubilised mRNA comprises one or more steps of separating the filtration aid from the solubilised mRNA. In some embodiments, the one or more steps for separating the filtration aid from the solubilised mRNA comprise applying the solution comprising the solubilised mRNA and filtration aid to a filter, wherein the filtration aid is retained by the filter, yielding a solution of purified mRNA. In particular embodiments, the suspension comprising the solubilised mRNA and filtration aid is applied to a filter of a filtering centrifuge by centrifugation. In some embodiments, the centrifugation is at a gravitational (g) force of less than 3100 g, e.g., between about 1000 g and about 3000 g.

In some embodiments, the filtering centrifuge is a continuous centrifuge and/or the filtering centrifuge is orientated vertically or horizontally or the centrifuge is an inverted horizontal centrifuge. In some embodiments, the filtering centrifuge comprises a sample feed port and/or a sample discharge port.

In some embodiments, the mRNA suspension is loaded into the filtering centrifuge at a rate of about 1 liter/min to about 60 liter/min, e.g., at a rate of about 5 liter/min to about 45 liter/min. In some embodiments, the total mRNA suspension is loaded into the filtering centrifuge in between about 0.5 hours to about 8 hours, for example by using filtering centrifuges having a rotor size (i.e. basket diameter) of about 30 cm to about 170 cm.

In some embodiments, the manufacturing the mRNA comprises in vitro transcription (IVT) synthesis of the mRNA. In some embodiments, manufacturing the mRNA comprises a separate step of 3′-tailing of the mRNA. In some embodiments, the separate step of 3′-tailing of the mRNA further comprising 5′ capping of the mRNA. In some embodiments, IVT synthesis of the mRNA comprises 5′-capping and optionally 3′-tailing of the mRNA.

In particular embodiments, the steps (a) through (d) of the method of the present invention are performed after IVT synthesis of the mRNA. In some embodiments, the volume of wash buffer for washing the retained precipitated mRNA after IVT synthesis is less than 8 L/g mRNA, e.g., less than 6 L/g mRNA or less than 5 L/g mRNA. In some embodiments, the volume of wash buffer for washing the retained precipitated mRNA after IVT synthesis is between about 0.5 L/g mRNA and about 4 L/g mRNA. In some embodiments, the volume of wash buffer for washing the retained precipitated mRNA after IVT synthesis is between about 0.5 L/g mRNA and about 1.5 L/g mRNA.

In some embodiments, steps (a) through (d) of the present invention are performed after IVT synthesis of the mRNA and again after the separate step of 3′-tailing of the mRNA. In some embodiments, the total volume of wash buffer for washing the retained precipitated mRNA after IVT synthesis and/or after the separate step of 3′-tailing of the mRNA is less than 8 L/g mRNA, e.g., less than 6 L/g mRNA or less than 5 L/g mRNA. In some embodiments, the total volume of wash buffer for washing the retained precipitated mRNA after IVT synthesis and/or after the separate step of 3′-tailing of the mRNA is between about 0.5 L/g mRNA and about 4 L/g mRNA. In some embodiments, the total volume of wash buffer for washing the retained precipitated mRNA after IVT synthesis and/or after the separate step of 3′-tailing of the mRNA is between about 0.5 L/g mRNA and about 1.5 L/g mRNA, for example about 1 L/g mRNA. In particular embodiments, the volume of wash buffer for washing the retained precipitated mRNA after IVT synthesis is about 0.5 L/g mRNA. In a particular embodiment, the volume of wash buffer for washing the retained precipitated mRNA after the separate step of 3′-tailing and/or capping of the mRNA is about 0.5 L/g mRNA. In a specific embodiment, the total volume of wash buffer for washing the retained precipitated mRNA after IVT synthesis and after the separate step of 3′-tailing and/or 5′-capping of the mRNA is about 1 L/g mRNA.

In some embodiments, the mRNA is or greater than about 1 kb, 1.5 kb, 2 kb, 2.5 kb, 3 kb, 3.5 kb, 4 kb, 4.5 kb, 5 kb, 6 kb, 7 kb, 8 kb, 9 kb, 10 kb, 11 kb, 12 kb, 13 kb, 14 kb, 15 kb, or 20 kb in length.

In some embodiments, the mRNA comprises one or more nucleotide modifications. In some embodiments, the one or more nucleotide modifications comprises modified sugars, modified bases, and/or modified sugar phosphate backbones.

In some embodiments, the mRNA is comprises no nucleotide modifications.

In some embodiments, the recovery of purified mRNA is at least 10 g, 20 g, 50 g, 100 g, 250 g, 500 g, 1 kg, 5 kg, 10 kg, 50 kg, or 100 kg per single batch. In one embodiment, the recovery of purified mRNA is at least 250 g per single batch. In another embodiment, the recovery of purified mRNA is at least 500 g per single batch. In a particular embodiment, the recovery of purified mRNA is at least 1 kg per single batch. In some embodiments, the total purified mRNA is recovered in an amount that results in a yield of at least about 80%, 85%, 90%, 95%, 96%, 97%, 98%, 99%, or about 100%. In some embodiments, the total purified mRNA is recovered in an amount that results in a yield of about 80% to about 100%. In some embodiments, the total purified mRNA is recovered in an amount that results in a yield of about 90% to about 99%. In particular embodiments, the total purified mRNA is recovered in an amount that results in a yield of at least about 90%.

In some embodiments, the purity of the purified mRNA is between about 60% and about 100%. In some embodiments, the purity of the purified mRNA is between about 80% and 99%. In some embodiments, the purity of the purified mRNA is between about 90% and about 99%.

In some embodiments, the purified mRNA has an integrity of at least about 80%, 85%, 90%, 92%, 94%, 95%, 96%, 97%, 98%, or 99%. In some embodiments, the purified mRNA has an integrity of or greater than about 95%. In some embodiments, the purified mRNA has an integrity of or greater than about 98%. In particular embodiments, the purified mRNA has an integrity of or greater than about 99%.

In some embodiments, wherein the purified mRNA has a clinical grade purity without further purification. In some embodiments, the clinical grade purity is achieved without the further purification selected from high performance liquid chromatography (HPLC) purification, ligand or binding based purification, tangential flow filtration (TFF) purification, and/or ion exchange chromatography.

In some embodiments, the purified mRNA comprises 5% or less, 4% or less, 3% or less, 2% or less, 1% or less or is substantially free of protein contaminants as determined by capillary electrophoresis. In some embodiments, the purified mRNA comprises less than 5%, less than 4%, less than 3%, less than 2%, less than 1%, or is substantially free of salt contaminants determined by high performance liquid chromatography (HPLC). In some embodiments, the purified mRNA comprises 5% or less, 4% or less, 3% or less, 2% or less, 1% or less or is substantially free of short abortive transcript contaminants determined by high performance liquid chromatography (HPLC). In some embodiments, the purified mRNA has integrity of 95% or greater, 96% or greater, 97% or greater, 98% or greater, or 99% or greater as determined by capillary electrophoresis.

In some embodiments, the one or more protein and/or short abortive transcript contaminants include enzyme reagents use in IVT mRNA synthesis. In particular embodiments, the enzyme reagents include a polymerase enzyme (e.g., T7 RNA polymerase or SP6 RNA polymerase), DNAse I, pyrophosphatase and a capping enzyme.

In some embodiments, the method of the invention also removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method of the invention also removes RNAse inhibitor.

In another aspect, the present invention provides a purified mRNA obtained by any one of the methods of the present invention.

In another aspect, the present invention provides a composition comprising a purified mRNA obtained by any one of the methods of the present invention. In some embodiments, the composition further comprises at least one pharmaceutically acceptable excipient.

In another aspect, the present invention provides a method for treating a disease or disorder comprising administering to a subject in need thereof a purified mRNA or a composition comprising a purified mRNA obtained by any one of the methods of the present invention.

In another aspect, the present invention provides a purified mRNA or a composition comprising a purified mRNA obtained by any one of the methods of the present invention for use in therapy.

In another aspect, the present invention provides a process for purifying mRNA, the process comprising the steps of: I) providing a suspension comprising precipitated mRNA in a first vessel, wherein the precipitated mRNA comprises one or more protein and/or short abortive transcript contaminants from manufacturing the mRNA; II) providing a wash buffer in a second vessel; III) transferring the content of the first vessel into a filtering centrifuge comprising a filter, wherein the transferring occurs at a rate of about 5 liter/min/m² to about 25 liter/min/m² with respect to the surface area of the filter of the filtering centrifuge (e.g. about 15 liter/min/m²) while the filtering centrifuge is in operation at a first centrifuge speed such that the precipitated mRNA is retained on the filter of said filtering centrifuge; IV) transferring the content of the second vessel into the filtering centrifuge, wherein the transferring occurs at a rate of about 5 liter/min/m² to about 25 liter/min/m² with respect to the surface area of the filter of the filtering centrifuge (e.g. about 15 liter/min/m²) while the filtering centrifuge remains in operation at the first centrifuge speed, thereby washing the precipitated mRNA retained on the filter of said filtering centrifuge with the wash buffer; and V) recovering the washed precipitated mRNA from the filter of said filtering centrifuge.

In some embodiments, the first centrifuge speed exerts a gravitational (g) force of less than 1300 g.

In some embodiments of the process of the present invention, the transferring in steps (III) and (IV) is by pumping. In some embodiments, the pumping in steps (III) and (IV) is by a single pump operably linked to the first and second vessels.

In some embodiments of the process of the present invention, one or more valves control the transferring from the first vessel and the second vessel.

In some embodiments of the process of the present invention, the content of the first vessel and the content of the second vessel are transferred to the filtering centrifuge via a sample feed port.

In some embodiments of the process of the present invention, the filter of the filtering centrifuge is rinsed with water for injection comprising 1% 10 N NaOH after step (V).

In some embodiments of the process of the present invention, the suspension comprising precipitated mRNA includes a filtration aid.

In some embodiments of the process of the present invention, the process further comprises: i) solubilising the washed precipitated mRNA comprising the filtration aid, which was recovered in step (V); ii) transferring the solubilised mRNA from step (i) into a or said filtering centrifuge at a rate of about 5 liter/min/m² to about 25 liter/min/m² with respect to the surface area of the filter of the filtering centrifuge (e.g. about 15 liter/min/m²), wherein the filtering centrifuge comprises a filter for retaining the filtration aid; and iii) collecting the solubilised purified mRNA from the filtering centrifuge by centrifugation.

In some embodiments of the process of the present invention, the transferring is done through a sample feed port of the filtering centrifuge.

In some embodiments of the process of the present invention, step (iii) comprises collecting the solubilised purified mRNA via a sample discharge port of the filtering centrifuge.

In a further aspect, the present invention provides a system for purifying mRNA, wherein the system comprises: a) a first vessel for receiving precipitated mRNA; b) a second vessel for receiving wash buffer; c) a third vessel for receiving the washed precipitated mRNA and/or an aqueous medium for solubilising precipitated mRNA; d) a filtering centrifuge comprising:
i) a filter, wherein the filter is arranged and dimensioned to retain precipitated mRNA and/or a filtration aid, and to let pass solubilised mRNA;
ii) a sample feed port; and
iii) a sample discharge port;
e) a fourth vessel for receiving purified mRNA, wherein said vessel is connected to the sample discharge port of the filtering centrifuge; f) a pump configured to direct flow through the system at a rate of about 5 liter/min/m² to about 25 liter/min/m² with respect to the surface area of the filter of the filtering centrifuge (e.g. about 15 liter/min/m²); wherein the first vessel, the second vessel and the third vessel are operably linked to an input of the pump, and wherein the sample feed port of the filtering centrifuge is connected to an output of the pump; and g) one or more valves configured to preclude simultaneous flow from the first, second and third vessels.

In some embodiments of the system of the present invention, the system further comprises a data processing apparatus comprising means for controlling the system to carry out any of the methods of the present invention. In some embodiments, the data processing apparatus is (a) a computer program comprising instructions or (b) a computer-readable storage medium comprising instructions.

In a further aspect, the present invention also provides a composition comprising 10-1000 g mRNA, amphiphilic polymer and a filtration aid at relative concentrations of about 1:1:10 in a sterile, RNase-free container.

In some embodiments of the composition of the present invention, the amphiphilic polymer comprises PEG having a molecular weight of about 2000-10000 g/mol; 4000-8000 g/mol or about 6000 g/mol (for example PEG-6000). In some embodiments, the amphiphilic polymer comprises MTEG. In some embodiments, the filtration aid is cellulose-based.

Various aspects of the invention are described in detail in the following sections. The use of sections is not meant to limit the invention. Each section can apply to any aspect of the invention. In this application, the use of “or” means “and/or” unless stated otherwise.

### BRIEF DESCRIPTION OF THE DRAWING

The following figures are for illustration purposes only and not for limitation.

FIG. 1 is a photograph of a kilogram-scale laboratory filtering centrifuge with a 15 cm basket.

FIG. 2 is a photograph of a kilogram-scale horizontal filtering peeler centrifuge with a 30 cm basket.

FIG. 3 shows the configuration of the components of an exemplary system of the present invention or for use in the method or process of the present invention.

FIG. 4 shows a flow chart outlining exemplary steps of a method or process of the invention. The dashed lines represent optional steps in the process or method.

FIG. 5 shows a schematic diagram outlining the steps of an exemplary process of the present invention using an exemplary system of the present invention.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Approximately or about: As used herein, the term “approximately” or “about,” as applied to one or more values of interest, refers to a value that is similar to a stated reference value. In certain embodiments, the term “approximately” or “about” refers to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein are modified by the term “approximately” or “about”.

Batch: As used herein, the term “batch” refers to a quantity or amount of mRNA purified at one time, e.g., purified according to a single manufacturing order during the same cycle of manufacture. A batch may refer to an amount of mRNA purified in a single purification run.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

dsRNA: As used herein, the term “dsRNA” refers to the production of complementary RNA sequences during an in vitro transcription (IVT) reaction. Complimentary RNA sequences can be produced for a variety of reasons including, for example, short abortive transcripts that can hybridize to complimentary sequences in the nascent RNA strand, short abortive transcripts acting as primers for RNA dependent DNA independent RNA transcription, and possible RNA polymerase template reversal.

Gravitational (g) force: As used herein, the term “gravitational (g) force” refers to the degree of acceleration to be applied to the sample in the centrifuge. Herein, the gravitational (g) force generated by the centrifuge is exerted onto the precipitated mRNA retained on the filter and the other substances which pass through the basket or drum of the filtering centrifuge. The gravitational (g) force generated by a filtering centrifuge is dependent on the size of the centrifuge. As the motion of the basket of a centrifuge is circular, the acceleration force is calculated as the product of the radius and the square of the angular velocity. Historically known as “relative centrifugal force” (RCF), the g force is the measurement of the acceleration applied to the sample within a circular movement and is measured in units of gravity. Herein gravitational (g) force and RCF can be used interchangeably and are not to be confused with revolutions per minute (RPM) of the basket. The gravitational (g) force or RCF is related to RPM according to the radius of the basket and is relative to the force of gravity. The distinction between RPM and RCF is important, as two baskets with different diameters running at the same rotational speed (RPM) will result in different accelerations (with the basket having the larger diameter achieving a higher gravitational (g) force at the same rotational speed).

Converting between gravitational (g) force or RCF and RPM on the basis of different sized centrifugal baskets would be routine to the skilled person. The gravitational (g) force can be determined from the radius of the basket of the filtering centrifuge and the RPM using the following formula:

$$\text{g} = {\left(\text{n}\right)}^{2} \times 1.118 \times {10}^{- 5} \times \text{r}$$

wherein:
g = gravitational (g) force (RCF)
r = rotational radius (cm)
n = revolutions per minute (RPM)

The RPM can be determined from the radius of the basket of the filtering centrifuge and the gravitational (g) force using the following formula:

$$\text{n} = √ \left[\text{g} / \left(\text{r} \times 1.118\right)\right] \times 1 \times {10}^{5}$$

wherein:
g = gravitational (g) force (RCF)
r = rotational radius (cm)
n = revolutions per minute (RPM)
In line with the above, specific filtering centrifuges will have different conversions of RPM to gravitational (g) force and vice versa. Herein, for a centrifuge having a basket diameter of 30 cm (e.g. Heinkel H300P) may exert a gravitational (g) force of about 1996 g at a speed of 3450 RPM. Accordingly, the conversion of RPM to gravitational (g) force is a factor of about 0.578 and the conversion from gravitational (g) force to RPM is a factor of about 1.73. Herein, for a centrifuge having a basket diameter of 50 cm (e.g. Rousselet Robatel EHBL 503) may exert a gravitational (g) force of about 1890 g at a speed of about 2600 RPM. Accordingly, the conversion of RPM to gravitational (g) force is a factor of about 0.723 and the conversion from gravitational (g) force to RPM is a factor of about 1.38.

Impurities: As used herein, the term “impurities” refers to substances inside a confined amount of liquid, gas, or solid, which differ from the chemical composition of the target material or compound. Impurities are also referred to as “contaminants.”

In Vitro: As used herein, the term “in vitro” refers to events that occur in an artificial environment, e.g., in a test tube or reaction vessel, in cell culture, etc., rather than within a multicellular organism.

In Vivo: As used herein, the term “in vivo” refers to events that occur within a multicellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions.

mRNA integrity: As used herein, the term “mRNA integrity” generally refers to the quality of mRNA. In some embodiments, mRNA integrity refers to the percentage of mRNA that is not degraded after a purification process.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

Precipitation: As used herein, the term “precipitation” (or any grammatical equivalent thereof) refers to the formation of a solid in a solution. When used in connection with mRNA, the term “precipitation” refers to the formation of insoluble or solid form of mRNA in a liquid.

Prematurely aborted RNA sequences: The terms “prematurely aborted RNA sequences”, “short abortive RNA species”, “shortmers”, and “long abortive RNA species” as used herein, refers to incomplete products of an mRNA synthesis reaction (e.g., an in vitro synthesis reaction). For a variety of reasons, RNA polymerases do not always complete transcription of a DNA template; e.g., RNA synthesis terminates prematurely. Possible causes of premature termination of RNA synthesis include quality of the DNA template, polymerase terminator sequences for a particular polymerase present in the template, degraded buffers, temperature, depletion of ribonucleotides, and mRNA secondary structures. Prematurely aborted RNA sequences may be any length that is less than the intended length of the desired transcriptional product. For example, prematurely aborted mRNA sequences may be less than 1000 bases, less than 500 bases, less than 100 bases, less than 50 bases, less than 40 bases, less than 30 bases, less than 20 bases, less than 15 bases, less than 10 bases or fewer.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially free: As used herein, the term “substantially free” refers to a state in which relatively little or no amount of a substance to be removed (e.g., prematurely aborted RNA sequences) are present. For example, “substantially free of prematurely aborted RNA sequences” means the prematurely aborted RNA sequences are present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) of the impurity. Alternatively, “substantially free of prematurely aborted RNA sequences” means the prematurely aborted RNA sequences are present at a level less than about 100 ng, 90 ng, 80 ng, 70 ng, 60 ng, 50 ng, 40 ng, 30 ng, 20 ng, 10 ng, 1 ng, 500 pg, 100 pg, 50 pg, 10 pg, or less.

Unless otherwise defined, all technical and scientific terms used herein have the same meaning as commonly understood by one of ordinary skill in the art to which this application belongs and as commonly used in the art to which this application belongs; such art is incorporated by reference in its entirety. In the case of conflict, the present specification, including definitions, will control.

### DETAILED DESCRIPTION OF THE INVENTION

The present invention provides, among other things, improved methods for purifying mRNA using filtration by centrifugation. In addition, the present invention provides compositions produced by the methods of the invention, and processes and systems for carrying out the methods of the invention.

To meet manufacturing demands, a method for mRNA purification needs to be robust and scalable to ensure large-scale manufacturing capabilities are in place to meet all clinical and commercial needs, while providing an equivalent or better product when compared to currently-available industry-standard mRNA purification methods. The inventors have demonstrated that centrifugation filtration can achieve greater than 95% recovery of in vitro synthesized mRNA from process associated contaminants such enzymes and short abortive RNA species, while requiring reduced volumes of wash buffer, by using a lower centrifugation speed both for loading and washing the precipitated mRNA obtained from an in vitro synthesis process compared to currently available methods. In addition, the inventors show that the same process parameters can be used to purify mRNA whether the process for precipitating and washing the in vitro synthesized mRNA employs an organic solvent (e.g., ethanol) or an amphiphilic polymer (e.g., MTEG). Reducing or avoiding the need for organic solvents is highly advantageous. For example, safety restrictions associated with increased volumes of volatile and/or flammable wash buffers limit the scalability of organic solvent-based processes in existing facilities. Using the method of the invention, the inventors demonstrate that a 4-fold smaller volume of washing buffer (1 L/g purified mRNA vs. 4 L/g purified mRNA in prior art methods) can be used to purify mRNA that is manufactured by separately synthesizing the mRNA in a first reaction and then capping and tailing it in a second reaction. Reducing the volumes of wash buffer makes purification more efficient and less costly and reduces the environmental impact.

The reduced centrifuge speed for loading and washing precipitated mRNA obtained from an in vitro synthesis process exerts a gravitational (g) force of less than 1300 g. For example, the inventors have found that centrifuge speeds exerting a gravitational (g) force of less than 1300 g result in a less compact filter cake of purified mRNA that peels more easily and completely and is more readily re-solubilized, further increasing the efficiency and speed of purification. Furthermore, the use of lower speeds during the loading step allow the process to use the same lower centrifuge speed at both the loading and washing steps, providing a more straightforward process, lending itself to automation and increased scalability.

### Centrifugation

Centrifugation has been used in the art for solid-liquid separation. Centrifuges magnify the force of gravity to separate phases (e.g. solids from liquids). Filtering centrifuges exploit a medium, such as a fabric cloth, to retain the solid phase while allowing the liquid phase to pass through. Filtering centrifugation has also been used for mRNA purification.

For example, WO 2018/157141 uses centrifugation through a porous substrate to remove contaminants from a suspension of mRNA. These methods of mRNA purification recommend the use of high centrifuge speeds at the loading step. Indeed, WO 2018/157141 uses centrifuge speeds exerting a gravitational (g) force of between about 1700 g and 2100 g. These higher speeds were thought to be important to ensure that the suspension of precipitated mRNA is effectively retained by the filter of the filtering centrifuge and to avoid the cake of retained precipitated mRNA from being dislodged during the purification process.

As outlined above, the present inventors have demonstrated that high speeds are not necessary at the loading step. In fact, using a lower centrifuge speed to exert a reduced gravitational (g) force at the loading step results in a less dense cake of retained precipitated mRNA. As outlined below, a less dense cake allows, inter alia, for efficient purification of the mRNA using lower volumes of wash buffer and enabling complete removal of the cake from the filter of the filtering centrifuge.

### Filtering Centrifuge

A filtering centrifuge works on the principle of centrifugal force, which is created when a device, usually called a basket or drum, is rotated at high speeds on a fixed axis. A filtering centrifuge is capable of separating solids (e.g., precipitated mRNA) and liquid (e.g., a buffer used in the synthesis of the mRNA) from a solid-liquid mixture by passing the liquid through a filter or screen (e.g., a wire mesh). Such centrifuges may include a removable basket or fixed drum which is perforated to allow fluid flow. The perforated basket or drum may be adapted to accept a porous substrate such as a filter cloth or a filter paper. Typically, the porous substrate is removable. In commonly used filtering centrifuges, a suspension flows from the inside of the centrifuge to the outside, thereby passing through the porous substrate (e.g., a removable porous substrate) and then through the basket or perforated drum. In this way, solid material in a solid-liquid mixture added to the inside of the centrifuge is retained and liquids are removed from the suspension.

Centrifuges suitable for use in the methods of the present invention are well-known in the art. See, e.g., Scott, K. and Hughes, R., “Industrial Membrane Separation Technology”. Springer Science & Business Media, 1996; Tarleton, S. and Wakeman, R., “Filtration: Equipment Selection, Modelling and Process Simulation”, Elsevier, 1999; Tarleton, S. and Wakeman, R., “Solid/Liquid Separation: Scale-up of Industrial Equipment”. Elsevier, 2005; Wakeman, R. and Tarleton, S., “Solid/ Liquid Separation: Principles of Industrial Filtration”. Elsevier, 2005; Tarleton, S. and Wakeman, R., “Solid/liquid separation: equipment selection and process design”. Elsevier, 2006; and Sutherland, K. and Chase, G., “Filters and Filtration Handbook”. Elsevier, 2011, each of which is incorporated herein by reference in their entireties. Also, see US1292758A; US1478660A; US3269028A; US3411631A; US3419148A; US3438500A; US3483991A; US3491888A; US3623613A; US3684099A; US3774769A; US3980563A; US4193874A; US4193874A; US4193874A; US4269711A; US4381236A; US4944874A; US5004540A; US5091084A; US5092995A; US5244567A; US5277804A; US5286378A; US5306423A; US5378364A; US5380434A; US5397471A; US5421997A; US5433849A; US5468389A; US5472602A; US5713826A; US6736968B2; US6736968B2; US6736968B2; US7168571B2; US7425264B2; US8021289B2; US8257587B2; US9126233B2; US9297581B2; US20040108281A1; US20040108281A1; US20050245381A1; US20060021931A1; US20060175245A1; US20080149558A1; US20100120598A1; US20100216623A1; US20120285868A1; US20140360039A1; AU2007350788A1; AU2007350788B2; EP1372862A1; EP3040127A1; EP845296A1; WO2004033105A1; WO2008122067A1; WO2014043541A1; WO2016025862A1; WO2016112426A1; WO2016112427A1; and WO2016112428A1, each of which is incorporated herein by reference in their entireties.

Non-limiting examples of suitable centrifuge types include batch filtering centrifuges, inverting filter centrifuges, pusher centrifuges, peeler centrifuges (e.g., horizontal peeler centrifuge, vertical peeler centrifuge, and siphon peeler centrifuge), pendulum centrifuges, screen/scroll centrifuges, and sliding discharge centrifuges.

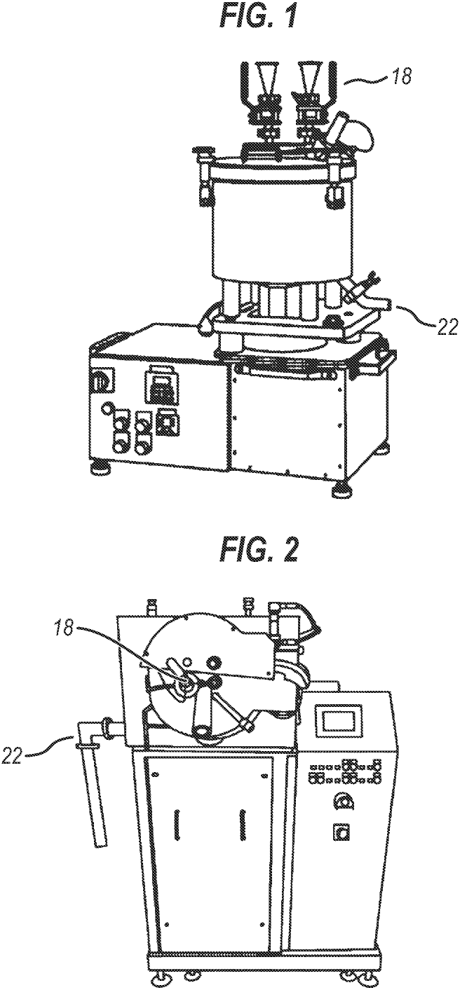

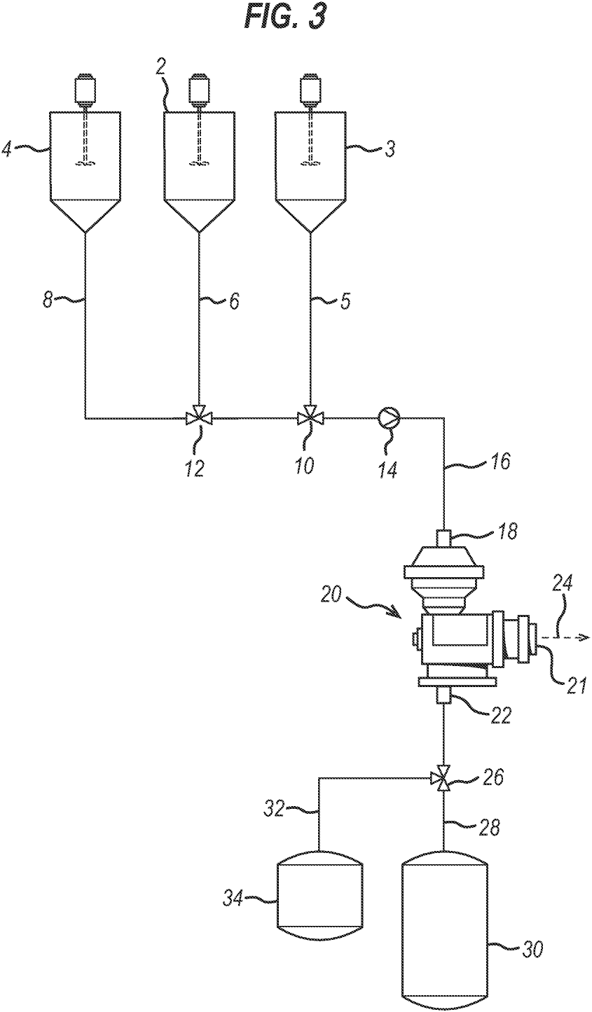

In some embodiments, the filtering centrifuge is a continuous centrifuge. In some embodiments, the filtering centrifuge is orientated vertically. In some embodiments, in some embodiments, the filtering centrifuge is orientated horizontally. In some embodiments, the filtering centrifuge is an inverted horizontal centrifuge. Examples of appropriate filtering centrifuges for use in the methods of the present invention are shown in FIGS. 1 and 2.

In some embodiments, the filtering centrifuge has a basket diameter of about 30 cm to about 170 cm. In a particular embodiment, the filtering centrifuge has a basket diameter of 100 cm or more, for example up to about 170 cm. In some embodiments, the filtering centrifuge has a basket depth of about 15 cm to about 80 cm. In a particular embodiment, the filtering centrifuge has a basket depth of 60 cm or more, for example up to about 80 cm. In some embodiments the filtering centrifuge has a basket diameter:depth of about 30 cm:15 cm to about 170 cm:80 cm. In some embodiments, the filtering centrifuge has a basket diameter of 30 cm and depth of 15 cm. In some embodiments, the filtering centrifuge has a basket diameter of 50 cm and depth of 25 cm. In some embodiments, the filtering centrifuge has a basket diameter of 63 cm and depth of 31.5 cm. In some embodiments, the filtering centrifuge has a basket diameter of 81 cm and depth of 35 cm. In some embodiments, the filtering centrifuge has a basket diameter of 105 cm and depth of 61 cm. In some embodiments, the filtering centrifuge has a basket diameter of 115 cm and depth of 61 cm. In some embodiments, the filtering centrifuge has a basket diameter of 132 cm and depth of 72 cm. In some embodiments, the filtering centrifuge has a basket diameter of 166 cm and a depth of 76 cm. In some embodiments, the filtering centrifuge has a useful volume of about 20 litres to about 725 litres. In some embodiments, the filtering centrifuge has a max load of about 30 kg to about 900 kg. In a particular embodiment, the filtering centrifuge has a max load of more than 250 kg, for example up to 900 kg. In some embodiments, the filtering centrifuge has a maximum filtration surface area of about 0.5 m² to about 4 m². In some embodiments, the filtering centrifuge has a maximum speed (RPM) of 1000 RPM to about 3500 RPM. In some embodiments, the filtering centrifuge can exert a maximum gravitational (g) force of about 900 g to about 2000 g.

### Configuration of the Filtering Centrifuge

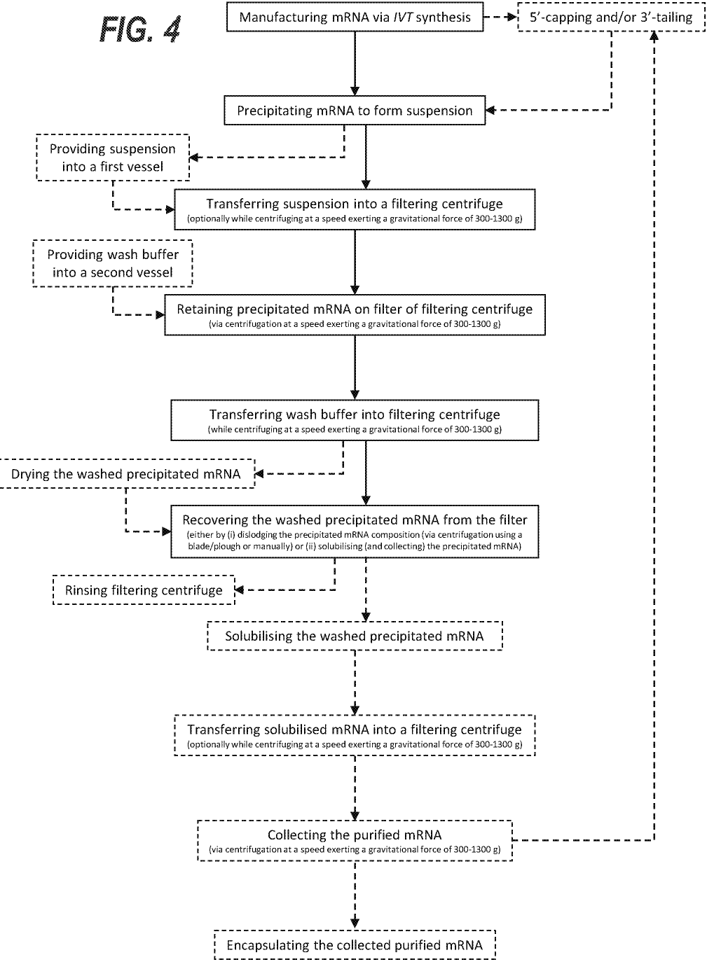

FIG. 3 shows a configuration of a system of the present invention and for use in the methods and processes of the present invention. The system comprises: a first vessel (4) for receiving precipitated mRNA; a second vessel (2) for receiving wash buffer; a third vessel (3) for receiving the washed precipitated mRNA and/or an aqueous medium for solubilising precipitated mRNA; a filtering centrifuge (20) comprising a filter, a sample feed port (18) and a sample discharge port (22); a fourth vessel (34) for receiving purified mRNA and a fifth vessel (30) for receiving contaminants; a pump (14) configured to direct flow through the system; and one or more valves (10, 12 and 26) configured to block simultaneous flow from or to different vessels in the system. The first, second and third vessel are operably linked (5, 6 and 8) to an input of the pump (14) and the sample feed port (18) of the filtering centrifuge is operably linked (16) to an output of the pump (14). The fourth and fifth vessels are operably linked (28 and 34) to the sample discharge port (22) of the filtering centrifuge. Furthermore, the centrifuge comprises a sample discharge channel (21), through which a precipitated mRNA composition can be recovered (21) from the filtering centrifuge. The system displayed in FIG. 3 can be used in methods of the invention comprising either the recovery of the retained washed precipitated mRNA by dislodging a composition of precipitated mRNA from the filter or the recovery of the retained washed precipitated mRNA by solubilisation of the precipitated mRNA retained on the filter and subsequent collection thereof. In a particular embodiment of the invention, the third (3) and fourth vessel (34) are optional components (i.e. the precipitated mRNA can be recovered (24) via the sample discharge channel (21), without requiring a solubilisation step). In another particular embodiment, the third (3) and fourth vessel (34) are used for those embodiments comprising solubilisation of the precipitated mRNA and recovery of purified mRNA (i.e. into the fourth vessel (34)).

In some embodiments, the filtering centrifuge comprises a sample feed port. In some embodiments, the sample feed port receives substances (e.g. a suspension of precipitated mRNA, wash buffer and/or solubilisation buffer) from one or more vessels. In some embodiments, the sample feed port is operably linked to the one or more vessels. In some embodiments, the transfer of the substances from the one or more vessels to the sample feed port of the filtering centrifuge is by pumping. In some embodiments, the pumping is by a single pump operably linked to the one or more vessels and the sample feed port. In some embodiments, the transfer of the substances from the one or more vessels to the sample feed port is controlled by one or more valves.

In some embodiments, the filtering centrifuge comprises a sample discharge port. In some embodiments, the sample discharge port allows recovery of the purified mRNA from the filtering centrifuge. In some embodiments, the sample discharge port is operably linked to one or more vessels for recovering filtered purified mRNA. In some embodiments, the purified mRNA is recovered into one or more vessels for recovering filtered purified mRNA. In some embodiments, the sample discharge port is operably linked to one or more vessels for recovering contaminants during the purification process, for example a waste drum. In some embodiments, the transfer of purified mRNA and/or contaminants from the filtering centrifuge to the one or more vessels via the filter discharge port is by pumping. In some embodiments, the pumping is by a single pump operably linked to the sample discharge port and the one or more vessels for recovering purified mRNA and/or contaminants. In some embodiments, the transfer of purified mRNA and/or contaminants from the filtering centrifuge to the one or more vessels via the filter discharge port is controlled by one or more valves.

In some embodiments, the filtering centrifuge comprises a sample discharge channel configured to receive precipitated mRNA from the basket or drum of the centrifuge upon deploy of the plough or blade of the filtering centrifuge.

### Operating the Filtering Centrifuge

### Loading and Unloading the Filtering Centrifuge

In some embodiments, a pump operably linked to one or more vessels and a sample feed port of a filtering centrifuge is configured to transfer substances from the one or more vessels for providing the suspension of precipitated mRNA, wash buffer and/or solubilisation buffer to the sample feed port at a rate determined as a function of the surface area of the filter of the filtering centrifuge. In some embodiments, the pump is configured to transfer substances from the sample discharge port to the one or more vessels for recovering the purified mRNA and/or contaminants at a rate of about 5 liter/min/m² to about 25 liter/min/m² (with respect to the surface area of the filter of the filtering centrifuge). In some embodiments, the pump is configured to transfer substances from the sample discharge port to the one or more vessels for recovering the purified mRNA and/or contaminants at a rate of about 10 liter/min/m² to about 20 liter/min/m². In some embodiments, the rate of transfer is about 5, 6, 7, 8, 9, 10, 11, 12, 13, 14, 15, 16, 17, 18, 19, 20, 21, 22, 23, 24 or 25 liter/min/m². In particular embodiments, the rate of transfer is about 15 liter/min/m² or less.

In some embodiments, the total volume of suspension, wash buffer and/or solubilisation buffer is loaded into a filtering centrifuge in between about 0.5 hours to about 8 hours, for example about 2 hours to about 6 hours. In some embodiments, the total volume is loaded into the filtering centrifuge in about less than about 8 hours, less than about 7 hours, less than about 6 hours, less than about 5 hours, less than about 4 hours, less than about 3 hours, less than about 2 hours, less than about 1 hour, or less than about 0.5 hours. In some embodiments, the time taken to load the total volume of suspension, wash buffer and/or solubilisation buffer into the filtering centrifuge may depend on the rotor size (i.e. basket diameter) of said filtering centrifuge, for example, loading a total volume of suspension of 1000 g of precipitated mRNA into a filtering centrifuge having a rotor size of about 50 cm may take about 3 hours (see Table D). In some embodiments, the total volume of wash buffer is loaded into the filtering centrifuge in between about 0.5 hours to about 4 hours, for example by using filtering centrifuges having a rotor size (i.e. basket diameter) of about 30 cm to about 170 cm. In some embodiments, the total volume of wash buffer is loaded into the filtering centrifuge in less than about 4 hours, less than about 3 hours, less than about 2 hours, less than about 1 hour, or less than about 0.5 hours. For example, the inventors have achieved impurity removal for a batch of 1000 g of mRNA using 500 litres of wash buffer in about 80 minutes (i.e. at a wash buffer loading rate of 6 L/min or 15 L/min/m²) using a filtering centrifuge having a rotor size of about 50 cm (see Table D).

In some embodiments, the total volume of suspension is loaded into the filtering centrifuge in batches or continuously.

In some embodiments, the total volume of purified mRNA and/or contaminants is recovered from a filtering centrifuge in between about 1 minute to about 90 minutes. In some embodiments, the total volume is recovered from the filtering centrifuge in less than about 90 minutes, less than about 80 minutes, less than about 70 minutes, less than about 60 minutes, less than about 50 minutes, less than about 30 minutes, less than about 20 minutes, less than about 10 minutes, less than about 5 minutes, less than about 4 minutes, less than about 3 minutes, less than about 2 minutes or less than about 1 minute.

In some embodiments, a filtering centrifuge comprises a blade peeler or plough configured to remove precipitated mRNA retained on a filter of the filtering centrifuge. In some embodiments, the blade is deployed while the filtering centrifuge is in operation.

### Centrifuge Speed

The present inventors have found that methods of purifying mRNA using centrifugation filtration achieve higher wash efficiency and increased yield of purified mRNA when centrifuge speeds exerting reduced gravitational (g) force are used. This is counterintuitive given that better filtration was expected at higher speeds. Indeed, as outlined above, WO 2018/157141, employing centrifugation for mRNA purification, uses centrifugation speeds achieving a gravitational (g) force of more than 1500 g, for example around 1750-2250 g to exert maximum force on the precipitated sample in order to improve filtering and to retain contact of the cake with the filter of the filtering centrifuge. The inventors demonstrate herein that the use of centrifuge speeds exerting lower gravitational (g) force achieves equivalent or improved purification with vastly increased wash efficiency (i.e., a lower volume of wash buffer is necessary to clear contaminants from precipitated mRNA).

Without wishing to be bound by theory, the inventors believe that the reduced speed (i.e. reduced gravitational (g) force exerted onto the precipitated mRNA) reduces the density of the cake created by the centrifugation of the precipitated mRNA such that the process of washing the cake is more efficient in light of the reduced packing of the cake. Consequently, the methods of the invention require less wash buffer compared to previous methods in order to achieve clinical grade mRNA purification. Accordingly, the methods of the invention reduce the volume of volatile organic solvent (e.g., alcohol) required for washing the precipitated mRNA in those protocols that include a volatile organic solvent (e.g., alcohol) in the wash buffer. The methods of the invention enable a 75% reduction in wash buffer compared to previous methods, thus allowing significant upscaling of the methods of the invention for larger batch sizes suitable for commercial production of purified clinical grade mRNA.

In addition, the speed of the methods of the invention is increased compared to previous methods, not least in light of the requirement for reduced volumes of wash buffer, allowing more efficient production on a commercial scale. Furthermore, as outlined above, the reduced centrifuge speeds result in a less dense cake product, which has fewer aggregations and a more homogenous consistency. This reduced density improves the efficacy by which the cake can be recovered from the filter of the filtering centrifuge, avoiding potential damage of the filter by the centrifuge blade which can be caused if the cake forms a residual heel. Moreover, the reduced density of the cake also increases the suspension efficacy of the cake, improving the amount of purified mRNA that can be achieved upon solubilisation.

Accordingly, in accordance with the methods of the invention, a centrifuge speed is selected that avoids compacting the filter cake and exerts a gravitational (g) force such that precipitated mRNA is retained on the filter of the filtering centrifuge while the buffers and one or more contaminants pass through it. A centrifuge speed is selected that is appropriate for exerting a particular gravitational (g) force for the loading and washing steps of a method of the invention. In some embodiments, the centrifuge speed is also appropriate for exerting a particular gravitational (g) force for the collecting step of a method of the present invention.

In some embodiments, the centrifuge speed exerts a gravitational (g) force of less than 1300 g. In some embodiments, the centrifuge speed exerts a gravitational (g) force of less than 1200 g. In some embodiments, the centrifuge speed exerts a gravitational (g) force of less than 1100 g. In some embodiments, the centrifuge speed exerts a gravitational (g) force of less than 1000 g. In some embodiments, the centrifuge speed exerts a gravitational (g) force of less than 900 g. In some embodiments, the centrifuge speed exerts a gravitational (g) force of less than 800 g. In some embodiments, the centrifuge speed exerts a gravitational (g) force of less than 700 g. In some embodiments, the centrifuge speed exerts a gravitational (g) force of less than 600 g. In some embodiments, the centrifuge speed exerts a gravitational (g) force of less than 500 g. In some embodiments, the centrifuge speed exerts a gravitational (g) force of less than 400 g. In some embodiments, the centrifuge speed exerts a gravitational (g) force of less than 300 g. In particular embodiments, the centrifuge speed exerts a gravitational (g) force of less than 750 g, for example less than 730 g, for example about 725 g. In particular embodiments, the centrifuge speed exerts a gravitational (g) force of less than 600 g, for example less than 585 g, for example about 575 g.

In some embodiments, the centrifuge speed exerts a gravitational (g) force of between about 150 g and about 1300 g. In some embodiments, the centrifuge speed exerts a gravitational (g) force of between about 250 g and about 900 g. In some embodiments, the centrifuge speed exerts a gravitational (g) force of between about 300 g and about 1300 g. In some embodiments, the centrifuge speed exerts a gravitational (g) force of between about 350 g and about 1250 g. In some embodiments, the centrifuge speed exerts a gravitational (g) force of between about 350 g and about 1050 g. In some embodiments, the centrifuge speed exerts a gravitational (g) force of between about 400 g and about 1100 g. In some embodiments, the centrifuge speed exerts a gravitational (g) force of between about 400 g and about 600 g. In some embodiments, the centrifuge speed exerts a gravitational (g) force of between about 450 g and about 1050 g. In some embodiments, the centrifuge speed exerts a gravitational (g) force of between about 500 g and about 1000 g. In some embodiments, the centrifuge speed exerts a gravitational (g) force of between about 500 g and about 900 g. In particular embodiments, the centrifuge speed exerts a gravitational (g) force of between about 700 g and about 900 g, for example between about 750 g and about 850 g (e.g. about 800 g). This g force has been found to be suitable with a range of centrifuges of different sizes.

In some embodiments, the centrifuge speed exerts a gravitational (g) force of between about 500 g and about 750 g. In some embodiments, the centrifuge speed exerts a gravitational (g) force of between about 550 g and about 850 g. In some embodiments, the centrifuge speed exerts a gravitational (g) force of between about 550 g and about 750 g. In particular embodiments, the centrifuge speed exerts a gravitational (g) force of between about 550 g and about 650 g, for example between about 570 g and 580 g, for example about 575 g. In particular embodiments, the centrifuge speed exerts a gravitational (g) force of between about 650 g and about 750 g, for example between about 720 g and about 730 g, for example about 725 g.

As outlined above, the g force can be calculated on the basis of the basket diameter and the revolutions per minute (RPM). Centrifuging at a speed of 1000 RPM on a centrifuge with a basket diameter of 50 cm exerts a gravitational force of about 725 g. Centrifuging at a speed of 1000 RPM on a centrifuge with a basket diameter of 30 cm exerts a gravitational force of about 575 g. In particular embodiments, irrespective of the basket diameter of the filtering centrifuge, a centrifuge speed that exerts a gravitational (g) force of between about 700 g and about 900 g, for example between about 750 g and about 850 g (e.g. about 800 g), has been found to be particularly suitable to achieve impurity removal.

In some embodiments, the filtering centrifuge is operated at the same centrifuge speed throughout the method of the invention. In some embodiments, the filtering centrifuge is operated at the same centrifuge speed during the loading and washing step of the method of the invention. Maintaining the same centrifuge speed throughout the method of the invention increases the ease and reproducibility of the purification methods of the invention.

In some embodiments, the filtering centrifuge is operated at a centrifuge speed of less than 1500 RPM. In some embodiments, the filtering centrifuge is operated at a centrifuge speed of less than 1250 RPM. In particular embodiments, the filtering centrifuge is operated at a centrifuge speed of less than 1000 RPM. In some embodiments the filtering centrifuge is operated at the same centrifuge speed for both the loading and washing steps.

Exerting the same gravitational force as required by the methods of the invention will demonstrate the same advantages of the methods of the present invention irrespective of the filtering centrifuge and/or the RPM required on said filtering centrifuge to achieve those gravitational (g) forces. Accordingly, the methods of the present invention can be used on any known filtering centrifuge in the art provided that the filtering centrifuge can exert the appropriate gravitational (g) force on the precipitated mRNA. Indeed, the larger commercial centrifuges have a maximum speed and thus a maximum gravitation (g) force which they can exert. For example, the Rousselet Robatel EHBL 1323, having a load capacity of 550 kg, can exert a maximum gravitation (g) force of 1130 g. In light of the inventors’ observations disclosed herein, the methods of the present invention can be applied to larger commercial centrifuges enabling effective purification of mRNA on larger scales.

### Porous Substrate

In a typical embodiment, a filtering centrifuge for use in the methods of the invention comprises a porous substrate (e.g. a filter or membrane). The porous substrate retains precipitated mRNA while allowing solubilised RNA (e.g., short abortive RNA species) to pass through. In some embodiments, the porous substrate can be removed from the filtering centrifuge. As used herein, the term “membrane” or “filter” refers to any porous layer or sheet of material. In this application, the term “membrane” is used inter-changeably with “filter”.

The filter used in any of the methods described herein may feature a variety of filter pore sizes and types. For example, a centrifuge filter can have an average pore size of about 0.01 micron to about 200 microns, about 1 micron to about 2000 microns, about 0.2 microns to about 5 micron, or about 1 micron to about 3 microns. In some embodiments, an average pore size is about 0.5 micron or greater, about 0.75 micron or greater, about 1 micron or greater, about 2 microns or greater, about 3 microns or greater, about 4 microns or greater, or about 5 microns or greater.

In some embodiments, the filter has a pore size appropriate for capturing or retaining precipitated mRNA, while letting impurities (including soluble impurities and/or insoluble with size less than the pore size) pass through as permeate. In some embodiments, the filter has a pore size appropriate for capturing impurities (including insoluble impurities with size more than the pore size, for example a filtration aid), while letting solubilised mRNA pass through. In some embodiments, the filter has an average pore size of or greater than about 0.10 µm, 0.20 µm, 0.22 µm, 0.24 µm, 0.26 µm, 0.28 µm, 0.30 µm, 0.40 µm, 0.5 µm, or 1.0 µm. In some embodiments, the filter has an average pore size of about 0.5 µm to about 2.0 µm. In particular embodiments, the filter has an average pore size of about 1 µm.

In some embodiments, appropriate pore size for retaining precipitated mRNA may be determined by the nominal molecular weight limits (NMWL) of the precipitated mRNA, also referred to as the molecular weight cut off (MWCO). Typically, a filter with pore size less than the NMWL or MWCO of the precipitated mRNA is used. In some embodiments, a filter with pore size two to six (e.g., 2, 3, 4, 5, or 6) times below the NMWL or MWCO of the precipitated mRNA is used. In some embodiments, a suitable filter for the present invention may have pore size of or greater than about 100 kilodaltons (kDa), 300 kDa, 500 kDa, 1,000 kDa, 1,500 kDa, 2,000 kDa, 2,500 kDa, 3,000 kDa, 3,500 kDa, 4,000 kDa, 4,500 kDa, 5,000 kDa, 5,500 kDa, 6,000 kDa, 6,500 kDa, 7,000 kDa, 7,500 kDa, 8,000 kDa, 8,500 kDa, 9,000 kDa, 9,500 kDa, or 10,000 kDa. In some embodiments, the filter has a pore size greater than the NMWL and MWCO of the mRNA but less than the NMWL and MWCO of the precipitated mRNA.

A filter for use in the present invention may be made of any material. Exemplary filter materials include, but are not limited to, polyethersulfone (mPES) (not modified), polyethersulfone (mPES) hollow fiber membrane, polyvinylidene fluoride (PVDF), cellulose acetate, nitrocellulose, MCE (mixed cellulose esters), ultra-high MW polyethylene (UPE), polyfluorotetraethylene (PTFE), nylon, polysulfone, polyether sulfone, polyacrilonitrile, polypropylene, polyvinyl chloride, and combination thereof. For example, fabrics made from thermoplastic polymers, in particular partially crystalline and non-polar thermoplastic polymers (e.g., polyolefins such as polypropylene), have been found to be particularly suitable for use with the invention. Such fabrics can be produced with an average pore size of about 0.5 µm to about 2.0 µm. (e.g., an average pore size of about 1.0 µm).

A suitable filter for use in the present invention may have various surface area. In some embodiments, the filter has a sufficiently large surface area to facilitate large scale production of mRNA. For example, the filter may have a surface area of or greater than about 2,000 cm², 2,500 cm², 3,000 cm², 3,500 cm², 4,000 cm², 4,500 cm², 5,000 cm², 7,500 cm², 10,000 cm², 5 m², 10 m², 12 m², 15 m², 20 m², 24 m², 25 m², 30 m², or 50 m².

Methods herein can accommodate a variety of filter pore sizes while still retaining mRNA and without fouling a filter.

### Process Steps

The methods of the invention relate to the purification of in vitro synthesized mRNA through a series of steps that include precipitation of the in vitro synthesized mRNA to yield a suspension comprising precipitated mRNA, loading of the suspension into a filtering centrifuge, and washing the precipitated mRNA in the filtering centrifuge. The washed precipitated mRNA can then be solubilized in a storage solution (e.g., a solution suitable for lyophilisation) or in a pharmaceutically acceptable liquid (e.g., water for injection).

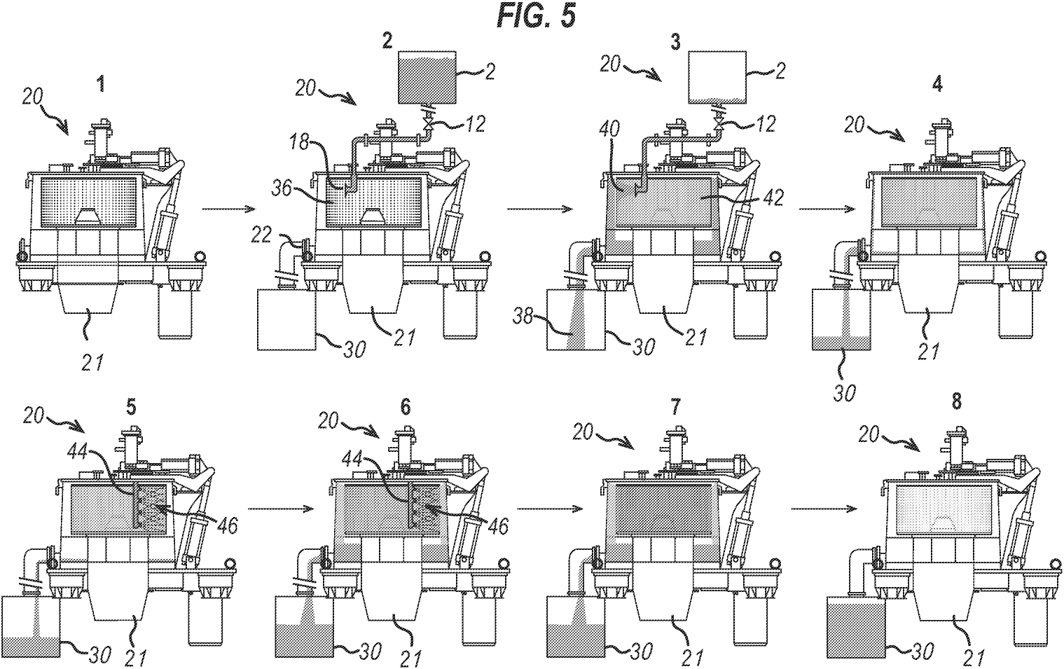

FIG. 4 provides flow chart outlining the steps of an exemplary process of the invention, including additional optional steps (displayed by dashed lines). In some embodiments, the methods of the invention comprise the steps provided in FIG. 4. In some embodiments, the methods of the invention further comprise the optional steps provided in FIG. 4.

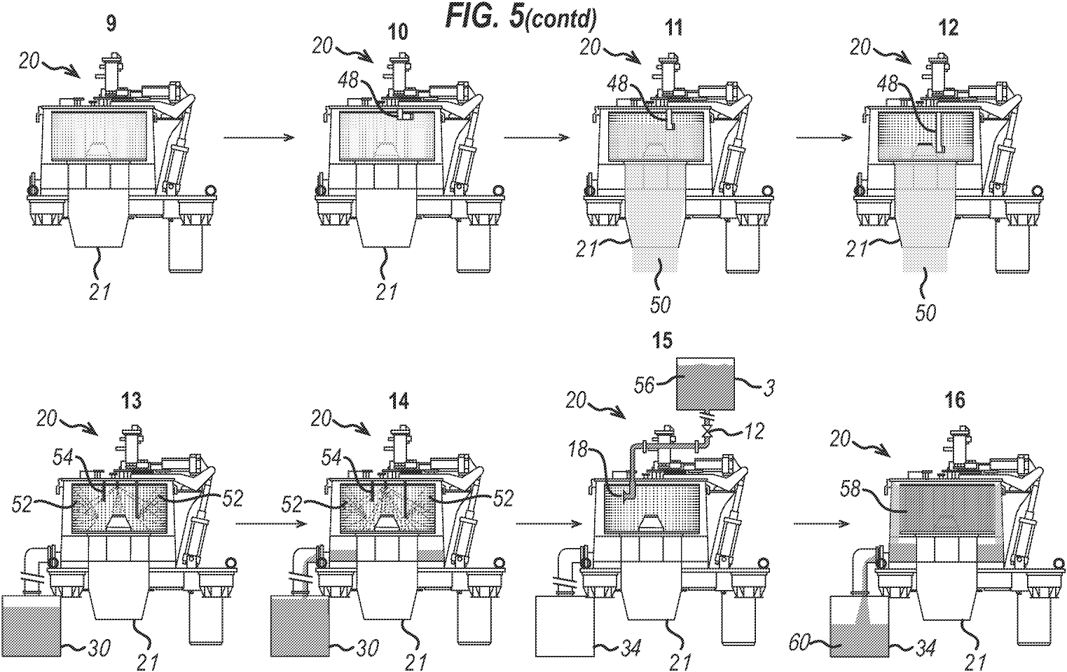

Furthermore, FIG. 5 provides a schematic flow diagram outlining the steps of an exemplary process of the invention carried out on an exemplary system of the invention. The system and process in FIG. 5 is configured only for those embodiments in which the precipitated mRNA is recovered from the filter of the filtering centrifuge as a composition of precipitated mRNA and subsequently solubilised before being collected using a filtering centrifuge to provide purified mRNA. The system comprises: a first vessel (2) for receiving a suspension of precipitated mRNA (40); a second vessel (3) for solubilising the washed precipitated mRNA or for receiving an aqueous medium for solubilising precipitated mRNA; a third vessel (e.g. a waste drum) (30) for collecting contaminants (38); a fourth vessel (34) for receiving purified mRNA (60); a filtering centrifuge (20) comprising a basket or drum (36) having a porous substrate (e.g. a filter), a sample feed port (18), an input nozzle (44), a sample discharge port (22), a sample discharge channel (21), a plough or blade (48) for dislodging retained precipitated mRNA from the filter and one or more sprinklers (54) for distributing rinsing solution. The process displayed in FIG. 5 comprises the following steps [1] through [16] as shown: [1] a filtering centrifuge is provided; [2] a suspension of precipitated mRNA in combination with a filtration aid (40) is provided to a first vessel (2); [3] the suspension of precipitated mRNA (40) is transferred, via a sample feed port (18) from the first vessel (2) into the filtering centrifuge in operation at a first centrifuge speed (e.g. a centrifuge speed exerting a gravitational (g) force of less than 1300 g) such that the precipitated mRNA in combination with a filtration aid is retained (42) on the filter of the filtering centrifuge and contaminants (either soluble or of a size smaller than the filter pore) (38) pass through the filter into the waste drum (30); [4] the centrifuge continues to operate until substantially all the aqueous portion of the suspension of precipitated mRNA has been collected; [5] wash buffer (46) (optionally from a further vessel) is transferred via an input nozzle (44), into the filtering centrifuge in operation at a second centrifuge speed such that the precipitated retained mRNA in combination with a filtration aid is washed by the wash buffer; [6] and [7] the filtering centrifuge continues to operate such that the wash buffer passes through the retained precipitated mRNA in combination with a filtration aid (42) and the filter of the filtering centrifuge carrying with it contaminants (e.g. salt contaminants) into the waste drum (30); [8] and [9] optionally the centrifuge continues operating at the first, second or a third centrifuge speed such that the retained washed precipitated mRNA in combination with a filtration aid is dried; [10]-[12] a plough or blade (48) is deployed and dislodges the retained washed precipitated mRNA in combination with a filtration aid from the filter of the filtering centrifuge operating at a third centrifuge speed such that the washed precipitated mRNA is collected as a composition of precipitated mRNA in combination with a filtration aid (50) via the sample discharge channel (21) (this step can also be performed manually, with the centrifuge not in operation (not shown)); [13] and [14] optionally the filter and basket of the filtering centrifuge can be rinsed with a rinsing buffer (e.g. water comprising NaOH) (52) transferred into the filtering centrifuge via sprinklers (54) (this is known as a clean-in-place (CIP) system) and collected in the waste drum (30); [15] the composition of precipitated mRNA in combination with a filtration aid (50) is solubilised in a solubilisation buffer to provide an aqueous solution of solubilised mRNA in combination with a filtration aid (56) which is transferred into a vessel (3) for receiving solubilised mRNA (the step of solubilisation can also occur inside this vessel (3); [15] the aqueous solution of mRNA in combination with a filtration aid is transferred, via a sample feed port (18) from the vessel (3) into the filtering centrifuge in operation at a fourth centrifuge such that the filtration aid is retained by the filter of the filtering centrifuge and the aqueous mRNA solution passes through the filter into a further vessel (34) for receiving a solution of purified mRNA (60). In some embodiments, the centrifuge is operated at the same centrifuge speed for all of the steps of the process. In some embodiments, the first and second, and optionally the third centrifuge speeds are the same. Exemplary centrifuge speeds are provided in detail below. In particular, steps [2]-[7] of the process occur at a centrifuge speed exerting a gravitational (g) force of less than 1300 g.

In some embodiments, a process of the invention includes one or more steps of preparing in vitro synthesized mRNAs. In other embodiments, the manufacturing of the mRNA through in vitro synthesis is separate from its purification, both physically and temporally. More typically, the purification method of the invention is an integral process of synthesizing the mRNA, i.e., an in vitro synthesis process in accordance with the invention may include one or more purification steps performed in accordance with the present invention.

In some embodiments, a process of the invention includes one or more steps of solubilizing the mRNA after purification. In other embodiments, the purified mRNA is stored and solubilized at a different time. For example, it may be advantageous to ship the purified precipitated mRNA because of its smaller volume before solubilizing.

### mRNA Synthesis

In vitro transcription (IVT) is typically performed with a linear or circular DNA template comprising a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. Accordingly, in some embodiments, the manufacturing the mRNA comprises the steps of performing in vitro transcription (IVT) by mixing (i) a DNA template comprising a suitable promoter and (ii) an RNA polymerase, to generate an impure preparation comprising full-length mRNA which is then subjected to the purification methods disclosed herein. The presence of these IVT is undesirable in the final product and may thus be referred to as impurities and a preparation containing one or more of these impurities may be referred to as an impure preparation.

In particular embodiments, the IVT reaction comprises a two-step process, the first step comprising in vitro transcription of mRNA followed by a purification step in accordance with the present invention, and the second step comprises capping and tailing of the in vitro transcribed mRNA followed by a second purification step in accordance with the present invention. In some embodiments, the IVT reaction is a one step process which results in the in vitro transcription of capped and tailed mRNA. For example, in some embodiments, the in vitro transcription results in the production of capped and tailed mRNA which is subsequently purified. This is accomplished, for example, by using plasmids that comprise a polyT region and/or CleanCap® (i.e. 50 mM m7G(5′)ppp(5′)(2′OMeG)pG in sodium salt form in an aqueous buffer).

In some embodiments, the DNA template is a linear DNA template. In some embodiments, the DNA template is a circular DNA template. In some embodiments, the polymerase is SP6 polymerase. In some embodiments, the mixing further includes mixing a pool of ribonucleotide triphosphates. In some embodiments, the mixing further includes an RNase inhibitor, for example an RNase I inhibitor, RNase A, RNase B, and RNase C.

In some embodiments, the DNA template to be transcribed may be optimized to facilitate more efficient transcription and/or translation. For example, the DNA template may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA template may be optimized regarding cryptic splice sites, mRNA secondary structure, stable free energy of mRNA, repetitive sequences, mRNA instability motif, and/or other elements relevant to mRNA processing and stability; the DNA template may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA template may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which is described in US 20110081708, the contents of which are incorporated herein by reference in its entirety.

In some embodiments, the DNA template includes a 5′ and/or 3′ untranslated region. In some embodiments, a 5′ untranslated region includes one or more elements that affect an mRNA’s stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA’s stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, and citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also disclosed is the inclusion of a sequence encoding human growth hormone (hGH), or a fragment thereof to the 3′ end or untranslated region of the polynucleotide (e.g., mRNA) to further stabilize the polynucleotide. Generally, these features improve the stability and/or pharmacokinetic properties (e.g., half-life) of the polynucleotide relative to the same polynucleotide without such features, and include, for example features made to improve such polynucleotides’ resistance to in vivo nuclease digestion.

In some embodiments, capping of the in vitro synthesized mRNA is performed in a separate reaction. In such a reaction, a 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited tom7G(5′)ppp(5′)(2′OMeG), m7G(5′)ppp(5′)(2′OMeA), m7(3′OMeG)(5′)ppp(5′)(2′OMeG), m7(3′OMeG)(5′)ppp(5′)(2′OMeA), m7G(5′)ppp (5′(A,G(5′)ppp(5′)A and G(5′)ppp(5′)G. In a specific embodiment, the cap structure is m7G(5′)ppp(5′)(2′OMeG). Additional cap structures are described in published U.S. Application No. US 2016/0032356 and U.S. Provisional Application 62/464,327, filed Feb. 27, 2017, which are incorporated herein by reference.

In some embodiments, the manufacturing the mRNA comprises a method for large-scale production of full-length mRNA molecules. In some embodiments, the manufacturing the mRNA comprises a method for producing a composition enriched for full-length mRNA molecules which are greater than 500 nucleotides in length In some embodiments, at least 81%, 82%, 83%, 84%, 85%, 86%, 87%, 88%, 89%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, 99.9% of the purified mRNA molecules are full-length mRNA molecules.

In some embodiments, a composition or a batch includes at least 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more mRNA. In some embodiments, the suspension of precipitated mRNA comprises at least 100 mg, 1 g, 10 g, 100 g, 250 g, 500 g, 1 kg, 10 kg, 100 kg, one metric ton, or ten metric tons, of mRNA or any amount there between. In one embodiment, the suspension of precipitated mRNA comprises at least 250 g of mRNA. In another embodiment, the suspension of precipitated mRNA comprises at least 500 g of mRNA. In a particular embodiments, the suspension of precipitated mRNA comprises greater than 1 kg of mRNA.

In some embodiments, the mRNA molecules are greater than 600, 700, 800, 900, 1000, 2000, 3000, 4000, 5000, 10,000 or more nucleotides in length; also included in the present invention is mRNA having any length in between.

### Precipitation of the mRNA

In accordance with the methods of the invention, an in vitro synthesized mRNA is precipitated to provide a suspension comprising the precipitated mRNA, such that it can be separated from contaminants by means of a filtering centrifuge. The suspension can comprise various contaminants, for example, plasmid DNA and enzymes.

Any and all methods suitable for precipitating mRNA may be used to practice the present invention.

### Agents for Precipitating mRNA

In some embodiments, precipitating the mRNA comprises adding one or more agents that promote precipitation of mRNA, for example one or more of an alcohol, an amphiphilic polymer, a buffer, a salt, and/or a surfactant. In particular embodiments, the one or more agents that promote precipitation of the mRNA are (i) a salt (e.g., a chaotropic salt) and (ii) an alcohol or an amphiphilic polymer.

In some embodiments, the one or more agents that promote precipitation of the mRNA is a salt. High concentrations of salts are known to cause both proteins and nucleic acids to precipitate from an aqueous solution. In some embodiments, more than one salt is used. In some embodiments, a high concentration of salt may be between 1 M and 10 M, inclusive. In some embodiments, a high concentration of salt may be between 2 M and 9 M, inclusive. In some embodiments, a high concentration of salt may be between 2 M and 8 M, inclusive. In some embodiments, a high concentration of salt may be between 2 M and 5 M, inclusive. In some embodiments, a high concentration of salt may be greater than 1 M concentration. In some embodiments, a high concentration of salt may be greater than 2 M concentration. In some embodiments, a high concentration of salt may be greater than 3 M concentration. In some embodiments, a high concentration of salt may be greater than 4 M concentration. In some embodiments, a high concentration of salt may be greater than 5 M concentration. In some embodiments, a high concentration of salt may be greater than 6 M concentration. In some embodiments, a high concentration of salt may be greater than 7 M concentration. In some embodiments, a high concentration of salt may be greater than 8 M concentration. In some embodiments, a single salt is used. In some embodiments, the salt is at a final concentration of 2-4 M, for example of 2.5-3 M. In particular embodiments, the salt is at a final concentration of about 2.7 M.

In some embodiments, the salt may be a calcium salt, an iron salt, a magnesium salt, a potassium salt, a sodium salt, or a combination thereof. Exemplary specific salts suitable for use as agents that promote the precipitation of the mRNA in some embodiments include, but are not limited to, potassium chloride (KCl), sodium chloride (NaCl), lithium chloride (LiCl), calcium chloride (CaCl₂), potassium bromide (KBr), sodium bromide (NaBr), lithium bromide (LiBr). In some embodiments, the denaturing agent the impure preparation is subjected to is potassium chloride (KCl). In some embodiments, KCl is added such that the resulting KCl concentration is about 1 M or greater. In some embodiments, KCl is added such that the resulting KCl concentration is about 2 M or greater, 3 M or greater, 4 M or greater, or 5 M or greater.

In some embodiments, the salt is a chaotropic salt. Chaotropic agents are substances which disrupt the structure of macromolecules such as proteins and nucleic acids by interfering with non-covalent forces such as hydrogen bonds and van der Waals forces. In some embodiments, the chaotropic salt is at a final concentration of 2-4 M, for example of 2.5-3 M. In particular embodiments, the chaotropic salt is at a final concentration of about 2.7 M

In some embodiments, a salt (e.g., a chaotropic salt such as guanidine thiocyanate (GSCN)) is added to an mRNA-containing suspension to denature and solubilize contaminating proteins. Accordingly, in one embodiment, GSCN is the salt in the suspension. This is followed by the addition of an amphiphilic polymer or an alcohol to selectively precipitate mRNA. After mRNA precipitation, the resulting precipitated mRNA is loaded into a filtering centrifuge and retained by the filter which is washed to yield a precipitate that is free of contamination, e.g., short abortive RNA species, long abortive RNA species, dsRNA, plasmid DNA, residual in vitro transcription enzymes, residual salt, and residual solvent. Subsequent dissolution of the precipitated mRNA by a solubilisation buffer, e.g., water, yields purified mRNA composition.

Accordingly, in some embodiments, one agent that promotes precipitation of mRNA comprises a chaotropic salt, for example guanidine thiocyanate (e.g., a solution comprising about 1-5 M guanidine thiocyanate). For example, the solution comprises about 1 M, 1.5 M, 2.0 M, 2.5 M, 3.0 M, 3.5 M, 4.0 M, 4.5 M, or about 5 M of a chaotropic salt, for example GSCN. Examples of suitable GSCN buffers include, for example, an aqueous solution comprising 4 M guanidine thiocyanate, 25 mM sodium citrate pH 6.5, 0.5% N-lauroylsarcosine sodium salt. A further example of a GSCN buffer is an aqueous solution comprising 5 M GSCN in a 10 mM dithiothreitol (DTT) buffer. In some embodiments, GSCN is at a final concentration of 2-4 M. In some embodiments, the GSCN (for example 5 M GSCN-10 mM DTT buffer) is at a final concentration of 2.5-3 M. In particular embodiments, GSCN is at a final concentration of about 2.7 M.

In some embodiments, two agents are used to promote precipitation of mRNA, wherein one agent comprises guanidine thiocyanate (e.g., an aqueous solution of guanidine thiocyanate such as a GSCN buffer) and a second agent comprises a volatile organic solvent such as an alcohol (e.g., ethanol) or an amphiphilic polymer (e.g., polyethylene glycol (PEG)). In embodiments, the two agents are used sequentially or simultaneously. In embodiments, the method includes use of a solution comprising guanidine thiocyanate (e.g., a GSCN buffer) and (i) an alcohol (e.g., absolute ethanol or an aqueous solution of an alcohol such as aqueous ethanol) or (ii) an amphiphilic polymer (e.g., PEG having a molecular weight of about 4000 to about 8000 g/mol, typically at a final concentration of about 10% to about 20% (weight/volume) in an aqueous solution). In a typical embodiment, the solution further comprises a filtration aid (for example a cellulose-based filtration aid, e.g., Solka-Floc). The filtration aid may be present in the final solution at a mass ratio with the precipitated mRNA of about 10:1. In some embodiments, no filtration aid is used when precipitating the mRNA. In some embodiments, the filtration aid is added to the aqueous suspension comprising the precipitated mRNA.

In some embodiments, a one or more agents that promote precipitation of mRNA includes a volatile organic solvent such as an alcohol (e.g., ethanol such as absolute ethanol). In embodiments, a one or more agents that promote precipitation of mRNA is an aqueous solution of an alcohol (e.g., aqueous ethanol). In embodiments, a one or more agents that promote precipitation of mRNA is absolute ethanol.

In some embodiments, the final suspension comprises a volatile organic solvent such as an alcohol.. Suitable alcohols include ethanol, isopropyl alcohol, and benzyl alcohol. Typically, the final suspension comprises the alcohol (e.g., ethanol) at about 50%, 60%, 70%, 80% or 90% weight/volume concentration. In some embodiments, the final suspension comprises alcohol (e.g., ethanol) at less than about 50%, 40%, 30%, 20% or 10% weight/volume concentration. In particular embodiments, the final suspension comprises alcohol (e.g., ethanol) at about 50% weight/volume concentration.

In some embodiments, the suspension is free of volatile organic solvents, in particular free of alcohols, which are highly flammable and therefore pose safety restrictions on the volumes that can be store in a facility. In specific embodiments, the wash buffer comprises an amphiphilic polymer in place of an alcohol, such as ethanol. Suitable amphiphilic polymers for the alcohol-free (and in particular, ethanol-free) methods of the invention are known in the art. In some embodiments, amphiphilic polymer used in the methods herein include pluronics, polyvinyl pyrrolidone, polyvinyl alcohol, polyethylene glycol (PEG), or combinations thereof. In some embodiments, the amphiphilic polymer is selected from one or more of the following: PEG triethylene glycol, tetraethylene glycol, PEG 200, PEG 300, PEG 400, PEG 600, PEG 1,000, PEG 1,500, PEG 2,000, PEG 3,000, PEG 3,350, PEG 4,000, PEG 6,000, PEG 8,000, PEG 10,000, PEG 20,000, PEG 35,000, and PEG 40,000, or combination thereof. In some embodiments, the amphiphilic polymer comprises a mixture of two or more kinds of molecular weight PEG polymers are used. For example, in some embodiments, two, three, four, five, six, seven, eight, nine, ten, eleven, or twelve molecular weight PEG polymers comprise the amphiphilic polymer. Accordingly, in some embodiments, the PEG solution comprises a mixture of one or more PEG polymers. In some embodiments, the mixture of PEG polymers comprises polymers having distinct molecular weights.

In some embodiments, precipitating the mRNA in a suspension comprises one or more amphiphilic polymers. In some embodiments, the precipitating the mRNA in a suspension comprises a PEG polymer. Various kinds of PEG polymers are recognized in the art, some of which have distinct geometrical configurations. PEG polymers suitable for the methods herein include, for example, PEG polymers having linear, branched, Y-shaped, or multi-arm configuration. In some embodiments, the PEG is in a suspension comprising one or more PEG of distinct geometrical configurations. In some embodiments, precipitating mRNA can be achieved using PEG-6000 to precipitate the mRNA. In some embodiments, precipitating mRNA can be achieved using PEG-400 to precipitate the mRNA. In particular embodiments, precipitating mRNA can be achieved using PEG having a molecular weight of about 4000 to about 8000 g/mol, e.g., about 6000 g/mol (e.g. PEG-6000), typically at a final concentration of about 10% to about 20% (weight/volume).

In some embodiments, precipitating mRNA can be achieved using triethylene glycol (TEG) to precipitate the mRNA. In some embodiments, precipitating mRNA can be achieved using triethylene glycol monomethyl ether (MTEG) to precipitate the mRNA. In some embodiments, precipitating mRNA can be achieved using tert-butyl-TEG-O-propionate to precipitate the mRNA. In some embodiments, precipitating mRNA can be achieved using TEG-dimethacrylate to precipitate the mRNA. In some embodiments, precipitating mRNA can be achieved using TEG-dimethyl ether to precipitate the mRNA. In some embodiments, precipitating mRNA can be achieved using TEG-divinyl ether to precipitate the mRNA. In some embodiments, precipitating mRNA can be achieved using TEG-monobutyl ether to precipitate the mRNA. In some embodiments, precipitating mRNA can be achieved using TEG-methyl ether methacrylate to precipitate the mRNA. In some embodiments, precipitating mRNA can be achieved using TEG-monodecyl ether to precipitate the mRNA. In some embodiments, precipitating mRNA can be achieved using TEG-dibenzoate to precipitate the mRNA. Any one of these PEG or TEG based reagents can be used in combination with guanidinium thiocyanate to precipitate the mRNA. The structures of each of these reagents is shown below in Table A.

TABLE A

Non-Organic Solvent Reagents for Purification of mRNA (Precipitation and/or Washing of mRNA)

| | |
|---|---|
| Reageant Name | Structure |
| TEG | 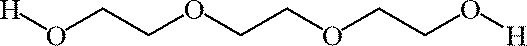 |
| TEG-monomethyl ether | 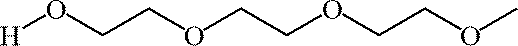 |
| tert-butyl-TEG-O-propionate | 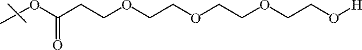 |
| TEG-dimethacrylate | 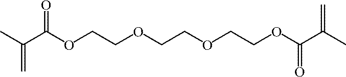 [SMILES: C=C(C)C(=O)OCCOCCOCCOC(=O)C(=C)C] |
| TEG-dimethyl ether | 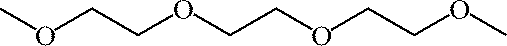 |
| TEG-divinyl ether | 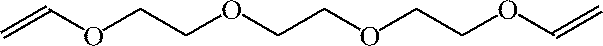 |
| TEG-monobutyl ether | 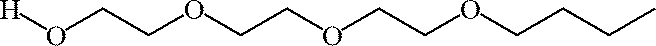 |
| TEG-methyl ether methacrylate | 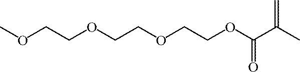 [SMILES: C=C(C)C(=O)OCCOCCOCCOC] |
| TEG-monoderyl ether | 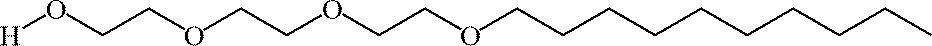 [SMILES: CCCCCCCCCCOCCOCCOCCO] |
| TEG-dibenzoate | 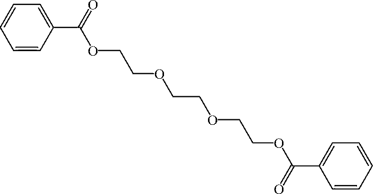 [SMILES: O=C(OCCOCCOCCOC(=O)c1ccccc1)c1ccccc1] |

In some embodiments, precipitating the mRNA in a suspension comprises a PEG polymer, wherein the PEG polymer comprises a PEG-modified lipid. In some embodiments, the PEG-modified lipid is 1,2-dimyristoyl-sn-glycerol, methoxypolyethylene glycol (DMG-PEG-2K). In some embodiments, the PEG modified lipid is a DOPA-PEG conjugate. In some embodiments, the PEG-modified lipid is a poloxamer-PEG conjugate. In some embodiments, the PEG-modified lipid comprises DOTAP. In some embodiments, the PEG-modified lipid comprises cholesterol.

In some embodiments, the mRNA is precipitated in suspension comprising an amphiphilic polymer. In some embodiments, the mRNA is precipitated in a suspension comprising any of the aforementioned PEG reagents. In some embodiments, PEG is in the suspension at about 10% to about 100% weight/volume concentration. For example, in some embodiments, PEG is present in the suspension at about 5%, 10%, 15%, 20%, 25%, 30%, 35%, 40%, 45%, 50%, 55%, 60%, 65%, 70%, 75%, 80%, 85%, 90%, 95%, 100% weight/volume concentration, and any values there between. In some embodiments, PEG is present in the suspension at about 5% weight/volume concentration. In some embodiments, PEG is present in the suspension at about 6% weight/volume concentration. In some embodiments, PEG is present in the suspension at about 7% weight/volume concentration. In some embodiments, PEG is present in the suspension at about 8% weight/volume concentration. In some embodiments, PEG is present in the suspension at about 9% weight/volume concentration. In some embodiments, PEG is present in the suspension at about 10% weight/volume concentration. In some embodiments, PEG is present in the suspension at about 12% weight/volume concentration. In some embodiments, PEG is present in the suspension at about 15% weight/volume. In some embodiments, PEG is present in the suspension at about 18% weight/volume. In some embodiments, PEG is present in the suspension at about 20% weight/volume concentration. In some embodiments, PEG is present in the suspension at about 25% weight/volume concentration. In some embodiments, PEG is present in the suspension at about 30% weight/volume concentration. In some embodiments, PEG is present in the suspension at about 35% weight/volume concentration. In some embodiments, PEG is present in the suspension at about 40% weight/volume concentration. In some embodiments, PEG is present in the suspension at about 45% weight/volume concentration. In some embodiments, PEG is present in the suspension at about 50% weight/volume concentration. In some embodiments, PEG is present in the suspension at about 55% weight/volume concentration. In some embodiments, PEG is present in the suspension at about 60% weight/volume concentration. In some embodiments, PEG is present in the suspension at about 65% weight/volume concentration. In some embodiments, PEG is present in the suspension at about 70% weight/volume concentration. In some embodiments, PEG is present in the suspension at about 75% weight/volume concentration. In some embodiments, PEG is present in the suspension at about 80% weight/volume concentration. In some embodiments, PEG is present in the suspension at about 85% weight/volume concentration. In some embodiments, PEG is present in the suspension at about 90% weight/volume concentration. In some embodiments, PEG is present in the suspension at about 95% weight/volume concentration. In some embodiments, PEG is present in the suspension at about 100% weight/volume concentration.

In some embodiments, precipitating the mRNA in a suspension comprises a volume:volume ratio of PEG to total mRNA suspension volume of about 0.1 to about 5.0. For example, in some embodiments, PEG is present in the mRNA suspension at a volume:volume ratio of about 0.1, 0.2, 0.3, 0.4, 0.5, 0.6, 0.7, 0.8, 0.9, 1.0, 1.25, 1.5, 1.75, 2.0, 2.25, 2.5, 2.75, 3.0, 3.25, 3.5, 3.75, 4.0, 4.25, 4.5, 4.75, 5.0. Accordingly, in some embodiments, PEG is present in the mRNA suspension at a volume:volume ratio of about 0.1. In some embodiments, PEG is present in the mRNA suspension at a volume:volume ratio of about 0.2. In some embodiments, PEG is present in the mRNA suspension at a volume:volume ratio of about 0.3. In some embodiments, PEG is present in the mRNA suspension at a volume:volume ratio of about 0.4. In some embodiments, PEG is present in the mRNA suspension at a volume:volume ratio of about 0.5. In some embodiments, PEG is present in the mRNA suspension at a volume:volume ratio of about 0.6. In some embodiments, PEG is present in the mRNA suspension at a volume:volume ratio of about 0.7. In some embodiments, PEG is present in the mRNA suspension at a volume:volume ratio of about 0.8. In some embodiments, PEG is present in the mRNA suspension at a volume:volume ratio of about 0.9. In some embodiments, PEG is present in the mRNA suspension at a volume:volume ratio of about 1.0. In some embodiments, PEG is present in the mRNA suspension at a volume:volume ratio of about 1.25. In some embodiments, PEG is present in the mRNA suspension at a volume:volume ratio of about 1.5. In some embodiments, PEG is present in the mRNA suspension at a volume:volume ratio of about 1.75. In some embodiments, PEG is present in the mRNA suspension at a volume:volume ratio of about 2.0. In some embodiments, PEG is present in the mRNA suspension at a volume:volume ratio of about 2.25. In some embodiments, PEG is present in the mRNA suspension at a volume:volume ratio of about 2.5. In some embodiments, PEG is present in the mRNA suspension at a volume:volume ratio of about 2.75. In some embodiments, PEG is present in the mRNA suspension at a volume:volume ratio of about 3.0. In some embodiments, PEG is present in the mRNA suspension at a volume:volume ratio of about 3.25. In some embodiments, PEG is present in the mRNA suspension at a volume:volume ratio of about 3.5. In some embodiments, PEG is present in the mRNA suspension at a volume:volume ratio of about 3.75. In some embodiments, PEG is present in the mRNA suspension at a volume:volume ratio of about 4.0. In some embodiments, PEG is present in the mRNA suspension at a volume:volume ratio of about 4.25. In some embodiments, PEG is present in the mRNA suspension at a volume:volume ratio of about 4.50. In some embodiments, PEG is present in the mRNA suspension at a volume:volume ratio of about 4.75. In some embodiments, PEG is present in the mRNA suspension at a volume:volume ratio of about 5.0. In particular embodiments, PEG is present in the mRNA suspension at a volume:volume ratio of about 1.0, about 1.5 or about 2.0.

In some embodiments, a reaction volume for mRNA precipitation comprises GSCN and PEG. In particular embodiments, a reaction volume for mRNA precipitation comprises GSCN and PEG having a molecular weight of about 4000 to about 8000 g/mol, e.g., about 6000 g/mol (e.g. PEG-6000). GSCN is typically at a final concentration between 2 M and 4 M. PEG is typically at a final concentration of about 10% to about 20% (weight/volume).

In some embodiments, the mRNA is precipitated in a suspension comprising GSCN at a final concentration of between about 2-4 M; PEG having a molecular weight of about 4000 to about 8000 g/mol, e.g., about 6000 g/mol (e.g. PEG-6000) at a final concentration of between about 5% and about 20% (weight/volume); and a filtration aid (for example a cellulose-based filtration aid) at a mass ratio with the precipitated mRNA of about 2:1; about 5:1; about 10:1 or about 15:1. In some embodiments, the mRNA is precipitated in a suspension comprising GSCN at a final concentration of about 2.5-3 M; PEG having a molecular weight of about 6000 g/mol (e.g. PEG-6000) at a final concentration of between about 10% and about 15% (weight/volume); and a filtration aid (for example a cellulose-based filtration aid) at a mass ratio with the precipitated mRNA of about 10:1. In particular embodiments, the mRNA is precipitated in a suspension comprising GSCN at a final concentration of about 2.7 M; PEG having a molecular weight of about 6000 g/mol (e.g. PEG-6000) at a final concentration of about 12% (weight/volume); and a filtration aid (for example a cellulose-based filtration aid, e.g., Solka-Floc) at a mass ratio with the precipitated mRNA of about 10:1. As shown in the examples, suspensions comprising these concentrations of mRNA, salt and PEG achieve highly effective purification of the mRNA in the methods of the present invention.

In some embodiments, MTEG can be used in place of PEG to provide a suspension of precipitated mRNA. In particular embodiments, MTEG is used for this purpose at a final concentration of about 15% to about 45% weight/volume. In some embodiments, the suspension comprises MTEG at a final concentration of about 20% to about 40% weight/volume. In some embodiments, the suspension comprises MTEG at a final concentration of about 20% weight/volume. In some embodiments, the suspension comprises MTEG at a final concentration of about 25% weight/volume. In some embodiments, the suspension comprises MTEG at a final concentration of about 30% weight/volume. In some embodiments, the suspension comprises MTEG at a final concentration of about 35% weight/volume. In some embodiments, the suspension comprises MTEG at a final concentration of less than 35% weight/volume. The rest of the conditions used in MTEG-induced precipitation are the same as those used in the PEG-induced precipitation. Particularly suitable for efficient recovery of mRNA in the methods of the present invention is a suspension comprising mRNA, GSCN and MTEG, with MTEG at a final concentration of about 25%, in addition to a filtration aid (for example cellulose-based filtration aid) at a mass ratio with the precipitated mRNA of about 10:1.

For example, GSCN can be provided as a 4-8 M solution (e.g. in a 10 mM DTT buffer), which is then combined with the mRNA (typically at a concentration 1 mg/ml) and MTEG (available at a purity of ≥97.0%) to prepare a suspension of precipitated mRNA. In some embodiments, the suspension comprises precipitated mRNA, a chaotropic salt, for example GSCN, and MTEG at a volume ratio of 1:2-3:1-2. In some embodiments, the suspension comprises precipitated mRNA, a chaotropic salt, for example GSCN, and MTEG at a volume ratio of 1:2-2.5:1-2. In some embodiments, the suspension comprises precipitated mRNA, a chaotropic salt, for example GSCN, and MTEG at a volume ratio of 1:2.3:1-2. In particular embodiments, the suspension comprises precipitated mRNA, GSCN, and MTEG at a ratio of 1:2.3:2. In particular embodiments, the suspension comprises precipitated mRNA, GSCN, and MTEG at a volume ratio of 1:2.3:1.7. In particular embodiments, the suspension comprises precipitated mRNA, GSCN, and MTEG at a ratio of 1:2.3:1. As shown in the examples, a suspension comprising mRNA, GSCN and MTEG in volume ratios of 1:2.3:1, 1:2.3:1.7 and 1:2.3:2 is particularly suitable for achieving purified mRNA in the methods of the present invention in combination with an MTEG wash solution at a final concentration of about 95% - this combination of steps ensures efficient purification and recovery of mRNA.

In some embodiments, two agents are used to promote precipitation of mRNA, wherein one agent comprises guanidine thiocyanate (e.g., an aqueous solution of guanidine thiocyanate such as a GSCN buffer) and a second agent comprises an amphiphilic polymer (e.g., PEG and/or MTEG). In some embodiments, the two agents are used sequentially or simultaneously. In some embodiments, the method includes use of a solution comprising guanidine thiocyanate (e.g., a GSCN buffer) and an amphiphilic polymer (e.g., PEG and/or MTEG).

In some embodiments, a precipitating step comprises the use of a chaotropic salt (e.g., guanidine thiocyanate) and/or an amphiphilic polymer (e.g., PEG and/or MTEG) and/or an alcohol solvent (e.g., absolute ethanol or an aqueous solution of alcohol such as an aqueous ethanol solution). Accordingly, in some embodiments, the precipitating step comprises the use of a chaotropic salt and an amphiphilic polymer, such as GSCN and PEG and/or MTEG, respectively.

In some embodiments, the suspension for precipitating the mRNA comprises precipitated mRNA, a salt and MTEG. In some embodiments, the suspension is free of alcohol, for example ethanol.

### Filtration Aids (Including Dispersants)

In some embodiments, a filtration aid is used in a method described herein (e.g., during centrifugation). A filtration aid may be used when purifying precipitated mRNA using a filtering centrifuge. The filtration aid may assist in retaining precipitated mRNA on the filter of a filtering centrifuge and may facilitate removal of the retained mRNA from the surface of the filter of a filtering centrifuge.

In some embodiments, a filtration aid is a dispersant. In some embodiments, the precipitated mRNA composition includes at least one dispersant, e.g. one or more of ash, clay, diatomaceous earth, glass beads, plastic beads, polymers, polymer beads (e.g., polypropylene beads, polystyrene beads), salts (e.g., cellulose salts), sand, and sugars. In some embodiments, the polymer is a naturally occurring polymer, e.g. cellulose (for example, powdered cellulose fibre).

In some embodiments, a filtration aid suitable for use with the methods of the present invention is cellulose-based. In embodiments, a cellulose filtration aid is powdered cellulose fiber (e.g., Solka-Floc® or Sigmacell Cellulose 20). In embodiments, a cellulose filtration aid is a powdered cellulose fiber such as Solka-Floc® 100 NF or Sigmacell Cellulose Type 20. In some embodiments, the cellulose-based filtration aid has a particle size of about 20 µm.

In some embodiments, the precipitated mRNA and filtration aid (for example powdered cellulose fibre such as Solka Floc) are at a mass ratio of 1:2; 1:5; 1:10 or 1:15. In particular embodiments, the precipitated mRNA and filtration aid (for example powdered cellulose fibre such as Solka Floc) are at a mass ratio of 1:10.

In some embodiments, precipitation of mRNA is performed in the absence of a filtration aid. In some embodiments, the precipitated mRNA composition does not comprise a filtration aid.

In some embodiments, precipitation of mRNA is performed in the presence of at least one filtration aid.

In some embodiments, a filtration aid is added to the slurry obtained following the precipitation of mRNA.

In some embodiments, a purification method may further include one or more steps for separating the filtration aid from the retained precipitated mRNA. The method may further include a step of solubilizing the precipitated and purified mRNA from the cake using an aqueous medium, e.g., water, and collecting the solubilised mRNA, while retaining the filtration aid on a filter, for example using a filtering centrifuge.

### Washing of the Retained Precipitated mRNA

### Composition of the Wash Buffer

The method of purifying mRNA comprises washing the retained precipitated mRNA to remove the salt required for the precipitation step and to remove any contaminants in the suspension of precipitated mRNA. The step of washing the retained precipitated mRNA involves washing the retained precipitated mRNA with a wash buffer. The terms “wash buffer” and “wash solution” can be used interchangeably herein.

In some embodiments, the wash buffer comprises one or more of an alcohol, an amphiphilic polymer, a buffer, a salt, and/or a surfactant. In some embodiments, the wash buffer comprises an alcohol or an amphiphilic polymer.

In some embodiments, the wash buffer comprises a volatile organic solvent, e.g. an alcohol. Suitable alcohols include ethanol, isopropyl alcohol, and benzyl alcohol. Typically, the wash buffer comprises the alcohol (e.g. ethanol) at about at least 50%, 60%, 70%, 80% or 90% weight/volume concentration. In some embodiments, the wash buffer comprises alcohol (e.g. ethanol) at about 50%, 60%, 70%, 80% or 90% weight/volume concentration. In particular embodiments, the wash buffer comprises alcohol at about 80% weight/volume concentration. In particular embodiments, the alcohol in the wash buffer is ethanol.

In some embodiments, the wash buffer is free of volatile organic solvents, in particular free of alcohols, which are highly flammable and therefore pose safety restrictions on the volumes that can be store in a facility. In specific embodiments, the wash buffer comprises an amphiphilic polymer in place of an alcohol, such as ethanol. Suitable amphiphilic polymers for the alcohol-free (and in particular, ethanol-free) methods of the invention are selected from pluronics, polyvinyl pyrrolidone, polyvinyl alcohol, polyethylene glycol (PEG), triethylene glycol monomethyl ether (MTEG), or combinations thereof. Polyethylene glycol (PEG) (e.g., PEGs having a low molecular weight, in particular of about 200-600 g/mol) and especially triethylene glycol monomethyl ether (MTEG) are particularly suitable for practising the alcohol-free (and in particular, ethanol free) embodiments of the invention.

In some embodiments, the amphiphilic polymer is a polyethylene glycol (PEG). Accordingly, in some embodiments, a PEG solution (“PEG wash solution”) is used for washing the retained mRNA. The PEG wash solution comprises triethylne glycol, tetraethylene glycol, PEG 200, PEG 300, PEG 400, PEG 600, PEG 1,000, PEG 1,500, PEG 2,000, PEG 3,000, PEG 3,350, PEG 4,000, PEG 6,000, PEG 8,000, PEG 10,000, PEG 20,000, PEG 35,000, and PEG 40,000, or combination thereof. In some embodiments, the PEG wash solution comprises triethylene glycol. In some embodiments, the PEG wash solution comprises tetraethylene glycol. In some embodiments, the PEG wash solution comprises PEG 200. In some embodiments, the PEG solution comprises PEG 300. In some embodiments, the wash PEG wash solution comprises PEG 400. In some embodiments, the PEG wash solution comprises PEG 600. In some embodiments, the PEG wash solution comprises PEG 1,000. In some embodiments, the PEG wash solution comprises PEG 1,500. In some embodiments, the PEG wash solution comprises PEG 2,000. In some embodiments, the PEG wash solution comprises PEG 3,000. In some embodiments, the PEG wash solution comprises PEG 3,350. In some embodiments, the PEG wash solution comprises PEG 4,000. In some embodiments, the PEG wash solution comprises PEG 6,000. In some embodiments, the PEG wash solution comprises PEG 8,000. In some embodiments, the PEG wash solution comprises PEG 10,000. In some embodiments, the PEG wash solution comprises PEG 20,000. In some embodiments, the PEG wash solution comprises PEG 35,000. In some embodiments, the PEG wash solution comprises PEG 40,000.

In some embodiments, the molecular weight of the PEG in the wash solution is about 100 to about 1000 g/mol. In some embodiments, the molecular weight of the PEG in the wash solution is about 200 to about 6000 g/mol. In some embodiments, the molecular weight of the PEG in the wash solution is about 100 g/mol; 200 g/mol (e.g. PEG 200); 300 g/mol (e.g. PEG 300); 400 g/mol (e.g. PEG 400); 500 g/mol; 600 g/mol (e.g. PEG 600) or 1000 g/mol (e.g. PEG 1000). In particular embodiments, the molecular weight of the PEG in the wash solution is about 400 g/mol (e.g. PEG 400).

In some embodiments, washing the precipitated mRNA includes one or more washes comprising PEG having a viscosity of 90 centistrokes or less. In some embodiments, the PEG used to wash the precipitated mRNA has a viscosity of 80 centistrokes or less. In some embodiments, the PEG used to wash the precipitated mRNA has a viscosity of 70 centistrokes or less. In some embodiments, the PEG used to wash the precipitated mRNA has a viscosity of 60 centistrokes or less. In some embodiments, the PEG used to wash the precipitated mRNA has a viscosity of 50 centistrokes or less. In some embodiments, the PEG used to wash the precipitated mRNA has a viscosity of 40 centistrokes or less. In some embodiments, the PEG used to wash the precipitated mRNA has a viscosity of 30 centistrokes or less. In some embodiments, the PEG used to wash the precipitated mRNA has a viscosity of 20 centistrokes or less. In some embodiments, the PEG used to wash the precipitated mRNA has a viscosity of 10 centistrokes or less. The viscosity of a liquid solution can be measured using methods well known in the art, for example using a viscometer, at room temperature (for example between about 18 and 25° C.).

In some embodiments, washing the precipitated mRNA can be achieved using triethylene glycol (TEG). In some embodiments, washing the precipitated mRNA can be achieved using triethylene glycol monomethyl ether (MTEG). In some embodiments, washing the precipitated mRNA can be achieved using tert-butyl-TEG-O-propionate. In some embodiments, washing the precipitated mRNA can be achieved using TEG-dimethacrylate. In some embodiments, washing the precipitated mRNA can be achieved using TEG-dimethyl ether. In some embodiments, washing the precipitated mRNA can be achieved using TEG-divinyl ether. In some embodiments, washing the precipitated mRNA can be achieved using TEG-monobutyl. In some embodiments, washing the precipitated mRNA can be achieved using TEG-methyl ether methacrylate. In some embodiments, washing the precipitated mRNA can be achieved using TEG-monodecyl ether. In some embodiments, washing the precipitated mRNA can be achieved using TEG-dibenzoate. The structures of each of these reagents are shown above in Table A.

In some embodiments, the PEG in the PEG wash solution comprises a PEG-modified lipid. In some embodiments, the PEG in the PEG wash solution is the PEG-modified lipid 1,2-dimyristoyl-sn-glycerol, methoxypolyethylene glycol (DMG-PEG-2K). In some embodiments, the PEG modified lipid is a DOPA-PEG conjugate. In some embodiments, the PEG-modified lipid is a poloxamer-PEG conjugate. In some embodiments, the PEG-modified lipid comprises DOTAP. In some embodiments, the PEG-modified lipid comprises cholesterol.

In some embodiments, the PEG wash solution comprises a mixture of two or more kinds of molecular weight PEG polymers. For example, in some embodiments, two, three, four, five, six, seven, eight, nine, ten, eleven, or twelve molecular weight PEG polymers comprise the PEG wash solution. Accordingly, in some embodiments, the PEG wash solution comprises a mixture of one or more PEG polymers. In some embodiments, the mixture of PEG polymers comprises polymers having distinct molecular weights.

The PEG used in the PEG wash solution can have various geometrical configurations. For example, suitable PEG polymers include PEG polymers having linear, branched, Y-shaped, or multi-arm configuration. In some embodiments, the PEG is in a suspension comprising one or more PEG of distinct geometrical configurations.

In some embodiments, PEG in the wash solution is present at about 10% to about 100% weight/volume concentration. In some embodiments, the PEG in the wash solution is present at about 50% to about 95% weight/volume concentration. For example, in some embodiments, PEG is present in the wash solution at about 10%, 15%, 20%, 25%, 30%, 35%, 40%, 45%, 50%, 55%, 60%, 65%, 70%, 75%, 80%, 85%, 90%, 95%, 100% weight/volume concentration, and any values there between. In some embodiments, PEG is present in the wash solution at about 10% weight/volume concentration. In some embodiments, PEG is present in the wash solution at about 15% weight/volume. In some embodiments, PEG is present in the wash solution at about 20% weight/volume concentration. In some embodiments, PEG is present in the wash solution at about 25% weight/volume concentration. In some embodiments, PEG is present in the wash solution at about 30% weight/volume concentration. In some embodiments, PEG is present in the wash solution at about 35% weight/volume concentration. In some embodiments, PEG is present in the wash solution at about 40% weight/volume concentration. In some embodiments, PEG is present in the wash solution at about 45% weight/volume concentration. In some embodiments, PEG is present in the wash solution at about 50% weight/volume concentration. In some embodiments, PEG is present in the wash solution at about 55% weight/volume concentration. In some embodiments, PEG is present in the wash solution at about 60% weight/volume concentration. In some embodiments, PEG is present in the wash solution at about 65% weight/volume concentration. In some embodiments, PEG is present in the wash solution at about 70% weight/volume concentration. In some embodiments, PEG is present in the wash solution at about 75% weight/volume concentration. In some embodiments, PEG is present in the wash solution at about 80% weight/volume concentration. In some embodiments, PEG is present in the wash solution at about 85% weight/volume concentration. In some embodiments, PEG is present in the wash solution at about 90% weight/volume concentration. In some embodiments, PEG is present in the wash solution at about 95% weight/volume concentration. In some embodiments, PEG is present in the wash solution at about 100% weight/volume concentration. In particular embodiments, the PEG is present in the wash solution at about 90% weight/volume concentration.

In some embodiments, the wash buffer comprises PEG-400 at a concentration of about between 80 and 100%. Accordingly, in some embodiments, the wash buffer comprises PEG-400 at a concentration of about 80%. In some embodiments, the wash buffer comprises PEG-400 at a concentration of about 85%. In some embodiments, the wash buffer comprises PEG-400 at a concentration of about 90%. In some embodiments, the wash buffer comprises PEG-400 at a concentration of about 95%. In some embodiments, the wash buffer comprises PEG-400 at a concentration of about 100%.

In some embodiments, PEG is present in the wash solution at about 90% to about 100% weight/volume concentration. In particular embodiments, the PEG (for example PEG-400) is present in the wash solution at about 90% weight/volume concentration. As shown in the examples, a final concentration of PEG having a molecular weight of about 400 g/mol (e.g. PEG-400) of about 95% resulted in a high yield and highly pure mRNA samples in the methods of the invention.

In some embodiments, the precipitated mRNA is washed in a solution comprising an amphiphilic polymer. In some embodiments, the amphiphilic polymer is MTEG. In some embodiments, MTEG is present in the wash solution at between about 75% and about 95% weight/volume concentration. In some embodiments, MTEG is present in the wash solution at about 75%, about 80%, about 85%, about 90% or about 95% weight/volume concentration. In some embodiments, MTEG is present in the wash solution at about 90% to about 100% by weight/volume concentration. In particular embodiments, MTEG is present in the wash solution at about 95% by weight/volume concentration. As shown in the examples, a final concentration of MTEG of about 95% weight/volume achieved highly efficient recovery of the mRNA in the methods of the invention.

In some embodiments, the wash solution, for example comprising PEG or MTEG, comprises a non-aqueous component, such as, for example, ethanol, isopropyl alcohol or benzyl alcohol. In some embodiments, the wash solution used to wash the captured mRNA is aqueous. Accordingly, in some embodiments, the wash solution is free of non-aqueous components, in particular volatile organic solvent such as alcohol, e.g., ethanol, isopropyl alcohol, or benzyl alcohol.

In come embodiments, the precipitated mRNA can be washed 1, 2, 3, 4, 5, 6, 7, 8, 9, 10, or more than 10 times. Accordingly, in some embodiments, the precipitated mRNA is washed with a solution, for example comprising PEG or MTEG, one time. In some embodiments, the precipitated mRNA is washed with a solution, for example comprising PEG or MTEG, two times. In some embodiments, the precipitated mRNA is washed with a solution, for example comprising PEG or MTEG, three times. In some embodiments, the precipitated mRNA is washed with a solution, for example comprising PEG or MTEG, four times. In some embodiments, the precipitated mRNA is washed with a solution, for example comprising PEG or MTEG, five times. In some embodiments, the precipitated mRNA is washed with a solution, for example comprising PEG or MTEG, six times. In some embodiments, the precipitated mRNA is washed with a solution, for example comprising PEG or MTEG, seven times. In some embodiments, the precipitated mRNA is washed with a solution, for example comprising PEG or MTEG, eight times. In some embodiments, the precipitated mRNA is washed with a solution, for example comprising PEG or MTEG, nine times. In some embodiments, the precipitated mRNA is washed with a solution, for example comprising PEG or MTEG, ten times. In some embodiments, the precipitated mRNA is washed with a solution, for example comprising PEG or MTEG, more than ten times.

In some embodiments, the wash step comprises multiple rinse cycles using a solution comprising an amphiphilic polymer (e.g., polyethylene glycol or MTEG). In some embodiments, the wash step comprises multiple rinses using a solution comprising one or more distinct amphiphilic polymers. In some embodiments, the wash step may be carried out by multiple rinse cycles using a solution comprising about 10% to about 100% amphiphilic polymer. In certain embodiments, the multiple rinse cycles comprise 2 cycles, 3 cycles, 4 cycles, 5 cycles, 6 cycles, 7 cycles, 8 cycles, 9 cycles, 10 cycles or more than 10 cycles.

### Volume of Wash Buffer

As outlined above, the methods of the present invention allow efficient and clinical grade purification of mRNA using reduced volumes of wash buffer in comparison to the methods of the prior art. Therefore, an advantage of the methods of the invention is that the methods use less wash buffer, allowing for more cost and time effective methods for purification of mRNA on a larger commercial scale. As demonstrated in the examples, the methods of the invention, using reduced centrifuge speeds exerting lower force on the precipitated mRNA, require lower volumes of wash buffer to achieve an equivalent or improved mRNA purity compared to the prior art.

In some embodiments, the volume of wash buffer for washing the retained precipitated mRNA is between about 0.5 L/g mRNA and about 8 L/g mRNA. In some embodiments, the volume of wash buffer for washing the retained precipitated mRNA is between about 0.5 L/g mRNA and about 7 L/g mRNA. In some embodiments, the volume of wash buffer for washing the retained precipitated mRNA is between about 0.5 L/g mRNA and about 6 L/g mRNA. In some embodiments, the volume of wash buffer for washing the retained precipitated mRNA is between about 0.5 L/g mRNA and about 5 L/g mRNA. In some embodiments, the volume of wash buffer for washing the retained precipitated mRNA is between about 0.5 L/g mRNA and about 4 L/g mRNA. In some embodiments, the volume of wash buffer for washing the retained precipitated mRNA is between about 0.5 L/g mRNA and about 3 L/g mRNA. In some embodiments, the volume of wash buffer for washing the retained precipitated mRNA is between about 0.5 L/g mRNA and about 2.5 L/g mRNA. In some embodiments, the volume of wash buffer for washing the retained precipitated mRNA is between about 0.5 L/g mRNA and about 2 L/g mRNA. In some embodiments, the volume of wash buffer for washing the retained precipitated mRNA is between about 0.5 L/g mRNA and about 1.5 L/g mRNA. In some embodiments, the volume of wash buffer for washing the retained precipitated mRNA is between about 0.5 L/g mRNA and about 1 L/g mRNA. In particular embodiments, the volume of wash buffer for washing the retained precipitated mRNA is about 0.5 L/g or less.

In some embodiments, the volume of wash buffer for washing the retained precipitated mRNA is less than 8 L/g mRNA. In some embodiments, the volume of wash buffer for washing the retained precipitated mRNA is less than 7 L/g mRNA. In some embodiments, the volume of wash buffer for washing the retained precipitated mRNA is less than 6 L/g mRNA. In some embodiments, the volume of wash buffer for washing the retained precipitated mRNA is less than 5 L/g mRNA. In some embodiments, the volume of wash buffer for washing the retained precipitated mRNA is less than 4 L/g mRNA. In some embodiments, the volume of wash buffer for washing the retained precipitated mRNA is less than 3 L/g mRNA. In some embodiments, the volume of wash buffer for washing the retained precipitated mRNA is less than 2 L/g mRNA. In some embodiments, the volume of wash buffer for washing the retained precipitated mRNA is less than 1.5 L/g mRNA. In some embodiments, the volume of wash buffer for washing the retained precipitated mRNA is less than 1 L/g mRNA. In some embodiments, the volume of wash buffer for washing the retained precipitated mRNA is less than 0.5 L/g mRNA.

In some embodiments, the volume of wash buffer for washing the retained precipitated mRNA is about 1.5, 1 or 0.5 L/g mRNA. In particular embodiments, the volume of wash buffer for washing the retained precipitated mRNA is about 0.5 L/g mRNA or less.

In some embodiments, the manufacturing of the mRNA comprises in vitro transcription (IVT) synthesis of the mRNA. In some embodiments, the manufacturing of the mRNA further comprises a separate step of 3′-tailing of the mRNA. In some embodiments, the separate step of 3′-tailing of the mRNA comprises 5′-capping of the mRNA. In some embodiments, IVT synthesis of the mRNA comprises 5′-capping and/or 3′-tailing of the mRNA. In some embodiments, steps (a) through (d) of the method of the invention are performed after IVT synthesis of the mRNA. In some embodiments, steps (a) through (d) of the method of the invention are performed after IVT synthesis of the mRNA and again after the separate step of 3′tailing of the mRNA. In some embodiments, steps (a) through (d) of the method of the invention are performed after IVT synthesis of the mRNA and again after the separate step of 5′-capping of the mRNA.

In some embodiments, the volume of wash buffer for washing the retained precipitated mRNA is different after each of the steps of the mRNA manufacture process (e.g. after IVT synthesis, 5′-capping and/or 3′-tailing of the mRNA). In some embodiments, the volume of wash buffer for washing the retained precipitated mRNA is the same each of the different steps of the mRNA manufacture process (e.g. after IVT synthesis, 5′-capping and/or 3′-tailing of the mRNA).

The volume of wash buffer may depend on the total amount of mRNA that is to be purified in a single run of the purification method of the invention, i.e., each of steps (a) through (d) is performed only once. In some embodiments, the volume of wash buffer for washing the retained precipitated mRNA after IVT synthesis is less than 8 L/g mRNA, e.g., less than 6 L/g mRNA or less than 5 L/g mRNA. In some embodiments, the volume of wash buffer for washing the retained precipitated mRNA after IVT synthesis is between about 0.5 L/g mRNA and about 4 L/g mRNA. In particular embodiments, the volume of wash buffer for washing the retained precipitated mRNA after IVT synthesis is between about 0.5 L/g mRNA and about 1.5 L/g mRNA, for example about 0.5 L/g mRNA.

In some of embodiments, more than a single run of the purification method of the invention is performed. For example, in certain embodiments, steps (a) through (d) are performed a first time on mRNA obtained from an IVT synthesis reaction. The purified mRNA obtained after performing the method for the first time may then subjected to capping reaction, and the resulting capped mRNA is purified by performing steps (a) through (d) for a second time. In a particular embodiments, a tailing reaction is performed at the same time as the capping reaction is performed. Alternatively, the purified mRNA obtained after performing the method for the first time may then subjected to tailing reaction, and the resulting tailed mRNA is purified by performing steps (a) through (d) for a second time. In a particular embodiments, a capping reaction is performed at the same time as the tailing reaction is performed.

Accordingly, In some embodiments, the total volume of wash buffer for washing the retained precipitated mRNA after IVT synthesis and/or after the separate step of 3′-tailing of the mRNA is less than 8 L/g mRNA, e.g., less than 6 L/g mRNA or less than 5 L/g mRNA. In some embodiments, the total volume of wash buffer for washing the retained precipitated mRNA after IVT synthesis and/or after the separate step of 3′-tailing of the mRNA is between about 0.5 L/g mRNA and about 4 L/g mRNA. In particular embodiments, the total volume of wash buffer for washing the retained precipitated mRNA after IVT synthesis and/or after the separate step of 3′-tailing of the mRNA is between about 0.5 L/g mRNA and about 1.5 L/g mRNA, for example about 1 L/g mRNA. In particular embodiments, the volume of wash buffer for washing the retained precipitated mRNA after IVT synthesis is about 0.5 L/g mRNA. In a particular embodiment, the volume of wash buffer for washing the retained precipitated mRNA after the separate step of 3′-tailing and/or capping of the mRNA is about 0.5 L/g mRNA. In a specific embodiment, the total volume of wash buffer for washing the retained precipitated mRNA after IVT synthesis and after the separate step of 3′-tailing and/or 5′-capping of the mRNA is about 1 L/g mRNA.

### Recovering the Washed Retained Precipitated mRNA

Accordingly, the method of purifying mRNA of the present invention includes a step of recovering the retained precipitated mRNA from the filter of the filtering centrifuge. The recovering of the retained precipitated mRNA occurs after the retained precipitated mRNA has been washed using a wash buffer. As outlined above, the use of centrifuge speeds exerting reduced gravitational (g) force on the precipitated mRNA ensures that the cake of precipitated mRNA is less compact (i.e. less dense) compared to the methods of the prior art. This reduces the possibility of a residual heel forming which can cause issues when attempting to collect the retained mRNA from the filter of the filtering centrifuge. The use of filtration aid may further reduce that possibility, but its use is not necessary in order to take advantage of the improvements of the present invention. Accordingly, the methods of the present invention ensure that maximum retained mRNA can be easily removed from the filter without damaging the filter and without the requirement for complex technology, minimising the amount of residual mRNA on the filter that would reduce the overall yield of the purification method. In addition, avoiding damage to the filter avoids costly replacement and also ensures that the filter can be reused in subsequent purification processes.

In some embodiments, the recovering of the retained precipitated mRNA, optionally in combination with a filtration aid, from the filter of the filtering centrifuge occurs by dislodging the retained precipitated mRNA from the filter of the filtering centrifuge, providing a composition of precipitated mRNA, optionally in combination with a filtration aid. This composition of precipitated mRNA, optionally in combination with a filtration aid, can be either (i) stored and/or transported, or (ii) solubilised in order to provide an aqueous form of mRNA, optionally in combination with a filtration aid. In some embodiments, the recovering of the retained precipitated mRNA, optionally in combination with a filtration aid, from the filter of the filtering centrifuge comprises solubilising the precipitated mRNA retained by the filter of the filtering centrifuge, providing an aqueous form of mRNA, optionally in combination with a filtration aid. In some embodiments, the aqueous solution of mRNA, optionally in combination with a filtration aid, can be collected via centrifugation to provide purified mRNA, optionally retaining the filtration aid on the filter of the filtering centrifuge.

### Recovering a Composition of Precipitated mRNA

In some embodiments, the recovering the retained precipitated mRNA from the filter of the filtering centrifuge occurs by dislodging the retained precipitated mRNA, optionally in combination with a filtration aid, from the filter of the filtering centrifuge. As outlined above, the less dense cake of precipitated mRNA, optionally in combination with a filtration aid, is more readily dislodged from the filter of the filtering centrifuge, ensuring recovery of maximum yields of precipitated mRNA without damaging the filter.

In some embodiments, the step of recovering the retained precipitated mRNA from the filter of the filtering centrifuge is preceded by a step of drying the retained precipitated mRNA, optionally with a filtration aid. In some embodiments, the drying is via centrifugation in the filtering centrifuge. In some embodiments, the centrifugation for drying the purified mRNA composition may be at a centrifuge speed exerting a gravitational (g) force of between about 30 g to about 350 g. In some embodiments, the centrifugation for drying the purified mRNA composition may be at a centrifuge speed exerting a gravitational (g) force of between about 100 g to about 150 g.

The methods of the present invention permit the use of a blade (or plough) within the filtering centrifuge to recover maximum amounts of the retained precipitated mRNA without requiring further manual (non-automated) steps. Accordingly, in some embodiments, the recovering the retained precipitated mRNA occurs while the filtering centrifuge is in operation. In some embodiments, the recovering the retained precipitated mRNA occurs via a blade (plough) that removes the retained precipitated mRNA from the filter of the filtering centrifuge. In some embodiments, the blade removes substantially all of the retained precipitated mRNA from the filter of the filtering centrifuge. In some embodiments, the retained precipitated mRNA is collected via a sample discharge channel of the filtering centrifuge.

In some embodiments, the recovering the retained precipitated mRNA occurs while the filtering centrifuge is not in operation. In some embodiments, the retained precipitated mRNA is recovered manually from the filter of the filtering centrifuge, for example using a separate blade or plough. In some embodiments, the retained precipitated mRNA is recovered from the filter of the filtering centrifuge after the filter is removed from the filtering centrifuge. In some embodiments, the retained precipitated mRNA is recovered directly from the basket or drum of the filtering centrifuge upon opening of the centrifuge door.

In some embodiments, the recovered precipitated mRNA is in combination with a filtration aid.

In some embodiments, after recovery of the retained precipitated mRNA, the filtering centrifuge is rinsed. In some embodiments, after recovery of the retained precipitated mRNA, the filter of the filtering centrifuge is reused.

Accordingly, the methods of the present invention provide high quantities of recovered, precipitated mRNA, optionally in combination with a filtration aid. Such compositions of precipitated mRNA are easily transported and stored in large amounts of mRNA in solid form, while avoiding the more difficult transport of equivalent amounts of mRNA in much larger volumes of aqueous solution. Therefore, the methods of the present invention provide a composition of precipitated mRNA which is substantially free from contaminants (excluding the filtration aid), salts and solvents/amphiphilic polymers, which can be easily transported and stored. At an appropriate point, the mRNA in the composition can be solubilised to allow for the further methods of the invention to be used to separate the mRNA from the filtration aid, providing purified mRNA, as outlined in detail below.

In some embodiments, the recovery of the retained precipitated mRNA provides a composition of purified precipitated mRNA. In some embodiments, the composition of purified precipitated mRNA is in a form suitable for transport and long-term storage. In some embodiments, the recovery of the retained precipitated mRNA provides a composition of precipitated mRNA in combination with a filtration aid. In some embodiments, the composition of precipitated mRNA in combination with a filtration aid is in a form suitable for transport and long-term storage.

In some embodiments, the composition of precipitated mRNA comprises a precipitated mRNA collected by any method of the invention. In some embodiments, the composition of precipitated mRNA comprises a purified mRNA precipitate prepared by any method of the invention.

Accordingly, the invention provides a composition comprising mRNA, amphiphilic polymer and a filtration aid at relative concentrations of about 1:1:10 in a sterile, RNase-free container. In some embodiments, the composition comprises 10 g, 50 g, 100 g, 200 g, 300 g, 400 g, 500 g, 600 g, 700 g, 800 g, 900 g, 1 kg, 5 kg, 10 kg, 50 kg, 100 kg, one metric ton, ten metric ton or more of mRNA. In some embodiments, the amphiphilic polymer comprises PEG having a molecular weight of about 2000-10000 g/mol; 4000-8000 g/mol or about 6000 g/mol (for example PEG-6000). In other embodiments, the amphiphilic polymer comprises MTEG. In particular embodiments, the filtration aid is cellulose-based.

In some embodiments, the composition of precipitated mRNA, optionally comprising a filtration aid, is transferred to a vessel for solubilisation of the mRNA. In some embodiments, the solubilisation of the composition of precipitated mRNA provides an aqueous solution of purified mRNA. In some embodiments, the solubilisation of the composition of precipitated mRNA provides an aqueous solution of mRNA in combination with a filtration aid. In some embodiments, the mRNA in the aqueous solution is separated from the filtration aid, for example via centrifugation in a filtering centrifuge, to provide purified mRNA, by retaining the filtration aid on the filter of the filtering centrifuge.

### Recovering the Washed Retained Precipitated mRNA in Solubilised Form

As outlined above, in some embodiments, the washed retained precipitated mRNA, optionally in combination with a filtration aid, is recovered from the filter of the filtering centrifuge by solubilising the mRNA to provide an aqueous solution of mRNA, optionally in combination with a filtration aid. Accordingly, in some embodiments, the precipitated mRNA is solubilised inside the filtering centrifuge to recover the retained precipitated mRNA from the filter of the filtering centrifuge. As outlined above, the use of centrifuge speeds exerting reduced gravitational (g) force on the precipitated mRNA ensures that the cake of precipitated mRNA is less compact (i.e. less dense), rendering the retained precipitated mRNA more readily solubilised and maximising the yield of recovered mRNA.

Exemplary aqueous media for solubilising precipitated mRNA are provided below.

In some embodiments, the recovering the retained precipitated mRNA from the filter comprises the steps of (i) solubilising the retained precipitated mRNA; and (ii) collecting the solubilised mRNA.

In some embodiments, the recovery of the retained precipitated mRNA in solubilised form provides an aqueous solution of purified mRNA. In some embodiments, the methods of the invention comprise a further step of collecting purified mRNA from the aqueous solution of mRNA, for example via centrifugation in a filtering centrifuge. In some embodiments, the recovery of the retained precipitated mRNA in solubilised form provides an aqueous solution of mRNA in combination with a filtration aid. In some embodiments, the methods of the invention comprise a further step of collecting purified mRNA from the aqueous solution of mRNA in combination with a filtration aid, for example via centrifugation in a filtering centrifuge by retaining the filtration aid on the filter of the filtering centrifuge. Accordingly, in some embodiments, the step of recovering the retained precipitated mRNA in solubilised form includes a step of collecting the solubilised mRNA, for example using centrifugation in a filtering centrifuge. Exemplary methods for collecting the purified mRNA are outlined in detail below.

In some embodiments, recovery of the retained precipitated mRNA in solubilised form recovers any residual washed retained precipitated mRNA from the filter of the filtering centrifuge, thus maximising the yield of mRNA recovered in the process without requiring additional steps. In some embodiments, recovery of the retained precipitated mRNA in solubilised form allows the filter of the filtering centrifuge to be reused.

### Solubilising the Precipitated mRNA and Collecting Purified mRNA

Typically, purified mRNA may be collected by solubilizing the precipitated mRNA into an aqueous solution and collecting the solubilised purified mRNA (e.g., by elution through the filter of the filtering centrifuge while the centrifuge is in operation). As outlined above, the methods of the present invention are advantageous because they allow significantly increase (up to 100%) recovery of purified mRNA. The use of lower centrifuge speeds to reduce the gravitational force exerted on the precipitated mRNA result in a less dense cake. This precipitated mRNA within the less dense cake is more easily solubilised in an aqueous solution because the aqueous solution can more readily and extensively penetrate the cake and thus dissolve a greater percentage of the retained mRNA. Accordingly, the methods of the invention achieve improved solubilisation efficacy allowing increased yield of purified mRNA.

### Solubilising the Precipitated mRNA

In some embodiments, the solubilising the precipitated mRNA comprising dissolving the mRNA in an aqueous medium. In some embodiments, the aqueous medium comprises water. In some embodiments, the water is RNAse free water (e.g., water for injection). In some embodiments, the aqueous medium comprises a buffer. In some embodiments, the buffer is a Tris- EDTA (TE) buffer or sodium citrate buffer. In some embodiments, the aqueous medium comprises a sugar solution. In some embodiments, the sugar solution is a sucrose or trehalose solution. In some embodiments, the aqueous solution comprises a combination of water and (i) a buffer or (ii) a sugar solution.

In some embodiments, the aqueous medium is water for injection. In particular embodiments, the aqueous medium is TE buffer. In other particular embodiments, the aqueous medium is a 10% trehalose solution. In some embodiments, the aqueous medium for solubilising the precipitated mRNA is selected because it is compatible with encapsulation of the purified mRNA, for example 10 mM sodium citrate.

In some embodiments, the solubilising the precipitated mRNA occurs within the filtering centrifuge. In some embodiments, the solubilising the precipitated mRNA recovers the washed retained precipitated mRNA from the filter of the filtering centrifuge. In some embodiments, the solubilising the precipitated mRNA occurs outside the filtering centrifuge, for example the solubilisation of a composition of precipitated mRNA recovered from the filter of the filtering centrifuge. In some embodiments, the solubilising step can include a step of solubilising residual mRNA retained on the filter of the filtering centrifuge after the step of recovering a composition of precipitated mRNA from the filter of the filtering centrifuge. In this way, a maximum amount of retained mRNA can be recovered.

### Collecting the Purified mRNA

In some embodiments, the solubilised mRNA is collected from the aqueous solution to provide purified mRNA, substantially free of any contaminants, for example filtration aid. In some embodiments, the collecting of the solubilised mRNA comprises one or more steps of separating the filtration aid from the solubilised mRNA. In some embodiments, the one or more steps for separating the filtration aid from the solubilised mRNA comprise applying the solution comprising the solubilised mRNA and filtration aid to a porous substrate (e.g. filter), wherein the filtration aid is retained by the porous substrate (e.g. filter), yielding a solution of purified mRNA.

In some embodiments, the filter has a pore size appropriate for capturing impurities (including insoluble impurities with size more than the pore size, for example a filtration aid and/or a PEG or MTEG precipitate), while letting solubilised mRNA pass through. In some embodiments, the filter has a pore size appropriate for capturing a filtration aid (e.g. a cellulose-based filtration aid having a particle size of about 20 µm or more), while letting solubilised mRNA pass through. In some embodiments, the filter has a pore size appropriate for capturing a PEG or MTEG precipitate, while letting solubilised mRNA pass through. Exemplary filter pore sizes are provided above.

In some embodiments, the solution comprising the solubilised mRNA and filtration aid is applied to a porous substrate (e.g. filter) of a filtering centrifuge by centrifugation. In some embodiments, the solubilised mRNA passes through the filter while the filtration aid is retained by the filter, providing purified mRNA, substantially free of contaminants. In some embodiments, the solubilised mRNA passes through the filter and is collected via one or more of the sample discharge ports of the filtering centrifuge. In some embodiments, the filter used in the step of collecting the purified mRNA is the same filter as that used for the steps of retaining, washing and recovering the precipitated mRNA. In some embodiments, the filter used in the step of collecting the purified mRNA is a new filter compared to that used for the steps of retaining, washing and recovering the precipitated mRNA. In some embodiments, the filter is selected according to the pore size required for the relevant steps of the method of the invention, for example to have a pore size appropriate for capturing precipitated mRNA or for letting solubilised mRNA pass through. Accordingly, the methods of the present invention may require a first filter for the steps of retaining, washing and recovering the precipitated mRNA and a second filter for the step of collecting the purified mRNA.

In some embodiments, the centrifuge speed during the collection step exerts a gravitational (g) force of less than 3100 g. In some embodiments, the centrifuge speed during the collection step exerts a gravitational (g) force of between about 1000 g and about 3000 g. In some embodiments, the centrifuge speed during the collection step exerts a gravitational (g) force equivalent to that used in the steps of retaining the precipitated mRNA and/or washing the retained precipitated mRNA. In some embodiments, the filtering centrifuge is operated at the same centrifuge speed during the collection step that was used during the loading step (b) and the washing step (c) of the purification method of the invention.

In some embodiments, the solubilised mRNA is collected in a form suitable for a pharmaceutical composition, for example having clinical grade purity.

### Optional Steps in the Purification Method

The methods described herein can be readily modified by the person of ordinary skill in the art. Exemplary modifications, including additional exemplary steps, are described herein.

In some embodiments, the methods of the present invention further comprises a step of further purifying (e.g., dialyzing, diafiltering, and/or ultrafiltering) the purified mRNA solution. In some embodiments, the purified mRNA solution is dialyzed with 1 mM sodium citrate using a 100 kDa molecular weight cut-off (MWCO) membrane.

In some embodiments, the methods of the present invention may be carried out during or subsequent to synthesis. In some embodiments, a purification step as described herein may be performed after each step of mRNA synthesis, optionally along with other purification processes, such as dialysis, diafiltration, and/or ultrafiltration; e.g., using tangential flow filtration (TFF). For example, mRNA may undergo further purification (e.g., dialysis, diafiltration, and/or ultrafiltration) to remove shortmers after initial synthesis (e.g., with or without a tail) and then be subjected to precipitation and purification as described herein, then after addition of the cap and/or tail, be purified again by precipitation and purification. In embodiments, a further purification comprises use of tangential flow filtration (TFF).

In some embodiments, the methods of the present invention can include a further step of encapsulating the purified mRNA in a liposome. In some embodiments, this step may require further concentration and/or purification of the purified mRNA. In some embodiments, the further step of encapsulating the purified mRNA in a liposome can be performed immediately after the solubilisation and collection step of the methods of the present invention as the purified mRNA can be solubilised in an aqueous medium compatible with encapsulation.

### Systems and Processes for Use With the Methods of the Invention

The invention also provides a system for purifying mRNA, wherein the system comprises: a) a first vessel for receiving precipitated mRNA; b) a second vessel for receiving wash buffer; c) a third vessel for receiving the washed precipitated mRNA and/or an aqueous medium for solubilising precipitated mRNA; d) a filtering centrifuge comprising:
i) a filter, wherein the filter is arranged and dimensioned to retain precipitated mRNA and/or a filtration aid, and to let pass solubilised mRNA;
ii) a sample feed port; and
iii) a sample discharge port;
e) a fourth vessel for receiving purified mRNA, wherein said vessel is connected to the sample discharge port of the filtering centrifuge; f) a pump configured to direct flow through the system at a rate of about 5 liter/min/m² to about 25 liter/min/m² (with respect to the surface area of the filter of the filtering centrifuge); wherein the first vessel, the second vessel and the third vessel are operably linked to an input of the pump, and wherein the sample feed port of the filtering centrifuge is connected to an output of the pump; and g) one or more valves configured to preclude simultaneous flow from the first, second and third vessels.

In some embodiments, the third and fourth vessels are optional components of the system, for example those systems for recovering a composition of retained precipitated mRNA (see (24) in FIG. 3). Accordingly, the invention also provides a system for purifying mRNA, wherein the system comprises: a) a first vessel for receiving precipitated mRNA; b) a second vessel for receiving wash buffer; c) a filtering centrifuge comprising:
i) a filter, wherein the filter is arranged and dimensioned to retain precipitated mRNA and/or a filtration aid, and to let pass solubilised mRNA;
ii) a sample feed port; and
iii) a sample discharge channel;
d) a pump configured to direct flow through the system at a rate of about 5 liter/min/m² to about 25 liter/min/m² (with respect to the surface area of the filter of the filtering centrifuge); wherein the first vessel and the second vessel are operably linked to an input of the pump, and wherein the sample feed port of the filtering centrifuge is connected to an output of the pump; and e) one or more valves configured to preclude simultaneous flow from the first and second vessels.

In some embodiments, the pump is configured to direct flow through the system at a rate determined as a function of the surface area of the filter of the filtering centrifuge (m²). In some embodiments, the pump is configured to direct flow through the system at a rate of about 10 liter/min/m² to about 20 liter/min/m². In some embodiments, the pump is configured to direct flow through the system at a rate of about 5, 6, 7, 8, 9, 10, 11, 12, 13, 14, 15, 16, 17, 18, 19, 20, 21, 22, 23, 24 or 25 liter/min/m². In particular embodiments, the pump is configured to direct flow through the system at a rate of about 15 liter/min/m² or less.

In some embodiments of the system of the present invention, the system further comprises a data processing apparatus comprising means for controlling the system to carry out any of the methods of the present invention. In some embodiments, the data processing apparatus is (a) a computer program comprising instructions or (b) a computer-readable storage medium comprising instructions.

The system can be operated using the following process: A suspension comprising precipitated mRNA is provided in the first vessel. The precipitated mRNA may be prepared by precipitation step as described above. The precipitated mRNA comprises one or more protein and/or short abortive transcript contaminants from manufacturing it (e.g., by using one or more of the synthesis steps described above). For example, the mRNA may be manufactured through in vitro synthesis. Alternatively, an in vitro synthesised mRNA preparation may be subjected to a capping and/or tailing step as described above to manufacture a capped and/or tailed mRNA. In order to purify the precipitated mRNA, a wash buffer is provided in the second vessel. The content of the first vessel is transferred into a filtering centrifuge comprising a filter, as shown schematically in FIGS. 3-5. The transferring can occur at a rate of about 5 liter/min/m² to about 25 liter/min/m² (with respect to the surface area of the filter of the filtering centrifuge) while the filtering centrifuge is in operation at a first centrifuge speed such that the precipitated mRNA is retained on the filter of the filtering centrifuge. The content of the second vessel is transferred into the filtering centrifuge, thereby washing the precipitated mRNA retained on the filter of said filtering centrifuge. The transferring occurs at a rate of about 5 liter/min/m² to about 25 liter/min/m² (with respect to the surface area of the filter of the filtering centrifuge) while the filtering centrifuge remains in operation at the first centrifuge speed, thereby washing the precipitated mRNA with the wash buffer. Once the wash step is completed, the washed precipitated mRNA can be recovered from the filter of the filtering centrifuge, for example providing a composition of precipitated mRNA via the sample discharge channel of the filtering centrifuge (see (24) in FIG. 3). The transferring can be done by pumping. In some embodiments, the pumping is performed by a single pump operably linked to the first and second vessels.

In some embodiments, the pump is configured to transfer substances from the one or more vessels for providing the suspension comprising precipitated mRNA and/or wash buffer to the filtering centrifuge at a rate of about 10 liter/min/m² to about 20 liter/min/m² (with respect to the surface area of the filter of the filtering centrifuge). In some embodiments, the rate of transfer is about 5, 6, 7, 8, 9, 10, 11, 12, 13, 14, 15, 16, 17, 18, 19, 20, 21, 22, 23, 24 or 25 liter/min/m². In particular embodiments, the rate of transfer is about 15 liter/min/m² or less.

In some embodiments, the total volume of suspension and/or wash buffer is loaded into the filtering centrifuge in between about 0.5 hours to about 8 hours, for example between about 2 hours to about 6 hours. In some embodiments, the total volume is loaded into the filtering centrifuge in about less than about 8 hours, less than about 7 hours, less than about 6 hours, less than about 5 hours, less than about 4 hours, less than about 3 hours, less than about 2 hours, less than about 1 hour, or less than about 0.5 hours. In some embodiments, the time taken to load the total volume of suspension, wash buffer and/or solubilisation buffer into the filtering centrifuge may depend on the rotor size (i.e. basket diameter) of said filtering centrifuge, for example, loading a total volume of suspension of 1000 g of precipitated mRNA into a filtering centrifuge having a rotor size of about 50 cm may take about 3 hours (see Table D). In some embodiments, the total volume of wash buffer is loaded into the filtering centrifuge in between about 0.5 hours to about 4 hours, for example by using filtering centrifuges having a rotor size (i.e. basket diameter) of about 30 cm to about 170 cm. In some embodiments, the total volume of wash buffer is loaded into the filtering centrifuge in less than about 4 hours, less than about 3 hours, less than about 2 hours, less than about 1 hour, or less than about 0.5 hours. For example, the inventors have achieved impurity removal for a batch of 1000 g of mRNA using 500 litres of wash buffer in about 80 minutes (i.e. at a wash buffer loading rate of 6 L/min or 15 L/min/m²) using a filtering centrifuge having a rotor size of about 50 cm (see Table D).

In some embodiments, one or more valves control the transferring from the first vessel and the second vessel. In some embodiments, the content of the first vessel and the content of the second vessel are transferred to the filtering centrifuge via a sample feed port. In some embodiments, the filter of the filtering centrifuge is rinsed with water for injection comprising 1% 10N NaOH after the washed precipitated mRNA is covered from the filter of the filtering centrifuge.

In some embodiments, the recovering the washed precipitated mRNA from the filter comprises the steps of solubilising the retained precipitated mRNA and collecting the solubilised mRNA. In some embodiments, the precipitated mRNA includes a filtration aid. Accordingly, in some embodiments, the process further comprises: i) solubilising the washed precipitated mRNA comprising the filtration aid, for example the composition of precipitated mRNA recovered from the filtering centrifuge after the washing step, for example in a third vessel for receiving the washed precipitated mRNA and/or an aqueous medium for solubilising precipitated mRNA; ii) transferring the solubilised mRNA from step (i) into a centrifuge at a rate of about 0.1 liter/min to about 5 liter/min, wherein the filtering centrifuge comprises a filter retaining the filtration aid; and iii) collecting the solubilised purified mRNA from the filtering centrifuge by centrifugation, for example into a fourth vessel for receiving purified mRNA. The filtering centrifuge in step (ii) can either be the same filtering centrifuge that was used for washing the precipitated mRNA, or a different filtering centrifuge. In some embodiments, the solubilised mRNA is transferred to the filtering centrifuge via a sample feed port.

In some embodiments, the transferring in step (ii) is by pumping. In some embodiments, the pump is configured to transfer the solubilised mRNA to the filtering centrifuge at a rate of about 5 liter/min/m² to about 25 liter/min/m² (with respect to the surface area of the filter of the filtering centrifuge). In some embodiments, the rate of transfer is about 10 liter/min/m² to about 20 liter/min/m². In some embodiments, the rate of transfer is about 5, 6, 7, 8, 9, 10, 11, 12, 13, 14, 15, 16, 17, 18, 19, 20, 21, 22, 23, 24 or 25 liter/min/m² . In particular embodiments, the rate of transfer is about 15 liter/min/m² or less. In some embodiments, the total volume of solubilised mRNA is loaded into the filtering centrifuge in between about 1 minute to about 90 minutes. In some embodiments, the total volume is loaded into the filtering centrifuge in less than about 90 minutes, less than about 80 minutes, less than about 70 minutes, in less than about 60 minutes, less than about 50 minutes, less than about 30 minutes, less than about 20 minutes, less than about 10 minutes, less than about 5 minutes, less than about 4 minutes, less than about 3 minutes, less than about 2 minutes or less than about 1 minute.

In some embodiments, the solubilised purified mRNA collected in step (iii) is transferred to a further vessel by pumping. In some embodiments, the pumping is by a single pump operably linked to a vessel containing the solubilised purified mRNA and/or a vessel for collecting the solubilised purified mRNA. In some embodiments, the transferring of the solubilised purified mRNA is done through a sample discharge port of the filtering centrifuge. In some embodiments, the pump is configured to transfer the solubilised purified mRNA collected from the filtering centrifuge to a vessel for collecting the solubilised purified mRNA at a rate of about 5 liter/min/m² to about 25 liter/min/m² (with respect to the surface area of the filter of the filtering centrifuge), for example about 15 liter/min/m² or less. In some embodiments, the total volume of purified mRNA is recovered from the filtering centrifuge in between about 1 minute to about 90 minutes. In some embodiments, the total volume is recovered from the filtering centrifuge in less than about 90 minutes, less than about 80 minutes, less than about 70 minutes, less than about 60 minutes, less than about 50 minutes, less than about 30 minutes, less than about 20 minutes, less than about 10 minutes, less than about 5 minutes, less than about 4 minutes, less than about 3 minutes, less than about 2 minutes or less than about 1 minute.

In some embodiments, the filter used in step (ii) of the process is the same filter as that used for retaining the precipitated mRNA on the filter of the filtering centrifuge. In some embodiments, the filter can be reused for subsequent rounds of purification. Accordingly, the process of the invention is particularly suitable for providing an efficient method of efficiently achieving large scale purification of mRNA as the process of the invention does not require an exchange of filters because the filter used in step (ii) of the process (i.e. for capturing filtration aid while letting solubilised mRNA pass through) is the same filter as that used for retaining the precipitated mRNA in combination with a filtration aid on the filter of the filtering centrifuge.

In some embodiments, the process of the invention does not require an exchange of filters because the suspension of precipitated mRNA optionally does not include a filtration aid. Accordingly, the step of recovering the precipitated mRNA from the filter of the filtering centrifuge provides purified mRNA upon solubilisation of the precipitated mRNA. Therefore, the process of the present invention provides a more straightforward process of purifying mRNA. Furthermore, the process of the invention enables repeated cycles of mRNA purification without the need for replacing the filter, thus reducing the cost and burden of purifying large scales of mRNA.

Exemplary systems and processes for use with the methods of the invention are outlined in FIGS. 3-5.

### Suitable Nucleic Acids for the Methods Described Herein

### mRNA Length

According to various embodiments, the present invention is used to purify in vitro synthesized mRNA of a variety of lengths. In some embodiments, the present invention may be used to purify in vitro synthesized mRNA of or greater than about 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, or 20 kb in length. In some embodiments, the present invention may be used to purify in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length. For example, typical mRNAs may be about 1 kb to about 5 kb in length. More typically, the mRNA will have a length of about 1 kb to about 3 kb. However, in some embodiments, the mRNA in the composition of the invention is much longer (greater than about 20 kb).

### mRNA Modifications

In some embodiments, the present invention is used to purify mRNA containing one or more modifications that typically enhance stability. In some embodiments, one or more modifications are selected from modified nucleotide, modified sugar phosphate backbones, 5′ and/or 3′ untranslated region. In some embodiments, the present invention is used to purify in vitro synthesized mRNA that is unmodified. In some embodiments, the mRNA comprises no nucleotide modifications.

Typically, mRNAs are modified to enhance stability. Modifications of mRNA can include, for example, modifications of the nucleotides of the RNA. A modified mRNA according to the invention can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. Nos. 5,262,530 and 5,700,642, the disclosure of which is included here in its full scope by reference.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs that are purified using the methods described herein include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G.

While mRNA provided from in vitro transcription reactions may be desirable in some embodiments, other sources of mRNA, including wild-type mRNA produced from bacteria, fungi, plants, and/or animals may also be purified using the methods of the present invention.

In some embodiments, mRNAs for purification in the methods described herein include a 5′ and/or 3′ untranslated region. In some embodiments, a 5′ untranslated region includes one or more elements that affect an mRNA’s stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA’s stability of location in a cell, or one or more binding sites for miRNAs. In some embodiments, a 3′ untranslated region may be between 50 and 500 nucleotides in length or longer.

The present invention can be used to purify mRNAs that encode any protein.

### The Recovered mRNA

### Scale and Recovered Amounts

A particular advantage provided by the present invention is the ability to purify mRNA, in particular, mRNA synthesized in vitro, at a large or commercial scale. For example, in some embodiments in vitro synthesized mRNA is purified at a scale of or greater than about 100 milligram, 1 gram, 10 gram, 50 gram, 100 gram, 200 gram, 300 gram, 400 gram, 500 gram, 600 gram, 700 gram, 800 gram, 900 gram, 1 kg, 5 kg, 10 kg, 50 kg, 100 kg, one metric ton, ten metric ton or more per batch. In particular embodiments, in vitro synthesized mRNA is purified at a scale of or greater than about 500 g. As demonstrated in the examples, the methods of the invention are scalable to allow the purification of batches of in vitro synthesized mRNA of at least about 500 g. In particular the methods require reduced volumes of wash buffer, thus requiring less solvent for those protocols that require solvent washes, and also allow more efficient and cost effective purification of larger batches of mRNA compared to previous methods.

In some embodiments, the scale of purified mRNA depends on the size of the basket of the filtering centrifuge. For example, a filtering centrifuge having a basket diameter of 30 cm and depth of 15 cm can accommodate a maximum load of precipitated mRNA, optionally comprising a filtration aid, of about 4 kg. In some embodiments, a filtering centrifuge having a basket diameter of 50 cm and depth of 25 cm (e.g. Rousselet Robatel EHBL 503) can accommodate a maximum load of precipitated mRNA, optionally comprising a filtration aid, of about 30 kg. In some embodiments, a filtering centrifuge having a basket diameter of 63 cm and depth of 31.5 cm (e.g. Rousselet Robatel EHBL 633) can accommodate a maximum load of precipitated mRNA, optionally comprising a filtration aid, of about 50 kg. In some embodiments, a filtering centrifuge having a basket diameter of 81 cm and depth of 35 cm (e.g. Rousselet Robatel EHBL 813) can accommodate a maximum load of precipitated mRNA, optionally comprising a filtration aid, of about 120 kg. In some embodiments, a filtering centrifuge having a basket diameter of 105 cm and depth of 61 cm (e.g. Rousselet Robatel EHBL 1053) can accommodate a maximum load of precipitated mRNA, optionally comprising a filtration aid, of about 275 kg. In some embodiments, a filtering centrifuge having a basket diameter of 115 cm and depth of 61 cm (e.g. Rousselet Robatel EHBL 1153) can accommodate a maximum load of precipitated mRNA, optionally comprising a filtration aid, of about 410 kg. In some embodiments, a filtering centrifuge having a basket diameter of 132 cm and depth of 72 cm (e.g. Rousselet Robatel EHBL 1323) can accommodate a maximum load of precipitated mRNA, optionally comprising a filtration aid, of about 550 kg.

In one particular embodiment, in vitro synthesized mRNA is purified at a scale of 10 gram per batch. In one particular embodiment, in vitro synthesized mRNA is purified at a scale of 20 gram per batch. In one particular embodiment, in vitro synthesized mRNA is purified at a scale of 25 gram per batch. In one particular embodiment, in vitro synthesized mRNA is purified at a scale of 50 gram per batch. In another particular embodiment, in vitro synthesized mRNA is purified at a scale of 100 gram per batch. In yet another particular embodiment, in vitro synthesized mRNA is purified at a scale of 1 kg per batch. In yet another particular embodiment, in vitro synthesized mRNA is purified at a scale of 10 kg per batch. In yet another particular embodiment, in vitro synthesized mRNA is purified at a scale of 100 kg per batch. In yet another particular embodiment, in vitro synthesized mRNA is purified at a scale of 1,000 kg per batch. In yet another particular embodiment, in vitro synthesized mRNA is purified at a scale of 10,000 kg per batch.

In some embodiments, the mRNA is purified at a scale of or greater than 1 gram, 5 gram, 10 gram, 15 gram, 20 gram, 25 gram, 30 gram, 35 gram, 40 gram, 45 gram, 50 gram, 75 gram, 100 gram, 150 gram, 200 gram, 250 gram, 300 gram, 350 gram, 400 gram, 450 gram, 500 gram, 550 gram, 600 gram, 650 gram, 700 gram, 750 gram, 800 gram, 850 gram, 900 gram, 950 gram, 1 kg, 2.5 kg, 5 kg, 7.5 kg, 10 kg, 25 kg, 50 kg, 75 kg, 100 kg or more per batch.

In some embodiments, the solution comprising purified mRNA includes at least one gram, ten grams, one-hundred grams, one kilogram, ten kilograms, one-hundred kilograms, one metric ton, ten metric tons, or more mRNA, or any amount there between. In some embodiments, a method described herein is used to purify an amount of mRNA that is at least about 250 mg mRNA. In one embodiment, a method described herein is used to purify an amount of mRNA that is at least about 250 mg mRNA, about 500 mg mRNA, about 750 mg mRNA, about 1000 mg mRNA, about 1500 mg mRNA, about 2000 mg mRNA, or about 2500 mg mRNA. In embodiments, a method described herein is used to purify an amount of mRNA that is at least about 250 mg mRNA to about 500 g mRNA. In embodiments, a method described herein is used to purify an amount of mRNA that is at least about 500 mg mRNA to about 250 g mRNA, about 500 mg mRNA to about 100 g mRNA, about 500 mg mRNA to about 50 g mRNA, about 500 mg mRNA to about 25 g mRNA, about 500 mg mRNA to about 10 g mRNA, or about 500 mg mRNA to about 5 g mRNA. In embodiments, a method described herein is used to purify an amount of mRNA that is at least about 100 mg mRNA to about 10 g mRNA, about 100 mg mRNA to about 5 g mRNA, or about 100 mg mRNA to about 1 g mRNA.

In some embodiments, a method described herein provides a recovered amount of purified mRNA (or “yield”) that is at least about 40%, about 45%, about 50%, about 55%, about 60%, about 65%, about 70%, about 75%, about 80%, about 85%, about 90%, about 95% about 97%, about 98%, about 99%, or about 100%. Accordingly, in some embodiments, the recovered amount of purified mRNA is about 40%. In some embodiments, the recovered amount of purified mRNA is about 45%. In some embodiments, the recovered amount of purified mRNA is about 50%. In some embodiments, the recovered amount of purified mRNA is about 55%. In some embodiments, the recovered amount of purified mRNA is about 60%. In some embodiments, the recovered amount of purified mRNA is about 65%. In some embodiments, the recovered amount of purified mRNA is about 70%. In some embodiments, the recovered amount of purified mRNA is about 75%. In some embodiments, the recovered amount of purified mRNA is about 75%. In some embodiments, the recovered amount of purified mRNA is about 80%. In some embodiments, the recovered amount of purified mRNA is about 85%. In some embodiments, the recovered amount of purified mRNA is about 90%. In some embodiments, the recovered amount of purified mRNA is about 91%. In some embodiments, the recovered amount of purified mRNA is about 92%. In some embodiments, the recovered amount of purified mRNA is about 93%. In some embodiments, the recovered amount of purified mRNA is about 94%. In some embodiments, the recovered amount of purified mRNA is about 95%. In some embodiments, the recovered amount of purified mRNA is about 96%. In some embodiments, the recovered amount of purified mRNA is about 97%. In some embodiments, the recovered amount of purified mRNA is about 98%. In some embodiments, the recovered amount of purified mRNA is about 99%. In some embodiments, the recovered amount of purified mRNA is about 100%.

In some embodiments, the total purified mRNA is recovered in an amount that results in a yield of about 80% to about 100%. In some embodiments, the total purified mRNA is recovered in an amount that results in a yield of about 90% to about 99%. In some embodiments, the total purified mRNA is recovered in an amount that results in a yield of at least about 90%. In particular embodiments, the recovered amount of purified mRNA is more than about 80% or more than about 90%, for example between about 90% and 100%. In particular embodiments, the recovered amount of purified mRNA is more than about 95%.

### Characterisation of the Purified mRNA

The mRNA purification methods provided herein result in a purified mRNA composition that is substantially free of contaminants comprising short abortive RNA species, long abortive RNA species, double-stranded RNA (dsRNA), residual plasmid DNA, residual in vitro transcription enzymes, residual solvent and/or residual salt. As demonstrated in the examples and outlined above, the methods of the present invention achieve striking recovery of purified mRNA using reduced volumes of wash buffer and a quicker and more straightforward purification protocol compared to previous methods.

In some embodiments, the purified mRNA has a purity of about 60%. In some embodiments, the purified mRNA has a purity of about 65%. In some embodiments, the purified mRNA has a purity of about 70%. In some embodiments, the purified mRNA has a purity of about 75%. In some embodiments, the purified mRNA has a purity of about 80%. In some embodiments, the purified mRNA has a purity of about 85%. In some embodiments, the purified mRNA has a purity of about 90%. In some embodiments, the purified mRNA has a purity of about 91%. In some embodiments, the purified mRNA has a purity of about 92%. In some embodiments, the purified mRNA has a purity of about 93%. In some embodiments, the purified mRNA has a purity of about 94%. In some embodiments, the purified mRNA has a purity of about 95%. In some embodiments, the purified mRNA has a purity of about 96%. In some embodiments, the purified mRNA has a purity of about 97%. In some embodiments, the purified mRNA has a purity of about 98%. In some embodiments, the purified mRNA has a purity of about 99%. In some embodiments, the purified mRNA has a purity of about 100%. In particular embodiments, the purified mRNA has a purity of more than 99%, for example 99.9%.

In some embodiments, the purity of the purified mRNA is between about 60% and about 100%. In some embodiments, the purity of the purified mRNA is between about 80% and 99%. In particular embodiments, the purity of the purified mRNA is between about 90% and about 99%.

As outlined above, the methods of the present invention provide quicker and more straightforward procedures for obtaining large quantities of purified mRNA with clinical grade purity. In particular, the methods require reduced volumes of wash buffer to achieve significant purity and high yield of the purified mRNA. In some embodiments, the retained precipitated mRNA is washed to a purity of between about 50% to about 100% in between about 0.5 hours to about 4 hours. In some embodiments, the time taken to achieve impurity removal from the retained precipitated mRNA using a particular volume of wash buffer may depend on the rotor size (i.e. basket diameter) of said filtering centrifuge, and thus on the batch size of the precipitated mRNA, and the volume of wash buffer required (see Table D). In some embodiments, the retained precipitated mRNA is washed to a purity of at least 50%, at least 60%, at least 70%, at least 80%, at least 90%, at least 95% or about 100% in less than about 4 hours, less than about 3 hours, less than about 2 hours, less than about 1 hour, or less than about 0.5 hours.. In some embodiments, the retained precipitated mRNA is washed to a purity of more than about 95% (e.g. 99%) in less than about 90 minutes. For example, the inventors have achieved impurity removal for a batch of 1000 g of mRNA using 500 litres of wash buffer in about 80 minutes (i.e. at a wash buffer loading rate of 6 L/min or 15 L/min/m²) using a filtering centrifuge having a rotor size of about 50 cm (see Table D). Accordingly, the methods of the present invention are particularly suitable for scaling the purification of mRNA to accommodate large batches for commercial and therapeutic uses.

In some embodiments, the mRNA purified using the methods of the present invention is substantially free of one or more contaminants, for example one or more protein and/or short abortive transcript contaminants. In some embodiments, the one or more protein and/or short abortive transcript contaminants include enzyme reagents used in IVT mRNA synthesis. In some embodiments, the enzyme reagents include a polymerase enzyme (e.g., T7 RNA polymerase or SP6 RNA polymerase), DNAse I, pyrophosphatase and a capping enzyme. In some embodiments, the method also removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method also removes RNAse inhibitor. In some embodiments, the purified mRNA has a clinical grade purity without further purification.

In some embodiments, mRNA generated by the method disclosed herein has less than 10%, less than 9%, less than 8%, less than 7%, less than 6%, less than 5%, less than 4%, less than 3%, less than 2%, less than 1%, less than 0.5%, and/or less than 0.1% impurities other than full-length mRNA. The impurities include IVT contaminants, e.g., proteins, enzymes, DNA templates, free nucleotides, residual solvent, residual salt, double-stranded RNA (dsRNA), prematurely aborted RNA sequences (“shortmers” or “short abortive RNA species”), and/or long abortive RNA species. In some embodiments, the purified mRNA is substantially free of process enzymes.

In some embodiments, the residual plasmid DNA in the purified mRNA using the purification methods described herein is less than about 1 pg/mg, less than about 2 pg/mg, less than about 3 pg/mg, less than about 4 pg/mg, less than about 5 pg/mg, less than about 6 pg/mg, less than about 7 pg/mg, less than about 8 pg/mg, less than about 9 pg/mg, less than about 10 pg/mg, less than about 11 pg/mg, or less than about 12 pg/mg. Accordingly, the residual plasmid DNA in the purified mRNA using the purification methods described herein is less than about 1 pg/mg. In some embodiments, the residual plasmid DNA in the purified mRNA using the purification methods described herein is less than about 2 pg/mg. In some embodiments, the residual plasmid DNA in the purified mRNA using the purification methods described herein is less than about 3 pg/mg. In some embodiments, the residual plasmid DNA in the purified mRNA using the purification methods described herein is less than about 4 pg/mg. In some embodiments, the residual plasmid DNA in the purified mRNA using the purification methods described herein is less than about 5 pg/mg. In some embodiments, the residual plasmid DNA in the purified mRNA using the purification methods described herein is less than about 6 pg/mg. In some embodiments, the residual plasmid DNA in the purified mRNA using the purification methods described herein is less than about 7 pg/mg. In some embodiments, the residual plasmid DNA in the purified mRNA using the purification methods described herein is less than about 8 pg/mg. In some embodiments, the residual plasmid DNA in the purified mRNA using the purification methods described herein is less than about 9 pg/mg. In some embodiments, the residual plasmid DNA in the purified mRNA using the purification methods described herein is less than about 10 pg/mg. In some embodiments, the residual plasmid DNA in the purified mRNA using the purification methods described herein is less than about 11 pg/mg. In some embodiments, the residual plasmid DNA in the purified mRNA using the purification methods described herein is less than about 12 pg/mg.

In some embodiments, the present invention removes or eliminates a high degree of prematurely aborted RNA sequences (also known as “shortmers”). In some embodiments, a method according to the invention removes more than about 90%, 95%, 96%, 97%, 98%, 99% or substantially all prematurely aborted RNA sequences. In some embodiments, mRNA purified according to the present invention is substantially free of prematurely aborted RNA sequences. In some embodiments, mRNA purified according to the present invention contains less than about 5% (e.g., less than about 4%, 3%, 2%, or 1%) of prematurely aborted RNA sequences. In some embodiments, mRNA purified according to the present invention contains less than about 1% (e.g., less than about 0.9%, 0.8%, 0.7%, 0.6%, 0.5%, 0.4%, 0.3%, 0.2%, or 0.1%) of prematurely aborted RNA sequences. In some embodiments, mRNA purified according to the present invention contains undetectable prematurely aborted RNA sequences as determined by, e.g., high-performance liquid chromatography (HPLC) (e.g., shoulders or separate peaks), eithidium bromide, Coomassie staining, capillary electrophoresis or Glyoxal gel electrophoresis (e.g., presence of separate lower band). As used herein, the term “shortmers”, “short abortive RNA species”, “prematurely aborted RNA sequences” or “long abortive RNA species” refers to any transcripts that are less than full-length. In some embodiments, “shortmers”, “short abortive RNA species”, or “prematurely aborted RNA sequences” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail. In some embodiments, prematurely aborted RNA transcripts comprise less than 15 bases (e.g., less than 14, 13, 12, 11, 10, 9, 8, 7, 6, 5, 4, or 3 bases). In some embodiments, the prematurely aborted RNA transcripts contain about 8-15, 8-14, 8-13, 8-12, 8-11, or 8-10 bases.

In some embodiments, a method according to the present invention removes or eliminates a high degree of enzyme reagents used in in vitro synthesis including, but not limited to, T7 RNA polymerase, DNAse I, pyrophosphatase, and/or RNAse inhibitor. In some embodiments, the present invention is particularly effective to remove T7 RNA polymerase. In some embodiments, a method according to the invention removes more than about 90%, 95%, 96%, 97%, 98%, 99% or substantially all enzyme reagents used in in vitro synthesis including. In some embodiments, mRNA purified according to the present invention is substantially free of enzyme reagents used in in vitro synthesis including. In some embodiments, mRNA purified according to the present invention contains less than about 5% (e.g., less than about 4%, 3%, 2%, or 1%) of enzyme reagents used in in vitro synthesis including. In some embodiments, mRNA purified according to the present invention contains less than about 1% (e.g., less than about 0.9%, 0.8%, 0.7%, 0.6%, 0.5%, 0.4%, 0.3%, 0.2%, or 0.1%) of enzyme reagents used in in vitro synthesis including. In some embodiments, mRNA purified according to the present invention contains undetectable enzyme reagents used in in vitro synthesis including as determined by, e.g., silver stain, gel electrophoresis, high-performance liquid chromatography (HPLC), ultra-performance liquid chromatography (UPLC), and/or capillary electrophoresis, ethidium bromide and/or Coomassie staining.

In various embodiments, mRNA purified using a method described herein maintain high degree of integrity. As used herein, the term “mRNA integrity” generally refers to the quality of mRNA after purification. mRNA integrity may be determined using methods well known in the art, for example, by RNA agarose gel electrophoresis. In some embodiments, mRNA integrity may be determined by banding patterns of RNA agarose gel electrophoresis. In some embodiments, mRNA purified according to present invention shows little or no banding compared to reference band of RNA agarose gel electrophoresis. In some embodiments, mRNA purified according to the present invention has an integrity greater than about 80%, about 85% or about 90%. In some embodiments, mRNA purified according to the present invention has an integrity greater than about 95% (e.g., greater than about 96%, 97%, 98%, 99% or more). In some embodiments, mRNA purified according to the present invention has an integrity greater than 98%. In some embodiments, mRNA purified according to the present invention has an integrity greater than 99%. In some embodiments, mRNA purified according to the present invention has an integrity of approximately 100%. In some embodiments, a method described herein provides a composition having an increased activity, e.g., at least two-fold, three-fold, four-fold, five-fold, or more, of translated polypeptides relative to a composition having a lower percentage of full-length mRNA molecules. In some embodiments, percentage integrity can be assessed by determining the % area under the curve of the main product peak, relating to full length mRNA) of an HPLC chromatogram.

In some embodiments, the purified mRNA has an integrity of at least about 80%, 85%, 90%, 92%, 94%, 95%, 96%, 97%, 98%, or 99%. In some embodiments, the purified mRNA has an integrity of or greater than about 95%. In some embodiments, the purified mRNA has an integrity of or greater than about 98%. In particular embodiments, the purified mRNA has an integrity of or greater than about 99%.

In some embodiments, the methods of the present invention include a further step of characterising the purified mRNA. In some embodiments, the further step of characterising the purified mRNA comprises assessing one or more of the following characteristics of the purified mRNA: appearance, identity, quantity, concentration, presence of impurities, microbiological assessment, pH level and activity. In some embodiments, acceptable appearance includes a clear, colorless solution, essentially free of visible particulates. In some embodiments, the identity of the mRNA is assessed by sequencing methods. In some embodiments, the concentration is assessed by a suitable method, such as UV spectrophotometry. In some embodiments, a suitable concentration is between about 90% and 110% nominal (0.9-1.1 mg/mL).

In some embodiments, the further step of characterising the purified mRNA comprises assessment of mRNA integrity, assessment of residual plasmid DNA, and assessment of residual solvent. In some embodiments, the further step for assessing mRNA integrity comprises agarose gel electrophoresis. The gels are analyzed to determine whether the banding pattern and apparent nucleotide length is consistent with an analytical reference standard. In some embodiments, a positive control is used as a comparator on the silver stain from agarose gel electrophoresis to determine the % purity of the mRNA. In some embodiments, the further step comprises assessing RNA integrity include, for example, assessment of the purified mRNA using capillary gel electrophoresis (CGE). In some embodiments, acceptable purity of the purified mRNA as determined by CGE is that the purified mRNA composition has no greater than about 55% long abortive/degraded species. In some embodiments, the further step comprises assessing residual plasmid DNA by methods in the art, for example by the use of qPCR. In some embodiments, less than 10 pg/mg (e.g., less than 10 pg/mg, less than 9 pg/mg, less than 8 pg/mg, less than 7 pg/mg, less than 6 pg/mg, less than 5 pg/mg, less than 4 pg/mg, less than 3 pg/mg, less than 2 pg/mg, or less than 1 pg/mg) is an acceptable level of residual plasmid DNA. In some embodiments, acceptable residual solvent levels are not more than 10,000 ppm, 9,000 ppm, 8,000 ppm, 7,000 ppm, 6,000 ppm, 5,000 ppm, 4,000 ppm, 3,000 ppm, 2,000 ppm, 1,000 ppm. Accordingly, in some embodiments, acceptable residual solvent levels are not more than 10,000 ppm. In some embodiments, acceptable residual solvent levels are not more than 9,000 ppm. In some embodiments, acceptable residual solvent levels are not more than 8,000 ppm. In some embodiments, acceptable residual solvent levels are not more than 7,000 ppm. In some embodiments, acceptable residual solvent levels are not more than 6,000 ppm. In some embodiments, acceptable residual solvent levels are not more than 5,000 ppm. In some embodiments, acceptable residual solvent levels are not more than 4,000 ppm. In some embodiments, acceptable residual solvent levels are not more than 3,000 ppm. In some embodiments, acceptable residual solvent levels are not more than 2,000 ppm. In some embodiments, acceptable residual solvent levels are not more than 1,000 ppm.

In some embodiments, the further step comprises performing microbiological tests on the purified mRNA, which include, for example, assessment of bacterial endotoxins. In some embodiments, bacterial endotoxins are < 0.5 EU/mL, <0.4 EU/mL, <0.3 EU/mL, <0.2 EU/mL or <0.1 EU/mL. Accordingly, in some embodiments, bacterial endotoxins in the purified mRNA are < 0.5 EU/mL. In some embodiments, bacterial endotoxins in the purified mRNA are < 0.4 EU/mL. In some embodiments, bacterial endotoxins in the purified mRNA are < 0.3 EU/mL. In some embodiments, bacterial endotoxins in the purified mRNA are < 0.2 EU/mL. In some embodiments, bacterial endotoxins in the purified mRNA are < 0.2 EU/mL. In some embodiments, bacterial endotoxins in the purified mRNA are < 0.1 EU/mL. In some embodiments, the purified mRNA has not more than 1 CFU/10 mL, 1 CFU/25 mL, 1CFU/50 mL, 1CFU/75 mL, or not more than 1 CFU/100 mL. Accordingly, in some embodiments, the purified mRNA has not more than 1 CFU/10 mL. In some embodiments, the purified mRNA has not more than 1 CFU/25 mL. In some embodiments, the purified mRNA has not more than 1 CFU/50 mL. In some embodiments, the purified mRNA has not more than 1 CFR/75 mL. In some embodiments, the purified mRNA has 1 CFU/100 mL.

In some embodiments, the further step comprises assessing the pH of the purified mRNA. In some embodiments, acceptable pH of the purified mRNA is between 5 and 8. Accordingly, in some embodiments, the purified mRNA has a pH of about 5. In some embodiments, the purified mRNA has a pH of about 6. In some embodiments, the purified mRNA has a pH of about 7. In some embodiments, the purified mRNA has a pH of about 7. In some embodiments, the purified mRNA has a pH of about 8.

In some embodiments, the further step comprises assessing the translational fidelity of the purified mRNA. The translational fidelity can be assessed by various methods and include, for example, transfection and Western blot analysis. Acceptable characteristics of the purified mRNA includes banding pattern on a Western blot that migrates at a similar molecular weight as a reference standard.

In some embodiments, the further step comprises assessing the purified mRNA for conductance. In some embodiments, acceptable characteristics of the purified mRNA include a conductance of between about 50% and 150% of a reference standard.

In some embodiments, the further step comprises assessing the purified mRNA for Cap percentage and for PolyA tail length. In some embodiments, an acceptable Cap percentage includes Cap1, % Area: NLT90. In some embodiments, an acceptable PolyA tail length is about 100 -1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides). Accordingly, in some embodiments an acceptable PolyA tail length is about 100 nucleotides. In some embodiments, an acceptable PolyA tail length is about 200 nucleotides. In some embodiments, an acceptable PolyA tail length is about 250 nucleotides. In some embodiments, an acceptable PolyA tail length is about 300 nucleotides. In some embodiments, an acceptable PolyA tail length is about 350 nucleotides. In some embodiments, an acceptable PolyA tail length is about 400 nucleotides. In some embodiments, an acceptable PolyA tail length is about 450 nucleotides. In some embodiments, an acceptable PolyA tail length is about 500 nucleotides. In some embodiments, an acceptable PolyA tail length is about 550 nucleotides. In some embodiments, an acceptable PolyA tail length is about 600 nucleotides. In some embodiments, an acceptable PolyA tail length is about 650 nucleotides. In some embodiments, an acceptable PolyA tail length is about 700 nucleotides. In some embodiments, an acceptable PolyA tail length is about 750 nucleotides. In some embodiments, an acceptable PolyA tail length is about 800 nucleotides. In some embodiments, an acceptable PolyA tail length is about 850 nucleotides. In some embodiments, an acceptable PolyA tail length is about 900 nucleotides. In some embodiments, an acceptable PolyA tail length is about 950 nucleotides. In some embodiments, an acceptable PolyA tail length is about 1000 nucleotides. In some embodiments, an acceptable PolyA tail length is about 1100 nucleotides. In some embodiments, an acceptable PolyA tail length is about 1200 nucleotides. In some embodiments, an acceptable PolyA tail length is about 1300 nucleotides. In some embodiments, an acceptable PolyA tail length is about 1400 nucleotides. In some embodiments, an acceptable PolyA tail length is about 1500 nucleotides.

In some embodiments, the further step comprises assessing the purified mRNA for any residual PEG, for example using ultra performance liquid chromatography (UPLC) and/or mass spectrometry (MS) analysis. In some embodiments, the purified mRNA has less than between 10 ng PEG/mg of purified mRNA and 1000 ng PEG/mg of mRNA. Accordingly, in some embodiments, the purified mRNA has less than about 10 ng PEG/mg of purified mRNA. In some embodiments, the purified mRNA has less than about 100 ng PEG/mg of purified mRNA. In some embodiments, the purified mRNA has less than about 250 ng PEG/mg of purified mRNA. In some embodiments, the purified mRNA has less than about 500 ng PEG/mg of purified mRNA. In some embodiments, the purified mRNA has less than about 750 ng PEG/mg of purified mRNA. In some embodiments, the purified mRNA has less than about 1000 ng PEG/mg of purified mRNA.

Various methods of detecting and quantifying mRNA purity are known in the art. For example, such methods include, blotting, capillary electrophoresis, chromatography, fluorescence, gel electrophoresis, HPLC, silver stain, spectroscopy, ultraviolet (UV), or UPLC, or a combination thereof. In some embodiments, mRNA is first denatured by a Glyoxal dye before gel electrophoresis (“Glyoxal gel electrophoresis”). In some embodiments, the methods of the present invention comprise a further step of characterizing the synthesized mRNA before capping or tailing. In some embodiments, the methods of the present invention comprise a further step of characterizing the synthesized mRNA after capping and tailing.

In some embodiments, the further step comprises determining the % of protein contaminants in the purified mRNA by capillary electrophoresis. In some embodiments, the purified mRNA comprises 5% or less, 4% or less, 3% or less, 2% or less, 1 % or less or is substantially free of protein contaminants as determined by capillary electrophoresis. In some embodiments, the further step comprises determining the % of salt contaminants in the purified mRNA by high performance liquid chromatography (HPLC). In some embodiments, the purified mRNA comprises less than 5%, less than 4%, less than 3%, less than 2%, less than 1%, or is substantially free of salt contaminants determined by HPLC. In some embodiments, the further step comprises determining the % of short abortive transcript contaminants in the purified mRNA by HPLC. In some embodiments, the purified mRNA comprises 5% or less, 4% or less, 3% or less, 2% or less, 1 % or less or is substantially free of short abortive transcript contaminants determined by HPLC. In some embodiments, the further step comprises determining the % integrity of the purified mRNA by capillary electrophoresis. In some embodiments, the purified mRNA has integrity of 95% or greater, 96% or greater, 97% or greater, 98% or greater, or 99% or greater as determined by capillary electrophoresis.

In particular embodiments, the clinical grade purity is achieved without the further purification selected from high performance liquid chromatography (HPLC) purification, ligand or binding based purification, tangential flow filtration (TFF) purification, and/or ion exchange chromatography.

### Pharmaceutical Compositions and Methods of Treatment

### Pharmaceutical Compositions

The present invention provides methods for producing a composition enriched with full-length mRNA molecules which are greater than 500 nucleotides in length and encoding for a peptide or polypeptide of interest.

The invention provides a purified mRNA prepared by any method of the present invention. The invention also provides a solution comprising a purified mRNA prepared by any method of the present invention.

The invention also provides a composition produced by any method of the present invention. In some embodiments, the composition comprises a purified mRNA obtained by any method of the invention. In some embodiments, the composition of the invention is purified mRNA in aqueous form. In some embodiments, the composition of the invention is obtained by solubilising and collecting the precipitated mRNA. In some embodiments, the composition of the invention is obtained by separating the solubilised mRNA from a filtration aid (e.g. using a filtering centrifuge) and collecting the purified mRNA. In some embodiments, the precipitated mRNA is solubilised in an aqueous medium compatible with incorporation into a pharmaceutical composition.

Accordingly, in some embodiments, the composition further comprises at least one pharmaceutically acceptable excipient (e.g., a pharmaceutical composition including the purified mRNA composition of the present invention and at least one pharmaceutically-acceptable excipient).

The present invention also provides methods for producing a therapeutic composition enriched with full-length mRNA molecules encoding a peptide or polypeptide of interest for use in the delivery to or treatment of a subject, e.g., a human subject or a cell of a human subject or a cell that is treated and delivered to a human subject.

In certain embodiments the present invention provides a method for producing a therapeutic composition enriched with full-length mRNA that encodes a peptide or polypeptide for use in the delivery to or treatment of the lung of a subject or a lung cell. In certain embodiments the present invention provides a method for producing a therapeutic composition enriched with full-length mRNA that encodes for cystic fibrosis transmembrane conductance regulator (CFTR) protein. In certain embodiments the present invention provides a method for producing a therapeutic composition enriched with full-length mRNA that encodes for ATP-binding cassette sub-family A member 3 protein. In certain embodiments the present invention provides a method for producing a therapeutic composition enriched with full-length mRNA that encodes for dynein axonemal intermediate chain 1 protein. In certain embodiments the present invention provides a method for producing a therapeutic composition enriched with full-length mRNA that encodes for dynein axonemal heavy chain 5 (DNAH5) protein. In certain embodiments the present invention provides a method for producing a therapeutic composition enriched with full-length mRNA that encodes for alpha-1-antitrypsin protein. In certain embodiments the present invention provides a method for producing a therapeutic composition enriched with full-length mRNA that encodes for forkhead box P3 (FOXP3) protein. In certain embodiments the present invention provides a method for producing a therapeutic composition enriched with full-length mRNA that encodes one or more surfactant protein, e.g., one or more of surfactant A protein, surfactant B protein, surfactant C protein, and surfactant D protein.

In certain embodiments the present invention provides a method for producing a therapeutic composition enriched with full-length mRNA that encodes a peptide or polypeptide for use in the delivery to or treatment of the liver of a subject or a liver cell. Such peptides and polypeptides can include those associated with a urea cycle disorder, associated with a lysosomal storage disorder, with a glycogen storage disorder, associated with an amino acid metabolism disorder, associated with a lipid metabolism or fibrotic disorder, associated with methylmalonic acidemia, or associated with any other metabolic disorder for which delivery to or treatment of the liver or a liver cell with enriched full-length mRNA provides therapeutic benefit.

In certain embodiments the present invention provides a method for producing a therapeutic composition enriched with full-length mRNA that encodes for a protein associated with a urea cycle disorder. In certain embodiments the present invention provides a method for producing a therapeutic composition enriched with full-length mRNA that encodes for ornithine transcarbamylase (OTC) protein. In certain embodiments the present invention provides a method for producing a therapeutic composition enriched with full-length mRNA that encodes for arginosuccinate synthetase 1 protein. In certain embodiments the present invention provides a method for producing a therapeutic composition enriched with full-length mRNA that encodes for carbamoyl phosphate synthetase I protein. In certain embodiments the present invention provides a method for producing a therapeutic composition enriched with full-length mRNA that encodes for arginosuccinate lyase protein. In certain embodiments the present invention provides a method for producing a therapeutic composition enriched with full-length mRNA that encodes for arginase protein.

In certain embodiments the present invention provides a method for producing a therapeutic composition enriched with full-length mRNA that encodes for a protein associated with a lysosomal storage disorder. In certain embodiments the present invention provides a method for producing a therapeutic composition enriched with full-length mRNA that encodes for alpha galactosidase protein. In certain embodiments the present invention provides a method for producing a therapeutic composition enriched with full-length mRNA that encodes for glucocerebrosidase protein. In certain embodiments the present invention provides a method for producing a therapeutic composition enriched with full-length mRNA that encodes for iduronate-2-sulfatase protein. In certain embodiments the present invention provides a method for producing a therapeutic composition enriched with full-length mRNA that encodes for iduronidase protein. In certain embodiments the present invention provides a method for producing a therapeutic composition enriched with full-length mRNA that encodes for N-acetyl-alpha-D-glucosaminidase protein. In certain embodiments the present invention provides a method for producing a therapeutic composition enriched with full-length mRNA that encodes for heparan N-sulfatase protein. In certain embodiments the present invention provides a method for producing a therapeutic composition enriched with full-length mRNA that encodes for galactosamine-6 sulfatase protein. In certain embodiments the present invention provides a method for producing a therapeutic composition enriched with full-length mRNA that encodes for beta-galactosidase protein. In certain embodiments the present invention provides a method for producing a therapeutic composition enriched with full-length mRNA that encodes for lysosomal lipase protein. In certain embodiments the present invention provides a method for producing a therapeutic composition enriched with full-length mRNA that encodes for arylsulfatase B (N-acetylgalactosamine-4-sulfatase) protein. In certain embodiments the present invention provides a method for producing a therapeutic composition enriched with full-length mRNA that encodes for transcription factor EB (TFEB).

In certain embodiments the present invention provides a method for producing a therapeutic composition enriched with full-length mRNA that encodes for a protein associated with a glycogen storage disorder. In certain embodiments the present invention provides a method for producing a therapeutic composition enriched with full-length mRNA that encodes for acid alpha-glucosidase protein. In certain embodiments the present invention provides a method for producing a therapeutic composition enriched with full-length mRNA that encodes for glucose-6-phosphatase (G6PC) protein. In certain embodiments the present invention provides a method for producing a therapeutic composition enriched with full-length mRNA that encodes for liver glycogen phosphorylase protein. In certain embodiments the present invention provides a method for producing a therapeutic composition enriched with full-length mRNA that encodes for muscle phosphoglycerate mutase protein. In certain embodiments the present invention provides a method for producing a therapeutic composition enriched with full-length mRNA that encodes for glycogen debranching enzyme.

In certain embodiments the present invention provides a method for producing a therapeutic composition enriched with full-length mRNA that encodes for a protein associated with amino acid metabolism. In certain embodiments the present invention provides a method for producing a therapeutic composition enriched with full-length mRNA that encodes for phenylalanine hydroxylase enzyme. In certain embodiments the present invention provides a method for producing a therapeutic composition enriched with full-length mRNA that encodes for glutaryl-CoA dehydrogenase enzyme. In certain embodiments the present invention provides a method for producing a therapeutic composition enriched with full-length mRNA that encodes for propionyl-CoA caboxylase enzyme. In certain embodiments the present invention provides a method for producing a therapeutic composition enriched with full-length mRNA that encodes for oxalase alanine-glyoxylate aminotransferase enzyme.

In certain embodiments the present invention provides a method for producing a therapeutic composition enriched with full-length mRNA that encodes for a protein associated with a lipid metabolism or fibrotic disorder. In certain embodiments the present invention provides a method for producing a therapeutic composition enriched with full-length mRNA that encodes for a mTOR inhibitor. In certain embodiments the present invention provides a method for producing a therapeutic composition enriched with full-length mRNA that encodes for ATPase phospholipid transporting 8B1 (ATP8B1) protein. In certain embodiments the present invention provides a method for producing a therapeutic composition enriched with full-length mRNA that encodes for one or more NF-kappa B inhibitors, such as one or more of I-kappa B alpha, interferon-related development regulator 1 (IFRD1), and Sirtuin 1 (SIRT1). In certain embodiments the present invention provides a method for producing a therapeutic composition enriched with full-length mRNA that encodes for PPAR-gamma protein or an active variant.

In certain embodiments the present invention provides a method for producing a therapeutic composition enriched with full-length mRNA that encodes for a protein associated with methylmalonic acidemia. For example, in certain embodiments the present invention provides a method for producing a therapeutic composition enriched with full-length mRNA that encodes for methylmalonyl CoA mutase protein. In certain embodiments the present invention provides a method for producing a therapeutic composition enriched with full-length mRNA that encodes for methylmalonyl CoA epimerase protein.

In certain embodiments the present invention provides a method for producing a therapeutic composition enriched with full-length mRNA for which delivery to or treatment of the liver can provide therapeutic benefit. In certain embodiments the present invention provides a method for producing a therapeutic composition enriched with full-length mRNA that encodes for ATP7B protein, also known as Wilson disease protein. In certain embodiments the present invention provides a method for producing a therapeutic composition enriched with full-length mRNA that encodes for porphobilinogen deaminase enzyme. In certain embodiments the present invention provides a method for producing a therapeutic composition enriched with full-length mRNA that encodes for one or clotting enzymes, such as Factor VIII, Factor IX, Factor VII, and Factor X. In certain embodiments the present invention provides a method for producing a therapeutic composition enriched with full-length mRNA that encodes for human hemochromatosis (HFE) protein.

In certain embodiments the present invention provides a method for producing a therapeutic composition enriched with full-length mRNA that encodes a peptide or polypeptide for use in the delivery of or treatment with a vaccine for a subject or a cell of a subject. For example, in certain embodiments the present invention provides a method for producing a therapeutic composition enriched with full-length mRNA that encodes for an antigen from an infectious agent, such as a bacterium or a virus. In certain embodiments the present invention provides a method for producing a therapeutic composition enriched with full-length mRNA that encodes for an antigen from a Borrelia burgdorferi (the bacterium responsible for Lyme disease). In certain embodiments the present invention provides a method for producing a therapeutic composition enriched with full-length mRNA that encodes for an antigen from influenza virus. In certain embodiments the present invention provides a method for producing a therapeutic composition enriched with full-length mRNA that encodes for an antigen from respiratory syncytial virus. In certain embodiments the present invention provides a method for producing a therapeutic composition enriched with full-length mRNA that encodes for an antigen from rabies virus. In certain embodiments the present invention provides a method for producing a therapeutic composition enriched with full-length mRNA that encodes for an antigen from cytomegalovirus. In certain embodiments the present invention provides a method for producing a therapeutic composition enriched with full-length mRNA that encodes for an antigen from rotavirus. In certain embodiments the present invention provides a method for producing a therapeutic composition enriched with full-length mRNA that encodes for an antigen from a SARS-CoV-2 virus. In certain embodiments the present invention provides a method for producing a therapeutic composition enriched with full-length mRNA that encodes for an antigen from a hepatitis virus, such as hepatitis A virus, hepatitis B virus, or hepatis C virus. In certain embodiments the present invention provides a method for producing a therapeutic composition enriched with full-length mRNA that encodes for an antigen from human papillomavirus. In certain embodiments the present invention provides a method for producing a therapeutic composition enriched with full-length mRNA that encodes for an antigen from a herpes simplex virus, such as herpes simplex virus 1 or herpes simplex virus 2. In certain embodiments the present invention provides a method for producing a therapeutic composition enriched with full-length mRNA that encodes for an antigen from a human immunodeficiency virus, such as human immunodeficiency virus type 1 or human immunodeficiency virus type 2. In certain embodiments the present invention provides a method for producing a therapeutic composition enriched with full-length mRNA that encodes for an antigen from a human metapneumovirus. In certain embodiments the present invention provides a method for producing a therapeutic composition enriched with full-length mRNA that encodes for an antigen from a human parainfluenza virus, such as human parainfluenza virus type 1, human parainfluenza virus type 2, or human parainfluenza virus type 3. In certain embodiments the present invention provides a method for producing a therapeutic composition enriched with full-length mRNA that encodes for an antigen from malaria virus. In certain embodiments the present invention provides a method for producing a therapeutic composition enriched with full-length mRNA that encodes for an antigen from zika virus. In certain embodiments the present invention provides a method for producing a therapeutic composition enriched with full-length mRNA that encodes for an antigen from chikungunya virus.

In certain embodiments the present invention provides a method for producing a therapeutic composition enriched with full-length mRNA that encodes for an antigen associated with a cancer of a subject or identified from a cancer cell of a subject. In certain embodiments the present invention provides a method for producing a therapeutic composition enriched with full-length mRNA that encodes for an antigen determined from a subject’s own cancer cell, i.e., to provide a personalized cancer vaccine. In certain embodiments the present invention provides a method for producing a therapeutic composition enriched with full-length mRNA that encodes for an antigen expressed from a mutant KRAS gene.

### Medical Uses and Methods of Treatment

The invention also provides a method for treating a disease or disorder including a step of administering to a subject in need thereof a purified mRNA or pharmaceutical composition of the present invention. The invention further provides a method for treating a disease or disorder including a step of administering to a subject in need thereof a pharmaceutical composition of the present invention.

The invention also provides a purified mRNA of the present invention for use in therapy. The invention also provides a pharmaceutical composition of the present invention for use in therapy.

### EXAMPLES

### Example 1. Synthesis of mRNA

### IVT Reaction Conditions

In the following examples, unless otherwise described, mRNA was synthesized via in vitro transcription (IVT) using either T7 polymerase or SP6 polymerase. Briefly, in the SP6 polymerase IVT reaction, for each gram of mRNA transcribed, a reaction containing 20 mg of a linearized double stranded DNA plasmid with an RNA polymerase specific promoter, SP6 RNA polymerase, RNase inhibitor, pyrophosphatase, 5 mM NTPs, 10 mM DTT and a reaction buffer (10x -250 mM Tris-HCl, pH 7.5, 20 mM spirmidine, 50 mM NaCl ) was prepared with RNase free water then incubated at 37° C. for 60 min. The reaction was then quenched by the addition of DNase I and a DNase I buffer (10x- 100 mM Tris-HCl, 5 mM MgCl2 and 25 mM CaCl2, pH 7.6) to facilitate digestion of the double stranded DNA template in preparation for purification. The final reaction volume was 204 mL.

### 5′ Cap

Unless otherwise described the IVT transcribed mRNA was capped on its 5′ end either by including cap structures as part of the IVT reaction or in a subsequent enzymatic step. For capping as part of the IVT reaction, a cap analog can be incorporated as the first “base” in the nascent RNA strand. The cap analog may be Cap 0, Cap1, Cap 2, m6Am, or unnatural caps. Alternatively, uncapped and purified in vitro transcribed (IVT) mRNA can be modified enzymatically following IVT to include a cap, e.g., by the addition of a 5′ N7-methylguanylate Cap 0 structure using guanylate transferase and the addition of a methyl group at the 2′ O position of the penultimate nucleotide resulting in a Cap 1 structure using 2′ O-methyltransferase as described by Fechter, P.; Brownlee, G.G. “Recognition of mRNA cap structures by viral and cellular proteins” J. Gen. Virology 2005, 86, 1239-1249.

### 3′ Tail

Unless otherwise described, the IVT transcribed mRNA was tailed on its 3′ end either by including a tail template in the linearized plasmid, which tails the mRNA as part of the IVT reaction, or in a subsequent enzymatic step. For tailing as part of the IVT reaction, incorporation of a poly-T or similar tailing feature into the pDNA template is performed such that the polyA tail or similar appropriate tail is formed on the mRNA as part of the IVT process. Alternatively, a poly-A tail can be added to the 3′ end of the IVT-produced mRNA enzymatically following the IVT reaction, e.g., using poly-A polymerase.

### Example 2. Analysis of Purified mRNA

### RNA Integrity Analysis (Fragment Analyzer - Capillary Electrophoresis)

RNA integrity and tail length were assessed using a CE fragment analyzer and the commercially available RNA detection kit. Analysis of peak profiles for integrity and size shift for tail length were performed on raw data as well as normalized data sets.

### mRNA Cap Species Analysis (HPLC/MS)

Cap species present in the final purified mRNA product were quantified using the chromatographic method described in U.S. Pat. No. 9,970,047. This method is capable of accurately quantifying uncapped mRNA as a percent of total mRNA. This method also can quantify amounts of particular cap structures, such as CapG, Cap0 and Cap1 amounts, which can be reported as a percentage of total mRNA.

### dsRNA Detection (J2 Dot Blot)

The presence of double-stranded RNA (dsRNA) in individual mRNA samples was measured using the J2 anti-dsRNA dot blot previously describe by Kariko et al, Nucleic Acids Research, 2011. 39, No. 21. Briefly, either 200 ng of RNA or 25 ng of dsRNA control were blotted onto super charged Nytran. The blots were dried, blocked with 5% non-fat dry milk then probed with 1 µg of J2 antibody per blot. Blots were washed, probed with an HRP-conjugated donkey anti-mouse before being washed again. Blots were detected with ECL plus western blot detection reagent and images captured on film. Samples comprising purified mRNA were considered substantially free of dsRNA if the respective blot showed no visibly darker coloration as compared to a control that lacked any dsDNA.

### Example 3. Purification of mRNA via Centrifugation Using Reduced Centrifuge Speeds

This example demonstrates that purification of precipitated mRNA using a filtering centrifuge can achieve very high recovery of purified mRNA. In particular, this example surprisingly demonstrates that, when loading and washing of precipitated mRNA is performed at the same low speed, less wash buffer is required compared to methods that perform loading and washing at the same high centrifuge speed.

mRNA was synthesized using SP6 polymerase according to the IVT reaction and capping and tailing (C/T) reaction as described in Example 1 above. Different batch sizes of mRNA were used for this experiment. The largest batch size (500 grams) was achieved by pooling mRNA from multiple IVT synthesis reactions.

In this example, the mRNA was precipitated using a combination of the chaotropic salt guanidine thiocyanate (GSCN (5 M GSCN-10mM DTT buffer)) and the alcohol ethanol (EtOH) at a ratio of mRNA:GSCN:100% EtOH of 1:2.3:1.7. The precipitated mRNA suspension was mixed with a filtration aid (Solka-Floc) at a mRNA:filtration aid ratio of 1:10 and hen loaded as a suspension onto a filtering centrifuge, either H300P or EHBL503, depending on the size of the batch of mRNA through the sample feed port. The mRNA suspension was then retained on the filter of the filtering centrifuge by centrifugation and was subjected to washing with particular volumes of 80% EtOH before being the purified mRNA was eluted and quantified. The procedure conditions and % recovery are provided in Table B, below.

TABLE B

Conditions and % recovery

| Centrifuge | Scale (grams) | Filtration aid (grams) | Load Speed | | Wash Speed | | Wash Vol (Liter/gram) | Recovery (%) |
|---|---|---|---|---|---|---|---|---|
| | | | RPM | g | RPM | g | | |
| H300P | 10 | 100 | 3000 | 1735 | 3000 | 1735 | 8 | 91 |
| H300P | 50 | 500 | 3000 | 1735 | 3000 | 1735 | 3 | 80 |
| EHBL503 | 100 | 1000 | 1000 | 725 | 1000 | 725 | 2.5 | 100 |
| EHBL503 | 250 | 2500 | 1000 | 725 | 1000 | 725 | 1.6 | 100 |
| EHBL503 | 500 | 5000 | 1000 | 725 | 1000 | 725 | 1 | 98 |

The data in Table B demonstrate that the use of the same low speed at both the loading and washing steps achieves high % recovery of purified mRNA, while using low volumes of wash buffer. The volume of wash buffer provided in the table represents the total volume of wash buffer used in the purification process (i.e. for purifying the mRNA after (i) the IVT synthesis step and (ii) the 5′-capping and 3′-tailing steps). Accordingly, to purify the largest tested batch of mRNA, a wash volume of only 0.5 L/g mRNA is required to purify the mRNA after each manufacturing step using one cycle of the purification process. Compared to depth filtration, which requires a total volume of wash buffer of at least 4 L/g mRNA, the methods of the present invention require a 4-fold (i.e. 75%) reduction in volume of wash buffer. The quality of the purified mRNA is consistent even when low speed centrifugation is applied to larger amounts of precipitated mRNA, suggesting that the process can be scaled up to purify kilogram amounts of mRNA without a loss in purity.

### Example 4. Lower Speed Centrifugation Maintains Integrity and Purity of Purified mRNA Even at Larger Batch Sizes

This example demonstrates that integrity and purity of the mRNA can be maintained even when purifying a large scale batch of mRNA twice (after both IVT and capping and tailing (C/T) reactions) using lower speed centrifugation.

A 250 g batch of OTC mRNA was synthesised and purified as described in Example 2, with purification on a filtering centrifuge being performed after both the IVT and C/T reactions. For the purification process, the EHBL503 filtering centrifuge was operated according to the conditions for the 250 g batch provided in Table B. The integrity of the purified OTC mRNA was assessed using CE smear analysis, and the mRNA purity was assessed using silver stain analysis to detect residual process enzymes.

Strikingly, the integrity of the purified mRNA obtained using these lower centrifugation speeds was about 94% after the IVT step and about 91% after the capping and tailing step (with a tail length of 172 nucleotides). Furthermore, the silver stain analysis of the purified OTC mRNA showed no contaminants from either the IVT or capping and tailing (C/T) steps.

Therefore, the use of lower centrifuge speeds in the purification protocol maintains mRNA integrity and purity even with larger batch sizes. Accordingly, the process of the present invention, using lower centrifugation speeds, can be scaled to accommodate larger quantities of mRNA while maintaining purity and integrity of the purified mRNA suitable for clinical use.

### Example 5. Lower Speed Centrifugation Is Applicable for Ethanol-Free Purification Protocols

This example demonstrates that the advantages of using the same low centrifuge speed for loading and washing precipitated mRNA in a filtering centrifuge (namely good recovery of mRNA with low wash buffer usage) can be achieved in purification procedures that avoid volatile organic solvents such as ethanol.

CFTR mRNA was synthesized via IVT synthesis and 5′ caps and 3′ polyA tails were added as described in Example 1. The mRNA was precipitated using GSCN and an amphiphilic polymer. The amphiphilic polymer (either PEG or MTEG) was used instead of 100% ethanol. The volume ratio of mRNA, GSCN (5 M GSCN-10mM DTT buffer) and PEG or MTEG (100% weight/volume) in the precipitation reaction was 1:2.3:1. A cellulose filtration aid was added at a mRNA:filtration aid mass ratio of 1:10. The suspension was mixed at 60 Hz in a 60 L Lee vessel with a bottom-mounted impeller. The suspension was loaded into a filtering centrifuge (H300P) and washed with 95% PEG or MTEG at volumes and centrifuge speeds as summarized in Table C below. The final mRNA yield was quantified with a NanoDrop2000 spectrophotometer measuring absorbance at 280 nm. The % recovery of RNA is shown in Table C. Furthermore, the integrity of the purified mRNA was assessed using CE smear analysis, and the mRNA purity was assessed using silver stain analysis to detect residual process enzymes.

The use of PEG or MTEG as the precipitating polymer and wash buffer component resulted in recovery percentages of mRNA comparable to those observed with the ethanol-based purification methods in Example 3. Using a low centrifuge speed during loading and washing, the purification methods tested in this example required low volumes of wash buffer, comparable to the results observed in Example 3.

TABLE C

Efficient mRNA recovery using PEG or MTEG at lower centrifuge speeds

| Polymer | Scale (grams) | Filtration aid (grams) | Load Speed | | Wash Speed | | Wash Vol (Liter/gram) | Recovery (%) |
|---|---|---|---|---|---|---|---|---|
| | | | RPM | g | RPM | g | | |
| PEG | 10 | 100 | 1500 | 865 | 1500 | 865 | 1.4 | 80 |
| PEG | 10 | 100 | 1500 | 865 | 1500 | 865 | 1.4 | 82 |
| MTEG | 15 | 150 | 1500 | 865 | 1500 | 865 | 2 | 100 |

The volume of wash buffer provided in the table represents the total volume of wash buffer used in the purification process (i.e. for purifying the mRNA after (i) the IVT synthesis step and (ii) the 5′-capping and 3′-tailing steps). Accordingly, a volume of wash buffer of 1 L/g precipitated mRNA was used for each purification cycle. The use of reduced centrifuge speeds in the purification protocol with MTEG maintained mRNA integrity and purity. The integrity of the mRNA was about 82% and the purity of the mRNA achieved was about 99.9%.

Accordingly, the use lower centrifuge speeds in purification methods using filtering centrifuges ensure efficient purification of mRNA that has an integrity and purity suitable for clinical use, and this result is observed independent of whether a volatile organic solvent or an amphiphilic polymer is used.

### Example 6. Scaling Load and Wash Times Based on Filtering Centrifuge Size

Table D below outlines the predicted load and wash times of specific batch sizes of precipitated mRNA on particular filtering centrifuges, classified according to size (i.e. rotor size or basket diameter). The values are calculated on the basis of a constant system flow rate of about 15 L/min/m², a wash volume of about 0.5 L/g precipitated mRNA, and a 1:1 ratio of volume of precipitation buffer to mass of precipitated mRNA. The values can be adjusted to account for an alteration in parameters such as the system flow rate. For example, the flow rate may be varied between loading of the suspension containing the precipitated mRNA and washing of the retained precipitated mRNA on the filter of the filtering centrifuge.

TABLE D

Scaling load and wash times based on filtering centrifuge size

| Basket Diameter (mm) | Basket Depth (mm) | Filtering Surface Area (m²) | Batch scale (g) | Predicted load time (h) | Predicted wash time (h) |
|---|---|---|---|---|---|
| 300 | 150 | 0.14 | 100 | 0.8 | 0.4 |
| 500 | 250 | 0.40 | 1000 | 3.0 | 1.4 |
| 810 | 350 | 0.90 | 3917 | 4.8 | 2.4 |
| 1050 | 610 | 2.00 | 9167 | 5.1 | 2.5 |
| 1150 | 610 | 2.20 | 13750 | 6.9 | 3.5 |
| 1320 | 720 | 3.00 | 18333 | 6.8 | 3.4 |
| 1660 | 760 | 4.00 | 29583 | 8.2 | 4.1 |

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method for purifying messenger RNA (mRNA), the method comprising the steps of:
I. precipitating mRNA from a solution comprising one or more protein and/or short abortive transcript contaminants from manufacturing the mRNA to provide a suspension comprising precipitated mRNA;
II. loading the suspension comprising the precipitated mRNA into a filtering centrifuge comprising a filter wherein the precipitated mRNA is retained by the filter;
III. washing the retained precipitated mRNA by adding a wash buffer to the filtering centrifuge; and
IV. recovering the retained precipitated mRNA from the filter;
wherein the filtering centrifuge is operated during loading step (b) and washing step (c) at a centrifuge speed that exerts a gravitational (g) force of less than 1300 g.

2. The method of claim 1, wherein centrifuge speed exerts a gravitational (g) force of between about 300 g and about 1300 g, for example, between about 400 g and about 1100 g.

3. The method of claim 2, wherein centrifuge speed exerts a gravitational (g) force of between about 500 g and about 900 g, for example, between about 700 g and about 900 g, for example, between about 750 g and 850 g (e.g. about 800 g).

4. The method of claim 2, wherein centrifuge speed exerts a gravitational (g) force of between about 550 g and about 750 g, for example, between about 650 g and about 750 g.

5. The method of any one of the preceding claims, wherein the filtering centrifuge is operated at the same centrifuge speed during loading step (b) and washing step (c).

6. The method of any one of the preceding claims, wherein the recovering the retained precipitated mRNA from the filter comprises the steps of:
i. solubilising the retained precipitated mRNA; and
ii. collecting the solubilised mRNA.

7. The method of any one of the preceding claims, wherein precipitating the mRNA comprises adding one or more agents that promote precipitation of mRNA, for example one or more of an alcohol, an amphiphilic polymer, a buffer, a salt, and/or a surfactant.

8. The method of claim 7, wherein the one or more agents that promote precipitation of the mRNA are:
i. a salt, and
ii. an alcohol or an amphiphilic polymer.

9. The method of claim 7 or 8, wherein the alcohol is ethanol.

10. The method of any one of claims 7-9, wherein the salt is a chaotropic salt.

11. The method of claim 10, wherein the salt is at a final concentration of 2-4 M, for example of 2.5-3 M.

12. The method of claim 11, wherein the salt is at a final concentration of about 2.7 M.

13. The method of any one of claims 10-12, wherein the chaotropic salt is guanidinium thiocyanate (GSCN).

14. The method of claim 7, 8, or 10-13, wherein the amphiphilic polymer is selected from pluronics, polyvinyl pyrrolidone, polyvinyl alcohol, polyethylene glycol (PEG), triethylene glycol monomethyl ether (MTEG), or combinations thereof.

15. The method of claim 14, wherein the molecular weight of PEG is about 200 to about 40,000 g/mol.

16. The method of claim 15, wherein the molecular weight of PEG is about 200-600 g/mol, about 2000-10000 g/mol, or about 4000-8000 g/mol.

17. The method of claim 16, wherein the molecular weight of PEG is about 6000 g/mol (for example, PEG-6000).

18. The method of any one of claims 14-17, wherein the PEG is at a final concentration of about 10% to about 100% weight/volume.

19. The method of claim 18, wherein the PEG is at a final concentration of about 50% weight/volume.

20. The method of claim 19, wherein the PEG is at a final concentration of less than 25% weight/volume.

21. The method of claim 20, wherein the PEG is at a final concentration of about 5% to 20% weight/volume.

22. The method of claim 21, wherein the PEG is at a final concentration of about 10% to 15% weight/volume.

23. The method of claims 7, 8 or 10-14, wherein the amphiphilic polymer is MTEG.

24. The method of claim 23, wherein the MTEG is at a final concentration of about 10% to about 100% weight/volume concentration.

25. The method of claim 24, wherein the MTEG is at a final concentration of about 15% to about 45% weight/volume, for example of about 20% to about 40% weight/volume.

26. The method of claim 25, wherein the MTEG is at a final concentration of about 20%, about 25%, about 30%, or about 35% weight/volume.

27. The method of claim 26, wherein the MTEG is at a final concentration of about 25% weight/volume.

28. The method of any one of the preceding claims, wherein the suspension comprises precipitated mRNA, a salt and MTEG.

29. The method of any one of claims 26, wherein the salt is guanidinium thiocyanate (GSCN).

30. The method of any one of claims 1-8 and 10-29, wherein the suspension is free of alcohol, for example ethanol.

31. The method of any one of the preceding claims, wherein step (a) further comprises adding at least one filtration aid to the suspension comprising precipitated mRNA.

32. The method of claim 31, wherein the precipitated mRNA and the at least one filtration aid are at a mass ratio of about 1:2; about 1:5; about 1:10 or about 1:15.

33. The method of claim 32, wherein the precipitated mRNA and the at least one filtration aid are at a mass ratio of about 1:10.

34. The method of any one of claims 31-33, wherein the filtration aid is a dispersant.

35. The method of claim 34, wherein the dispersant is one or more of ash, clay, diatomaceous earth, glass beads, plastic beads, polymers, polymer beads (e.g., polypropylene beads, polystyrene beads), salts (e.g., cellulose salts), sand, and sugars.

36. The method of claim 35, wherein the polymer is a naturally occurring polymer, e.g. cellulose (for example, powdered cellulose fibre).

37. The method of any one of the preceding claims, wherein the suspension comprises at least 100 mg, 1 g, 10 g, 100 g, 250 g, 500 g, 1 kg, 10 kg, 100 kg, one metric ton, or ten metric tons, of mRNA or any amount there between.

38. The method of claim 37, wherein the suspension comprises greater than 1 kg of mRNA.

39. The method of any one of the preceding claims, wherein the filter comprises a porous substrate.

40. The method of claim 39, wherein the porous substrate is a filter cloth, a filter paper, a screen and a wire mesh.

41. The method of any one of the preceding claims, wherein the filter is a microfiltration membrane or ultrafiltration membrane.

42. The method of any one of the preceding claims, wherein the filter has an average pore size is about 0.5 micron or greater, about 0.75 micron or greater, about 1 micron or greater, about 2 microns or greater, about 3 microns or greater, about 4 microns or greater, or about 5 microns or greater.

43. The method of claim 42, wherein the filter has an average pore size of about 0.01 micron to about 200 microns, about 1 micron to about 2000 microns, about 0.2 microns to about 5 micron, or about one micron to about 3 microns, e.g. about 1 micron.

44. The method of any one of claims 40, 42 and 43, wherein the filter cloth is a polypropylene cloth having an average pore size of about 1 micron.

45. The method of any one of the preceding claims, wherein the volume of wash buffer for washing the retained precipitated mRNA is between about 0.5 L/g mRNA and about 8 L/g mRNA.

46. The method of claim 45, wherein the volume of wash buffer for washing the retained precipitated mRNA is less than 2 L/g mRNA.

47. The method of any one of the preceding claims, wherein the volume of wash buffer for washing the retained precipitated mRNA is between about 0.5 L/g mRNA and about 1.5 L/g mRNA.

48. The method of any one of the preceding claims, wherein the volume of wash buffer for washing the retained precipitated mRNA is about 0.5 L/g mRNA.

49. The method of any one of claims 45-48, wherein the wash buffer is loaded into the filtering centrifuge at a rate of about 5 liter/min/m² to about 25 liter/min/m² (with respect to the surface area of the filter of the filtering centrifuge), for example at a rate of about 10 liter/min/m² to about 20 liter/min/m², e.g. at a rate of about 15 liter/min/m².

50. The method of claim 49, wherein the total volume of wash buffer is loaded into the filtering centrifuge in between about 0.5 hours to about 4 hours, for example in less than about 90 minutes.

51. The method of claim 50, wherein the retained precipitated mRNA is washed to a purity of between about 50% to about 100% in between about 0.5 hours to about 4 hours.

52. The method of claim 51, wherein the retained precipitated mRNA is washed to a purity of at least 95%, for example about 99%, in less than about 90 minutes.

53. The method of any of the preceding claims, wherein the wash buffer comprises one or more of an alcohol, an amphiphilic polymer, a buffer, a salt, and/or a surfactant.

54. The method of claim 53 wherein the wash buffer comprises an alcohol or an amphiphilic polymer.

55. The method of claim 53 or 54, wherein the wash buffer comprises ethanol, optionally wherein the ethanol is at about 80% weight/volume concentration.

56. The method of claim 53 or 54, wherein the wash buffer comprises an amphiphilic polymer selected from pluronics, polyvinyl pyrrolidone, polyvinyl alcohol, polyethylene glycol (PEG), triethylene glycol monomethyl ether (MTEG), or combinations thereof.

57. The method of claim 56, wherein the amphiphilic polymer is PEG.

58. The method of claim 57, wherein the PEG is present in the wash solution at about 10% to about 100% weight/volume concentration.

59. The method of claim 58, wherein the PEG is present in the wash solution at about 50% to about 95% weight/volume concentration.

60. The method of claim 59, wherein the PEG is present in the wash solution at about 90% weight/volume concentration.

61. The method of any one of claims 57-60, wherein the molecular weight of the PEG is about 100 to about 1,000 g/mol.

62. The method of claim 61, wherein the molecular weight of PEG is about 200-600 g/mol.

63. The method of claim 62, wherein the molecular weight of PEG is about 400 g/mol (for example PEG-400).

64. The method of claim 56, wherein the amphiphilic polymer is MTEG.

65. The method of claim 64, wherein the MTEG is present in the wash solution at about 75%, about 80%, about 85%, about 90% or about 95% weight/volume concentration.

66. The method of claim 65, wherein the MTEG is present in the wash solution at about 90% weight/volume concentration or about 95% weight/volume concentration.

67. The method of claim 66, wherein the MTEG is present in the wash solution at about 95% weight/volume concentration.

68. The method of any one of claims 53, 54 and 56-67, wherein the wash buffer is free of alcohol, for example ethanol.

69. The method of any one of the proceeding claims, wherein the recovering the retained mRNA occurs while the filtering centrifuge is in operation.

70. The method of claim 69, wherein the recovering the retained mRNA occurs via a blade that removes the retained precipitated mRNA from the filter of the filtering centrifuge.

71. The method of any one of claims 1-70, wherein the recovering the retained mRNA occurs while the filtering centrifuge is not in operation.

72. The method of any one of claims 1-6, 10-29, 31-52 and 56-71, wherein the method is free of alcohol, for example ethanol.

73. The method of any one of claims 3-72, wherein the solubilising the retained mRNA comprises dissolving the mRNA in an aqueous medium.

74. The method of claim 73, wherein the aqueous medium comprises water, a buffer (e.g., Tris-EDTA (TE) buffer or sodium citrate buffer), a sugar solution (e.g., a sucrose or trehalose solution), or combinations thereof.

75. The method of claim 74, wherein the aqueous medium is water for injection.

76. The method of claim 74, wherein the aqueous medium is TE buffer.

77. The method of claim 74, wherein the aqueous medium is a 10% trehalose solution.

78. The method of any one of claims 73-77, wherein the solubilising occurs within the filtering centrifuge.

79. The method of any one of claims 73-77, wherein the solubilising occurs outside the filtering centrifuge.

80. The method of any one of claims 31-79, wherein the collecting of the solubilised mRNA comprises one or more steps of separating the filtration aid from the solubilised mRNA.

81. The method of claim 80, wherein the one or more steps for separating the filtration aid from the solubilised mRNA comprise applying the solution comprising the solubilised mRNA and filtration aid to a filter, wherein the filtration aid is retained by the filter, yielding a solution of purified mRNA.

82. The method of claim 81, wherein the solution comprising the solubilised mRNA and filtration aid is applied to a filter of a filtering centrifuge by centrifugation.

83. The method of claim 82, wherein the centrifuge speed exerts a gravitational (g) force of less than 3100 g, e.g., between about 1000 g and about 3000 g.

84. The method of any one of the preceding claims, wherein the filtering centrifuge is a continuous centrifuge and/or the filtering centrifuge is orientated vertically or horizontally or the centrifuge is an inverted horizontal centrifuge.

85. The method of any one of the preceding claims, wherein the filtering centrifuge comprises a sample feed port and/or a sample discharge port.

86. The method of any one of the preceding claims, method of any one of the preceding claims, wherein the mRNA suspension is loaded into the filtering centrifuge at a rate of about 1 liter/min to about 60 liter/min, e.g., at a rate of about 5 liter/min to about 45 liter/min.

87. The method of claim 86, wherein the total mRNA suspension is loaded into the filtering centrifuge in between about 0.5 hours to about 8 hours, for example in between about 2 hours to about 6 hours.

88. The method of any one of the preceding claims, wherein the manufacturing the mRNA comprises in vitro transcription (IVT) synthesis of the mRNA.

89. The method of claim 88, wherein the manufacturing the mRNA comprises a separate step of 3′-tailing of the mRNA.

90. The method of claim 89, wherein the separate step of 3′-tailing of the mRNA further comprising 5′ capping of the mRNA.

91. The method of claim 88, wherein IVT synthesis of the mRNA comprises 5′-capping and optionally 3′-tailing of the mRNA.

92. The method of any one of claims 88-91, wherein steps (a) through (d) are performed after IVT synthesis of the mRNA.

93. The method of claim 92, wherein the volume of wash buffer for washing the retained precipitated mRNA after IVT synthesis is less than 8 L/g mRNA, e.g., less than 6 L/g mRNA or less than 5 L/g mRNA.

94. The method of claim 93, wherein the volume of wash buffer for washing the retained precipitated mRNA after IVT synthesis is between about 0.5 L/g mRNA and about 4 L/g mRNA.

95. The method of claim 94, wherein the volume of wash buffer for washing the retained precipitated mRNA after IVT synthesis is between about 0.5 L/g mRNA and about 1.5 L/g mRNA.

96. The method of any one of claims 87-91, wherein steps (a) through (d) are performed after IVT synthesis of the mRNA and again after the separate step of 3′-tailing of the mRNA.

97. The method of claim 96, wherein the total volume of wash buffer for washing the retained precipitated mRNA after IVT synthesis and/or after the separate step of 3′-tailing of the mRNA is less than 8 L/g mRNA, e.g., less than 6 L/g mRNA or less than 5 L/g mRNA.

98. The method of claim 97, wherein the total volume of wash buffer for washing the retained precipitated mRNA after IVT synthesis and/or after the separate step of 3′-tailing of the mRNA is between about 0.5 L/g mRNA and about 4 L/g mRNA.

99. The method of claim 98, wherein the total volume of wash buffer for washing the retained precipitated mRNA after IVT synthesis and/or after the separate step of 3′-tailing of the mRNA is between about 0.5 L/g mRNA and about 1.5 L/g mRNA, for example about 1 L/g mRNA.

100. The method of any one of the preceding claims, wherein the mRNA is or greater than about 1 kb, 1.5 kb, 2 kb, 2.5 kb, 3 kb, 3.5 kb, 4 kb, 4.5 kb, 5 kb, 6 kb, 7 kb, 8 kb, 9 kb, 10 kb, 11 kb, 12 kb, 13 kb, 14 kb, 15 kb, or 20 kb in length.

101. The method of any one of the preceding claims, wherein the mRNA comprises one or more nucleotide modifications.

102. The method of claim 101, wherein the one or more nucleotide modifications comprises modified sugars, modified bases, and/or modified sugar phosphate backbones.

103. The method of any one of claims 1-100, wherein the mRNA is comprises no nucleotide modifications.

104. The method of any one of the preceding claims, wherein the recovery of purified mRNA is at least 10 g, 20 g, 50 g, 100 g, 250 g, 500 g, 1 kg, 5 kg, 10 kg, 50 kg, or 100 kg per single batch.

105. The method of any one of the preceding claims, wherein the total purified mRNA is recovered in an amount that results in a yield of at least about 80%, 85%, 90%, 95%, 96%, 97%, 98%, 99%, or about 100%.

106. The method of claim 105, wherein the total purified mRNA is recovered in an amount that results in a yield of about 80% to about 100%.

107. The method of claim 106, wherein the total purified mRNA is recovered in an amount that results in a yield of about 90% to about 99%.

108. The method of claim 107, wherein the total purified mRNA is recovered in an amount that results in a yield of at least about 90%.

109. The method of any one of the preceding claims, wherein the purity of the purified mRNA is between about 60% and about 100%.

110. The method of claim 109, wherein the purity of the purified mRNA is between about 80% and 99%.

111. The method of claim 110, wherein the purity of the purified mRNA is between about 90% and about 99%.

112. The method of any one of the preceding claims, wherein the purified mRNA has an integrity of at least about 80%, 85%, 90%, 92%, 94%, 95%, 96%, 97%, 98%, or 99%.

113. The method of claim 112, wherein the purified mRNA has an integrity of or greater than about 95%.

114. The method of claim 113, wherein the purified mRNA has an integrity of or greater than about 98%.

115. The method of claim 114, wherein the purified mRNA has an integrity of or greater than about 99%.

116. The method of any one of the preceding claims, wherein the purified mRNA has a clinical grade purity without further purification.

117. The method of claim 116, wherein the purified mRNA comprises 5% or less, 4% or less, 3% or less, 2% or less, 1 % or less or is substantially free of protein contaminants as determined by capillary electrophoresis.

118. The method of claim 116 or 117, wherein the purified mRNA comprises less than 5%, less than 4%, less than 3%, less than 2%, less than 1 %, or is substantially free of salt contaminants determined by high performance liquid chromatography (HPLC).16.

119. The method of any one of claims 116-118, wherein the purified mRNA comprises 5% or less, 4% or less, 3% or less, 2% or less, 1% or less or is substantially free of short abortive transcript contaminants determined by high performance liquid chromatography (HPLC).

120. The method of any one of the preceding claims, wherein the purified mRNA has integrity of 95% or greater, 96% or greater, 97% or greater, 98% or greater, or 99% or greater as determined by capillary electrophoresis.

121. The method of any one of claims 116-120, wherein the clinical grade purity is achieved without the further purification selected from high performance liquid chromatography (HPLC) purification, ligand or binding based purification, tangential flow filtration (TFF) purification, and/or ion exchange chromatography.

122. The method of any one of the preceding claims, wherein one or more protein and/or short abortive transcript contaminants include enzyme reagents used in IVT mRNA synthesis.

123. The method of claim 122, wherein the enzyme reagents include a polymerase enzyme (e.g., T7 RNA polymerase or SP6 RNA polymerase), DNAse I, pyrophosphatase and a capping enzyme.

124. The method of any one of the preceding claims, wherein the method also removes long abortive RNA species, double-stranded RNA (dsRNA), residual plasmid DNA residual solvent and/or residual salt.

125. The method of any one of the preceding claims, wherein the short abortive transcript contaminants comprise less than 15 bases.

126. The method of any one of preceding claims, wherein the short abortive transcript contaminants comprise about 8-12 bases.

127. The method of the preceding claims, wherein the method also removes RNAse inhibitor.

128. A purified mRNA obtained by the method of any one of claims 1-127.

129. A composition comprising the purified mRNA of claim 128.

130. The composition of claim 129, further comprising at least one pharmaceutically acceptable excipient.

131. A method for treating a disease or disorder comprising administering to a subject in need thereof the purified mRNA of claim 128 or the composition of claim 129 or 130.

132. The purified mRNA of claim 128 or the composition of claim 129 or 130 for use in therapy.

133. A process for purifying mRNA, the process comprising the steps of:
I. providing a suspension comprising precipitated mRNA in a first vessel, wherein the precipitated mRNA comprises one or more protein and/or short abortive transcript contaminants from manufacturing the mRNA;
II. providing a wash buffer in a second vessel;
III. transferring the content of the first vessel into a filtering centrifuge comprising a filter, wherein the transferring occurs at a rate of about 5 liter/min/m² to about 25 liter/min/m² (with respect to the surface area of the filter of the filtering centrifuge) while the filtering centrifuge is in operation at a first centrifuge speed such that the precipitated mRNA is retained on the filter of said filtering centrifuge;
IV. transferring the content of the second vessel into the filtering centrifuge, wherein the transferring occurs at a rate of about 5 liter/min/m² to about 25 liter/min/m² (with respect to the surface area of the filter of the filtering centrifuge)while the filtering centrifuge remains in operation at the first centrifuge speed, thereby washing the precipitated mRNA retained on the filter of said filtering centrifuge with the wash buffer; and
V. recovering the washed precipitated mRNA from the filter of said filtering centrifuge.

134. The process of claim 133, wherein the transferring in steps (lll) and (IV) is by pumping.

135. The process of claim 134, wherein the pumping in steps (III) and (IV) is by a single pump operably linked to the first and second vessels.

136. The process of claim 135, wherein one or more valves control the transferring from the first vessel and the second vessel.

137. The process of any one of claims 133-136, wherein the content of the first vessel and the content of the second vessel are transferred to the filtering centrifuge via a sample feed port.

138. The process of claim 133, wherein the filter of the filtering centrifuge is rinsed with water for injection comprising 1% 10N NaOH after step (V).

139. The process of any one of claims 133-138, wherein the suspension comprising precipitated mRNA includes a filtration aid.

140. The process of claim 139, further comprising:
i. solubilising the washed precipitated mRNA comprising the filtration aid, which was recovered in step (V);
ii. transferring the solubilised mRNA from step (i) into a or said filtering centrifuge at a rate of about 5 liter/min/m²to about 25 liter/min/m²with respect to the surface area of the filter of the filtering centrifuge (e.g. about 15 liter/min/m²), wherein the filtering centrifuge comprises a filter for retaining the filtration aid; and
iii. collecting the solubilised purified mRNA from the filtering centrifuge by centrifugation.

141. The process of claim 140, wherein the transferring is done through a sample feed port of the filtering centrifuge.

142. The process of claim 140 or 141, wherein step (iii) comprises collecting the solubilised purified mRNA via a sample discharge port of the filtering centrifuge.

143. A system for purifying mRNA, wherein the system comprises:
I. a first vessel for receiving precipitated mRNA;
II. a second vessel for receiving wash buffer;
III. a third vessel for receiving the washed precipitated mRNA and/or an aqueous medium for solubilising precipitated mRNA;
IV. a filtering centrifuge comprising:
i. a filter, wherein the filter is arranged and dimensioned to retain precipitated mRNA and/or a filtration aid, and to let pass solubilised mRNA;
ii. a sample feed port; and
iii. a sample discharge port;
V. a fourth vessel for receiving purified mRNA, wherein said vessel is connected to the sample discharge port of the filtering centrifuge;
VI. a pump configured to direct flow through the system at a rate of about 5 liter/min/m²to about 25 liter/min/m² with respect to the surface area of the filter of the filtering centrifuge (e.g. about 15 liter/min/m²); wherein the first vessel, the second vessel and the third vessel are operably linked to an input of the pump, and wherein the sample feed port of the filtering centrifuge is connected to an output of the pump; and
VII. one or more valves configured to preclude simultaneous flow from the first, second and third vessels.

144. The system according to claim 143, wherein the first centrifuge speed exerts a gravitational (g) force of less than 1300 g.

145. The system according to claim 144, wherein the system further comprises a data processing apparatus comprising means for controlling the system to carry out the method of claim 137.

146. The system according to claim 145, wherein the data processing apparatus is (a) a computer program comprising instructions or (b) a computer-readable storage medium comprising instructions.

147. A composition comprising mRNA, amphiphilic polymer and a filtration aid at relative concentrations of about 1:1:10 in a sterile, RNase-free container.

148. The composition of claim 147, wherein the composition comprises 10 g, 50 g, 100 g, 200 g, 300 g, 400 g, 500 g, 600 g, 700 g, 800 g, 900 g, 1 kg, 5 kg, 10 kg, 50 kg, 100 kg, one metric ton, ten metric ton or more of mRNA.

149. The composition of claim 147 or 148, wherein the amphiphilic polymer comprises PEG having a molecular weight of about 2000-10000 g/mol; 4000-8000 g/mol or about 6000 g/mol (for example PEG-6000).

150. The composition of claim 147 or 148, wherein the amphiphilic polymer comprises MTEG.

151. The composition of any one of claims 147-150, wherein the filtration aid is cellulose-based.
